(19) United States
(12) Patent Application Publication
(10) Pub. No.: US 2022/0306740 A1
(43) Pub. Date: Sep. 29, 2022

(54) BISPECIFIC BINDING MOLECULES

(71) Applicant: AbbVie Inc., North Chicago, IL (US)

(72) Inventors: Adam S. Chervin, Chicago, IL (US); Feng Dong, Lansdale, PA (US); Edward B. Reilly, Libertyville, IL (US); Jennifer D. Stone, Livertyville, IL (US); Michael K. White, Framingham, MA (US)

(21) Appl. No.: 17/516,350

(22) Filed: Nov. 1, 2021

Related U.S. Application Data
(63) Continuation of application No. 17/175,501, filed on Feb. 12, 2021, now abandoned.
(60) Provisional application No. 62/976,117, filed on Feb. 13, 2020.
(60) Provisional application No. 62/975,334, filed on Feb. 12, 2020.

(51) Int. Cl. C07K 16/28; C07K 14/725; C07K 16/18

(52) U.S. Cl. CPC C07K 16/2809; C07K 14/7051; C07K 16/18; C07K 2317/31; C07K 2317/55; C07K 2317/90; C07K 2319/30

(57) ABSTRACT

The present disclosure provides novel bispecific molecules that binds to human Survivin and human CD3, and methods of making and using the same.

### CROSS-REFERENCE TO RELATED APPLICATIONS

This application is a continuation of U.S. patent application Ser. No. 17/175,501, filed Feb. 12, 2021, which claims benefit of priority from U.S. Provisional Application No. 62/975,334, filed Feb. 12, 2020, and to U.S. Provisional Application No. 62/976,117, filed Feb. 13, 2020, the content of each of which is incorporated by reference herein in its entirety.

### FIELD OF THE INVENTION

The present application pertains to, among other things, novel bispecific molecules that bind to both human Survivin and human CD3, compositions comprising the bispecifics, nucleic acids encoding the bispecific polypeptides, and methods of making and using the same.

### SEQUENCE LISTING

Incorporated herein by reference in its entirety is a Sequence Listing entitled, “AVR-53501_ST25”, comprising SEQ ID NO: 1 through SEQ ID NO: 90, which includes the amino acid and polynucleotide sequences disclosed herein. The Sequence Listing has been submitted herewith in ASCII text format via EFS. The Sequence Listing was first created on Feb. 12, 2021 and is 77,218 bytes in size.

### BACKGROUND OF THE INVENTION

Cancer therapies comprise a wide range of therapeutic approaches including surgery, radiation, and chemotherapy. Many existing therapeutics suffer from disadvantages, such as a lack of selectivity of targeting cancer cells over healthy cells, and the development of resistance by the cancer to the treatment.

Recent approaches based on targeted therapeutics, which preferentially affect cancer cells over normal cells, have led to chemotherapeutic regimens with fewer side effects as compared to non-targeted therapies such as radiation treatment.

Cancer immunotherapy, including agents that empower patient T-cells to kill cancer cells, has emerged as a promising therapeutic approach. T cell receptors, unlike antibodies, have evolved to recognize intracellular proteins processed as small peptides that are complexed to major histocompatibility complex (MHC) antigens, also known as human leukocyte antigens (HLA), on the cell surface.

Soluble T cell receptors (sTCRs) represent a novel class of therapeutics with the potential to target tumor-selective antigens in both hematological and solid tumors which are not currently accessible using traditional antibody-based therapeutics. However, several challenges have hindered the development of therapeutic sTCRs, including difficulty in expressing soluble, stable, and high affinity TCRs. Survivin is an attractive intracellular target overexpressed in multiple solid and hematological cancers, potentially accessible by sTCRs. Therefore, there is a need to develop a new sTCR based immunotherapeutic approach for targeting Survivin.

### BRIEF SUMMARY OF THE INVENTION

Described herein are bispecific molecules that bind to human Survivin and human CD3.

In one aspect, the present invention provides a bispecific molecule that binds to human Survivin and human CD3 comprising:
a) a single-chain T cell receptor (sTCR) comprising:
   (1) a variable beta region (V_{β}) comprising SEQ ID NO: 20 (CDR1), SEQ ID NO: 24 (CDR2), and SEQ ID NO: 28 (CDR3), and
   (2) a variable alpha region (V_{α}) comprising SEQ ID NO: 19 (CDR1), SEQ ID NO: 23 (CDR2), and SEQ ID NO: 27 (CDR3),
      wherein the V_{β} and V_{α} regions of the sTCR are connected via a first peptide linker (L1);
b) a Fab comprising:
   (1) a heavy chain region comprising a heavy chain variable (V_{H}) comprising SEQ ID NO: 21 (CDR1), SEQ ID NO: 25 (CDR2), and SEQ ID NO: 29 (CDR3); and a heavy chain constant domain 1 (CH1) comprising the amino acid sequence of SEQ ID NO: 18, and
   (2) a light chain region comprising a light chain variable (V_{L}) comprising SEQ ID NO: 22 (CDR1), SEQ ID NO: 26 (CDR2), and SEQ ID NO: 30 (CDR3); and a kappa constant light chain (C_{κ}) comprising the amino acid sequence of SEQ ID NO: 17,
      wherein the V_{α} of the sTCR and the V_{H} of the Fab are connected via a second peptide linker (L2); and
c) a Fc region comprising:
   (1) a first constant region comprising a first constant domain 2 (CH2) and a first constant domain 3 (CH3), wherein the first constant region (CH2CH3) comprises the amino acid sequence of SEQ ID NO: 13, and
   (2) a second constant region comprising a second constant domain 2 (CH2′) and a second constant domain 3 (CH3′), wherein the second constant region (CH2′CH3′) comprises the amino acid sequence of SEQ ID NO: 16,

wherein the bispecific molecule is in the form of

V_{β}-L1-V_{α}-L2-V_{H}-CH1CH2CH3V_{L}-CκCH2′CH3′.

In one embodiment, the present invention provides a bispecific molecule that binds to human Survivin and human CD3 comprising:
a) a single-chain T cell receptor (sTCR) comprising:
   (1) a variable beta region (V_{β}) comprising the amino acid sequence of SEQ ID NO: 2, and
   (2) a variable alpha region (V_{α}) comprising the amino acid sequence of SEQ ID NO: 6,
      wherein the V_{β} and V_{α} regions of the sTCR are connected via a first peptide linker (L1);
b) a Fab comprising:
   (1) a heavy chain region comprising a heavy chain variable (V_{H}) comprising the amino acid sequence of SEQ ID NO: 9; and a heavy chain constant domain 1 (CH1) comprising the amino acid sequence of SEQ ID NO: 18, and
   (2) a light chain region comprising a light chain variable (V_{L}) comprising the amino acid sequence of SEQ ID NO: 11; and a kappa constant light chain (C_{κ}) comprising the amino acid sequence of SEQ ID NO: 17,
      wherein the V_{α} of the sTCR and the V_{H} of the Fab are connected via a second peptide linker (L2); and
c) a Fc region comprising:
   (1) a first constant region comprising a first constant domain 2 (CH2) and a first constant domain 3 (CH3), wherein the first constant region (CH2CH3) comprises the amino acid sequence of SEQ ID NO: 13, and
   (2) a second constant region comprising a second constant domain 2 (CH2′) and a second constant domain 3 (CH3′), wherein the second constant region (CH2′CH3′) comprises the amino acid sequence of SEQ ID NO: 16,

wherein the bispecific molecule is in the form of

V_{β}-L1-V_{α}-L2-V_{H}-CH1CH2CH3V_{L}-CκCH2′CH3′.

In another aspect, the present invention provides a bispecific molecule that binds to human Survivin and human CD3 comprising:
a) a first heavy chain region comprising
   1) a single-chain T cell receptor (sTCR) which binds to a complex of the peptide Survivin, wherein the complex comprises the amino acid sequence of SEQ ID NO: 40 and the HLA-A2 molecule, wherein the sTCR comprises:
      i. a variable beta region (V_{β}) comprising SEQ ID NO: 20 (CDR1), SEQ ID NO: 24 (CDR2), and SEQ ID NO: 28 (CDR3); and
      ii. a variable alpha region (V_{α}) comprising SEQ ID NO: 19 (CDR1), SEQ ID NO: 23 (CDR2), and SEQ ID NO: 27 (CDR3); wherein the V_{β} and the V_{α} are connected via a first peptide linker (L1);
   2) a heavy chain variable (V_{H}) comprising SEQ ID NO: 21 (CDR1), SEQ ID NO: 25 (CDR2), and SEQ ID NO: 29 (CDR3); and
   3) a first heavy chain constant region (CH1CH2CH3) comprising an amino acid sequence selected from a group consisting of SEQ ID NO: 37, SEQ ID NO: 38 and SEQ ID NO: 39;
      wherein the V_{α} of the sTCR and the V_{H} are connected via a second peptide linker (L2), and
      wherein the first heavy chain region is in the form of V_{β}-L1-V_{α}-L2-V_{H}-CH1CH2CH3;
b) a second heavy chain region (CH2′CH3′) comprising the amino acid sequence of SEQ ID NO: 16; and
c) a light chain comprising:
   1) a light chain variable (V_{L}) comprising SEQ ID NO: 22 (CDR1), SEQ ID NO: 26 (CDR2), and SEQ ID NO: 30 (CDR3); and
   2) a kappa constant light chain (C_{κ}) having the amino acid sequence of SEQ ID NO: 17;

wherein the CH2CH3 of the first heavy chain and CH2′CH3′ of the second heavy chain form a dimeric Fc region, to form a bispecific molecule with the following form:

V_{β}-L1-V_{α}-L2-V_{H}-CH1CH2CH3V_{L}-CκCH2′CH3′.

In one embodiment, the first heavy chain constant region (CH1CH2CH3) comprises the amino acid sequence of SEQ ID NO: 37.

In another aspect, the present invention provides a bispecific molecule which binds to both human CD3 and human Survivin comprising:
a) a first heavy chain region comprising
   1) a single-chain T cell receptor (sTCR) which binds to a complex of the peptide Survivin, wherein the complex comprises the amino acid sequence of SEQ ID NO: 40 and the HLA-A2 molecule, wherein the sTCR comprises:
      i. a variable beta region (V_{β}) comprising the amino acid sequence of SEQ ID NO: 2; and
      ii. a variable alpha region (V_{α}) comprising the amino acid sequence of SEQ ID NO: 6;
         wherein the V_{β} and the V_{α} are connected via a first peptide linker (L1);
   2) a heavy chain variable (V_{H}) comprising the amino acid sequence of SEQ ID NO: 9; and
   3) a first heavy chain constant region (CH1CH2CH3) comprising the amino acid sequence of SEQ ID NO: 37;
      wherein the V_{α} of the sTCR and the V_{H} are connected via a second peptide linker (L2), and
      wherein the first heavy chain region is in the form of V_{β}-L1-V_{α}-L2-V_{H}-CH1CH2CH3;
   b) a second heavy chain region (CH2′CH3′) comprising the amino acid sequence of SEQ ID NO: 16; and
   c) a light chain comprising:
      1) a light chain variable (V_{L}) comprising the amino acid sequence of SEQ ID NO: 11; and
      2) a kappa constant light chain (C_{κ}) comprising the amino acid sequence of SEQ ID NO: 17;

wherein the CH2CH3 of the first heavy chain and CH2′CH3′ of the second heavy chain form a dimeric Fc region, to form a bispecific molecule with the following form:

V_{β}-L1-V_{α}-L2-V_{H}-CH1CH2CH3V_{L}-CκCH2′CH3′.

In some embodiments, the light chain V_{L}-C_{κ} is covalently bound by a disulfide bridge to the heavy chain region V_{H}-CH1.

In another aspect, the present invention provides a bispecific molecule which binds to human Survivin and human CD3 consisting of:
(1) a first heavy chain comprising the amino acid sequence of SEQ ID NO: 36;
(2) a second heavy chain comprising the amino acid sequence of SEQ ID NO: 16; and
(3) a light chain comprising the amino acid sequence of SEQ ID NO: 76.

In another aspect, the present invention provides a bispecific molecule which binds to human Survivin and human CD3 comprising:
(1) a first heavy chain comprising the amino acid sequence of SEQ ID NO: 36;
(2) a second heavy chain comprising the amino acid sequence of SEQ ID NO: 16; and
(3) a light chain comprising the amino acid sequence of SEQ ID NO: 76.

In certain embodiments, the light chain is linked to the first heavy chain by a disulfide bridge between the cysteine in position 489 of the first heavy chain (e.g., the cysteine in position 489 of SEQ ID NO: 36) and the cysteine in position 213 of the light chain (e.g., the cysteine in position 213 of SEQ ID NO: 76). In embodiments, the first heavy chain and the second heavy chain are connected by two disulfide bridges, where the two disulfide bridges are between the cysteine in position 495 of the first heavy chain (e.g., the cysteine in position 495 of SEQ ID NO: 36) and the cysteine in position 6 of the second heavy chain (e.g., the cysteine in position 6 of SEQ ID NO: 16), and between the cysteine in position 498 of the first heavy chain (e.g., the cysteine in position 498 of SEQ ID NO: 36) and the cysteine in position 9 of the second heavy chain (e.g., the cysteine in position 9 of SEQ ID NO: 16).

In some embodiments, the bispecific molecules provided herein lack the C-terminal lysine in the first heavy chain and/or the second heavy chain, resulting in a C-terminal glycine residue.

### BRIEF DESCRIPTION OF THE FIGURES

FIGS. 1A-1E show the illustrations for the bispecific binding proteins of the present invention. FIG. 1A depicts V_{α}V_{β}-FTab; FIG. 1B depicts V_{β}V_{α}-FTab-hb-1, V_{β}V_{α}-FTab-hb-2, V_{β}V_{α}-FTab-hb-3, V_{β}V_{α}-FTab-hb-4, and V_{β}V_{α}-FTab-hb-5; FIG. 1C depicts V_{β}V_{α}-FTab-KiH and V_{β}V_{α}-FTab-KiH-2; FIG. 1D depicts V_{β}V_{α}-FTab-1, V_{β}V_{α}-FTab-2, and V_{β}V_{α}-FTab-3; and FIG. 1E depicts V_{α}V_{β}-FTab-hb-1.

FIG. 2 shows Survivin TCR/CD3 bispecific molecule V_{β}V_{α}-FTab-KiH induced potent killing of OCI-AML2 across 4 healthy CD3+ T cell donors, as described in Example 4.

FIG. 3 shows Survivin TCR/CD3 bispecific molecule V_{β}V_{α}-FTab-KiH induced potent killing of OCI-AML3 across 4 healthy CD3+ T cell donors, as described in Example 4.

FIG. 4 shows Survivin TCR/CD3 bispecific molecule V_{β}V_{α}-FTab-KiH did not induce killing of OCI-Ly19, as described in Example 4.

FIG. 5 shows Survivin TCR/CD3 bispecific molecule V_{β}V_{α}-FTab-KiH induced potent activation of CD3+ T cells across 4 healthy CD3+ T cell donors, against OCI-AML2, as described in Example 6.

FIG. 6 shows Survivin TCR/CD3 bispecific molecule V_{β}V_{α}-FTab-KiH induced potent activation of CD3+ T cells across 4 healthy CD3+ T cell donors, against OCI-AML3, as described in Example 6.

FIG. 7 shows Survivin TCR/CD3 bispecific molecule V_{β}V_{α}-FTab-KiH induced minimal activation of CD3+ T cells across 4 healthy CD3+ T cell donors, against OCI-Ly19, as described in Example 6.

FIG. 8 shows Survivin TCR/CD3 bispecific half-life in monkey serum, as described in Example 7.

FIG. 9 shows at molar equivalent doses, the bispecific molecule with KiH (i.e., V_{β}V_{α}-FTab-KiH′ which was also designated as V_{β}V_{α}-FTab-KiH-2) exhibited greater in vivo anti-tumor efficacy than the bispecific molecule that does not contain KiH (i.e., V_{β}V_{α}-FTab-hb-5), as described in Example 3.

FIG. 10 shows the schematic representation of Survivin TCR/CD3 bispecific molecule V_{β}V_{α}-FTab-KiH.

FIG. 11 shows V_{β}V_{α}-FTab-KiH TCR specificity screen.

FIG. 12 shows T cell proliferation induced by V_{β}V_{α}-FTab-KiH at varying effector to target ratios.

### DETAILED DESCRIPTION OF THE INVENTION

Described herein are novel bispecific molecules comprising an anti-CD3 binding domain and a soluble single chain T cell receptor targeting human Survivin. These bispecific molecules exhibit several unexpected properties, including, for example, unexpectedly long half-life, remarkable binding specificity directed towards a Survivin-derived peptide complexed to HLA-A2, and potent induction of T cell activation and proliferation.

### Abbreviations

The bispecific binding molecules and polynucleotides described herein are, in many embodiments, described by way of their respective polypeptide or polynucleotide sequences. Unless indicated otherwise, polypeptide sequences are provided in N-terminus to C-terminus orientation, and polynucleotide sequences in 5′→3′ orientation. For polypeptide sequences, the conventional three or one-letter abbreviations for the genetically encoded amino acids are used.

| Abbreviation | Term |
|---|---|
| MACS | magnetic-activated cell sorting |
| PBS | phosphate-buffered saline |
| BSA | bovine serum albumin |
| EDTA | ethylenediaminetetraacetic acid |
| DMSO | dimethyl sulfoxide |
| FACS | fluorescence-activated cell sorting |
| Tris | tris(hydroxymethyl)aminomethane |
| SEC | size-exclusion chromatography |
| SDS-PAGE | sodium dodecyl sulfate-polyacrylamide gel |
| | electrophoresis |
| PBMC | peripheral blood mononuclear cells |
| FBS | fetal bovine serum |
| MABEL | minimum anticipated biological effect level |
| PBMC | peripheral blood mononuclear cell |
| MeCN | Acetonitrile |
| TFA | trifluoroacetic acid |
| NMR | nuclear magnetic resonance |
| DMSO | dimethyl sulfoxide |
| LC/MS or LCMS | liquid chromatography- mass spectrometry |
| MeOH | Methanol |
| tBME | tert-butyl methyl ether |
| min | Minute |
| mL | Milliliter |
| μL | Microliter |
| g | Gram |
| mg | Milligram |
| mmol | Millimoles |
| HPLC | high pressure liquid chromatography |
| ppm | parts per million |
| pm | Micrometer |

### Embodiments

Described herein are bispecific molecules that comprise a CD3 binding part that binds to human CD3 and a Survivin binding part that binds to human Survivin.

The term “human CD3” as used herein relates to human cluster of differentiation 3 protein (CD3) described under UniProt P07766 (CD3E-HUMAN).

The term “human Survivin” or “Survivin” as used herein relates to an inhibitor of apoptosis protein (IAP) described under UniProt O15392 (BIRC5_Human) which is a tumor-associated antigen that is expressed in human cancer cells.

“Binding to CD3 or human Survivin” refers to a molecule that is capable of binding CD3 or human Survivin with sufficient affinity such that the molecule is useful as a therapeutic agent in targeting CD3 or human Survivin.

Survivin Binding Part

In one embodiment, Survivin binding part of the bispecific molecules of the present invention refers to a single-chain soluble T cell receptor (sTCR).

The term “T cell receptors (TCRs)” as used herein are antigen-specific molecules that are responsible for recognizing antigenic peptides presented in the context of a product of the major histocompatibility complex (MHC) on the surface of antigen presenting cells (APCs) or any nucleated cell (e.g., all human cells in the body, except red blood cells).

In one embodiment, the sTCR of the present invention is a modified TCR comprising a variable alpha region (V_{α}) and a variable beta region (V_{β}) derived from a wild type T cell receptor, wherein the V_{α}, the V_{β}, or both, comprise at least one mutation in one or more complementarity determining regions (CDRs) relative to the wild type T cell receptor, wherein the modified T cell receptor binds to a complex of the peptide (i.e., the Survivin peptide LTLGEFLKL (SEQ ID NO: 40)) and a MHC product known as HLA-A2 molecule.

In one embodiment, the sTCR of the present invention comprises a V_{β} and a V_{α}, wherein the sTCR binds to a complex of the peptide comprising the amino acid sequence of SEQ ID NO: 40 and the HLA-A2 molecule.

In one embodiment, the sTCR of the present invention comprises a V_{β} and a V_{α}, wherein the sTCR binds to a peptide (SEQ ID NO: 40) derived from human Survivin in complex with HLA-A2.

In embodiments, the compounds of the present disclosure bind to survivin peptide/MHC with a K_{D} of 1×10⁻⁷M or less, such as between about 1×10⁻⁷M and about 1×10⁻¹⁰ M, or between about 1×10⁻⁸M and about 1×10⁼¹⁰ M. In embodiments, the compounds of the present disclosure bind to survivin peptide/MHC complex with a K_{D} of less than about 3×10⁻⁹M, or less than about 2.5×10⁻⁹M, or less than about 2.0×10⁻⁹M, or less than about 1.5×10⁻⁹M.

In one embodiment, the V_{α} of the sTCR of the present invention comprises SEQ ID NO: 19 (CDR1), SEQ ID NO: 23 (CDR2), and SEQ ID NO: 27 (CDR3).

In one embodiment, the V_{α} of the sTCR of the present invention comprises an amino acid sequence selected from the group consisting of SEQ ID NO: 6, SEQ ID NO: 7 and SEQ ID NO: 8.

In one preferred embodiment, the V_{α} of the sTCR of the present invention comprises the amino acid sequence of SEQ ID NO: 6.

In one embodiment, the V_{β} of the sTCR of the present invention comprises SEQ ID NO: 20 (CDR1), SEQ ID NO: 24 (CDR2) and SEQ ID NO: 28 (CDR3) or SEQ ID NO: 31 (CDR3).

In one preferred embodiment, the V_{β} of the sTCR of the present invention comprises SEQ ID NO: 20 (CDR1), SEQ ID NO: 24 (CDR2) and SEQ ID NO: 28 (CDR3).

In one embodiment, the V_{β} of the sTCR of the present invention comprises an amino acid sequence selected from the group consisting of SEQ ID NO: 2, SEQ ID NO: 3, SEQ ID NO: 4 and SEQ ID NO: 5.

In one embodiment, the V_{β} of the sTCR of the present invention comprises the amino acid sequence of SEQ ID NO: 2.

In one embodiment, the sTCR variable beta region (V_{β}) and the sTCR variable alpha region (V_{α}) are connected via a first peptide linker (L1). The linker may be selected to increase expression, solubility, stability (for example, as measured by lower aggregation levels, lower rate of aggregation, higher melting temperature, and/or longer plasma half-life), and/or titer of a bispecific molecule of the present invention.

In one embodiment, the sTCR variable beta region (V_{β}) and the sTCR variable alpha region (V_{α}) are connected via a first peptide linker (L1) comprising the amino acid sequence of SEQ ID NO: 1.

In certain embodiments, the sTCR variable beta region (V_{β}) is connected to the sTCR variable alpha region (V_{α}) via a disulfide bridge. In embodiments, the disulfide bridge connecting the V_{α} and V_{β} regions is between cysteine 43 of the V_{α} region and cysteine 235 of the V_{β} region. In embodiments, the disulfide bridge connecting the V_{α} and V_{β} regions is between cysteine 43 and cysteine 235 of SEQ ID NO: 36 or SEQ ID NO: 88. In embodiments, the disulfide bridge connecting the V_{α} and V_{β} regions is between cysteine 43 of SEQ ID NO: 5 or SEQ ID NO: 2, and cysteine 100 of SEQ ID NO: 6.

In one embodiment, the single-chain soluble T cell receptor (sTCR) of the present invention, which binds to a complex of the peptide Survivin, wherein the complex comprises the amino acid sequence of SEQ ID NO: 40 and the HLA-A2 molecule, wherein the sTCR comprises:
(1) a sTCR variable beta region (V_{β}) comprising SEQ ID NO: 20 (CDR1), SEQ ID NO: 24 (CDR2), and SEQ ID NO: 28 (CDR3) or SEQ ID NO: 31 (CDR3), and
(2) a sTCR variable alpha region (V_{α}) comprising SEQ ID NO: 19 (CDR1), SEQ ID NO: 23 (CDR2), and SEQ ID NO: 27 (CDR3), wherein the V_{β} and V_{α} regions of the sTCR are connected via a first peptide linker (L1).

In one embodiment, the single-chain soluble T cell receptor (sTCR) of the present invention, which binds to a complex of the peptide Survivin, wherein the complex comprises the amino acid sequence of SEQ ID NO: 40 and the HLA-A2 molecule, wherein the sTCR comprises:
(1) a sTCR variable beta region (V_{β}) comprising SEQ ID NO: 20 (CDR1), SEQ ID NO: 24 (CDR2), and SEQ ID NO: 28 (CDR3), and
(2) a sTCR variable alpha region (V_{α}) comprising SEQ ID NO: 19 (CDR1), SEQ ID NO: 23 (CDR2), and SEQ ID NO: 27 (CDR3),

wherein the V_{β} and V_{α} regions of the sTCR are connected via a first peptide linker (L1).

In one embodiment, the single-chain soluble T cell receptor (sTCR) of the present invention, which binds to a complex of the peptide Survivin, wherein the complex comprises the amino acid sequence of SEQ ID NO: 40 and the HLA-A2 molecule, wherein the sTCR comprises:
(1) a sTCR variable beta region (V_{β}) comprising the amino acid sequence selected from a group consisting of SEQ ID NO: 2, SEQ ID NO: 3, SEQ ID NO: 4 and SEQ ID NO: 5, and
(2) a sTCR variable alpha region (V_{α}) comprising the amino acid sequence selected from a group consisting of SEQ ID NO: 6, SEQ ID NO: 7 and SEQ ID NO: 8,

wherein the V_{β} and V_{α} regions of the sTCR are connected via a first peptide linker (L1).

In one embodiment, the single-chain soluble T cell receptor (sTCR) of the present invention, which binds to a complex of the peptide Survivin, wherein the complex comprises the amino acid sequence of SEQ ID NO: 40 and the HLA-A2 molecule, wherein the sTCR comprises:
(1) a sTCR variable beta region (V_{β}) comprising the amino acid sequence selected from a group consisting of SEQ ID NO: 2, and
(2) a sTCR variable alpha region (V_{α}) comprising the amino acid sequence selected from a group consisting of SEQ ID NO: 6,

wherein the V_{β} and V_{α} regions of the sTCR are connected via a first peptide linker (L1).

In one embodiment, the first peptide linker (L1) of the present invention comprises the amino acid sequence of SEQ ID NO: 1.

CD3 Binding Part

In one embodiment, CD3 binding part of the bispecific molecules of the present invention is a combination of an antibody heavy chain comprising a heavy chain variable domain (V_{H}) and a constant heavy chain domain 1 (CH1) and an antibody light chain comprising a light chain variable domain (V_{L}) and a kappa (κ) light chain (constant domain Cκ), and preferably the V_{H}, CH1, V_{L} and Cκ as enclosed in an antigen binding fragment (Fab) that binds to human CD3 (anti-CD3-Fab), wherein the light chain (V_{L}-Cκ) is covalently bound by a disulfide bridge to the heavy chain (V_{H}—CH1). In some embodiments, the Cκ is replaced with a lambda light constant region.

The “variable domain” (variable domain of a light chain (V_{L}), variable region of a heavy chain (V_{H})) as used herein denotes each of the pair of light and heavy chains which are involved directly in binding the antibody to the target. The domains of variable human light and heavy chains have the same general structure and each domain comprises at least one complementary determining region (CDR), preferably three CDRs, which play a particularly important role in the binding specificity/affinity of the antibodies according to the invention and therefore provide a further object of the invention.

In one embodiment, the V_{H} of the anti-CD3-Fab of the present invention comprises SEQ ID NO: 21 (CDR1) or SEQ ID NO: 32 (CDR1), SEQ ID NO: 25 (CDR2) or SEQ ID NO: 33 (CDR2), and SEQ ID NO: 29 (CDR3) or SEQ ID NO: 34 (CDR3).

In one preferred embodiment, the V_{H} of the anti-CD3-Fab of the present invention comprises SEQ ID NO: 21 (CDR1), SEQ ID NO: 25 (CDR2), and SEQ ID NO: 29 (CDR3).

In one embodiment, the V_{H} of the anti-CD3-Fab of the present invention comprises the amino acid sequence of SEQ ID NO: 9 or SEQ ID NO: 10.

In one preferred embodiment, the V_{H} of the anti-CD3-Fab of the present invention comprises the amino acid sequence of SEQ ID NO: 9.

In one embodiment, the V_{L} of the anti-CD3-Fab of the present invention comprises SEQ ID NO: 22 (CDR1) or SEQ ID NO: 81 (CDR1), SEQ ID NO: 26 (CDR2) or SEQ ID NO: 82 (CDR2), and SEQ ID NO: 30 (CDR3) or SEQ ID NO: 83 (CDR3).

In one preferred embodiment, the V_{L} of the anti-CD3-Fab of the present invention comprises SEQ ID NO: 22 (CDR1), SEQ ID NO: 26 (CDR2), and SEQ ID NO: 30 (CDR3).

In one embodiment, the V_{L} of the anti-CD3-Fab of the present invention comprises the amino acid sequence of SEQ ID NO: 11 or SEQ IN NO: 12.

In one preferred embodiment, the V_{L} of the anti-CD3-Fab of the present invention comprises the amino acid sequence of SEQ ID NO: 11.

In one embodiment, the CH1 of the anti-CD3-Fab of the present invention comprises the amino acid sequence of SEQ ID NO: 18 or SEQ ID NO: 35.

In one preferred embodiment, the CH1 of the anti-CD3-Fab of the present invention comprises the amino acid sequence of SEQ ID NO: 18.

In one embodiment, the C_{κ} of the anti-CD3-Fab of the present invention comprises the amino acid sequence of SEQ ID NO: 17.

In one embodiment, the anti-CD3-Fab of the present invention comprises:
(1) a heavy chain region comprising a heavy chain variable (V_{H}) comprising SEQ ID NO: 21 (CDR1) or SEQ ID NO: 32 (CDR1), SEQ ID NO: 25 (CDR2) or SEQ ID NO: 33 (CDR2), and SEQ ID NO: 29 (CDR3) or SEQ ID NO: 34 (CDR3); and a heavy chain constant domain 1 (CH1) comprising the amino acid sequence of SEQ ID NO: 18 or SEQ ID NO: 35; and
(2) a light chain region comprising a light chain variable (V_{L}) comprising SEQ ID NO: 22 (CDR1) or SEQ ID NO: 81 (CDR1), SEQ ID NO: 26 (CDR2) or SEQ ID NO: 82 (CDR2), and SEQ ID NO: 30 (CDR3) or SEQ ID NO: 83 (CDR3); and a kappa constant light chain (C_{κ}) comprising the amino acid sequence of SEQ ID NO: 17.

In one preferred embodiment, the anti-CD3-Fab of the present invention comprises:
(1) a heavy chain region comprising a heavy chain variable (V_{H}) comprising SEQ ID NO: 21 (CDR1), SEQ ID NO: 25 (CDR2), and SEQ ID NO: 29 (CDR3); and a heavy chain constant domain 1 (CH1) comprising the amino acid sequence of SEQ ID NO: 18; and
(2) a light chain region comprising a light chain variable (V_{L}) comprising SEQ ID NO: 22 (CDR1), SEQ ID NO: 26 (CDR2), and SEQ ID NO: 30 (CDR3); and a kappa constant light chain (C_{κ}) comprising the amino acid sequence of SEQ ID NO: 17.

In one embodiment, the anti-CD3-Fab of the present invention comprises:
(1) a heavy chain region comprising a heavy chain variable (V_{H}) comprising the amino acid sequence of SEQ ID NO: 9 or SEQ ID NO: 10 and a heavy chain constant domain 1 (CH1) comprising the amino acid sequence of SEQ ID NO: 18 or SEQ ID NO: 35, and
(2) a light chain region comprising a light chain variable (V_{L}) comprising the amino acid sequence of SEQ ID NO: 11 or SEQ ID NO: 12 and a kappa constant light chain (C_{κ}) comprising the amino acid sequence of SEQ ID NO: 17.

In one embodiment, the anti-CD3-Fab of the present invention comprises:
(1) a heavy chain region comprising a heavy chain variable (V_{H}) comprising the amino acid sequence of SEQ ID NO: 9 and a heavy chain constant domain 1 (CH1) comprising the amino acid sequence of SEQ ID NO: 18,
(2) a light chain region comprising a light chain variable (V_{L}) comprising the amino acid sequence of SEQ ID NO: 11 and a kappa constant light chain (C_{κ}) comprising the amino acid sequence of SEQ ID NO: 17.

The Fc Region

In one embodiment, the bispecific molecule of the present invention further comprises a fragment crystallizable region (Fc).

The term “Fc” or “Fc region” is a term well known to the skilled artisan and is involved in complement activation, Clq binding, C3 activation and Fc receptor binding.

In one embodiment, the Fc region of the present invention is derived from human origin.

In one embodiment, the Fc region of the present invention is a human IgG1 Fc region or derived from a human IgG1 Fc region.

In one embodiment, the Fc region of the present invention comprises a first CH2CH3 region comprising a first CH2 domain and a first CH3 domain.

In one embodiment, the Fc region of the present invention comprises a second CH2′CH3′ region comprising a second CH2 domain (CH2′) and a second CH3 domain (CH3′).

In one embodiment, the Fc region of the present invention comprises a first constant region comprising a first constant domain 2 (CH2) and a first constant domain 3 (CH3).

In one embodiment, the Fc region of the present invention comprises a second constant region comprising a second constant domain 2 (CH2′) and a second constant domain 3 (CH3′).

In embodiments, the Fc region comprises a first CH2CH3 region and a second CH2′CH3′ region, wherein the first CH2CH3 region is covalently bound by two disulfide bridges to the second CH2′CH3′ region.

In one embodiment, the Fc region of the present invention comprises a hinge region. The term “hinge region” refers to a flexible amino acid stretch in the central part of the heavy chains of immunoglobulin antibodies, which links these 2 chains by disulfide bonds. Various hinge regions can be used in the bispecific molecules of the present invention, for example, to optimize certain characteristics. In an illustrative example, one or more amino acid substitutions, insertions, and/or deletions within a hinge region of a human IgG₁, IgG₂, IgG₃ or IgG₄ can be introduced to reduce the level or rate of fragmentation and/or aggregation.

In one embodiment, the Fc region of the present invention is engineered to comprise at least one amino acid substitution in the human IgG1 Fc region in its constant heavy chain domain 3 (CH3) to promote the heterodimerization through “knob-in-hole” technology (KiH). In this technique, through gene manipulation, a mutation is induced in a CH3 domain of two different Ig heavy chains, a hole structure is made in a CH3 domain of one Ig heavy chain, a knob structure is made the CH3 domain of the other Ig heavy chain, and two Ig heavy chains are induced to form a heterodimer (e.g., Carter, P., J. Immunol. Meth. 248 (2001) 7-15; Merchant, A. M., et al., Nat. Biotechnol. 16 (1998) 677-681; Zhu, Z., et al., Prot. Sci. 6 (1997) 781-788; Ridgway, J. B., et al., Prot. Eng. 9 (1996) 617-621; Atwell, S., et al., J. Mol. Biol. 270 (1997) 26-35).

For example, amino acid residues included in a hydrophobic core contributing to formation of the homodimer between human IgG1 heavy chain CH3 domains are Leu351, Thr366, Leu368, and Tyr407 according to EU numbering of the amino acid number of the antibody chain (Cunningham, Pflumm et al. 1969). In the knob-into-hole technique, with respect to residues positioned at a hydrophobic core in a CH3 domain interface, a hole structure is made in one heavy chain CH3 domain such that hydrophobic amino acid residues having a large side chain are substituted with hydrophobic amino acids having a small side chain (Thr366Ser, Leu368Ala, Tyr407Val), a knob structure is made in the other heavy chain CH3 domain such that hydrophobic amino acid residues having a small side chain are substituted with hydrophobic amino acids having a large side chain (Thr366Trp). When two mutation pairs, heavy chain constant region mutation pairs in which the first CH3 (Thr366Ser, Leu368Ala, and Tyr407Val) and the second CH3′ (Thr366Trp) are introduced to form the heterodimeric Fc.

In one embodiment, the Fc region of the present invention is derived from the human IgG1 Fc region and comprises the amino acid sequence of SEQ ID NO: 13 comprising in the first CH3 domain at least one of the following amino acid substitutions: Thr128 with serine, Leu130 with alanine, and Tyr169 with valine, which are corresponding to Thr366Ser, Leu368Ala and Tyr407Val of the human IgG1 heavy chain respectively according to EU numbering of the amino acid number of the antibody chain.

In one embodiment, the Fc region of the present invention is derived from the human IgG1 Fc region and comprises the amino acid sequence of SEQ ID NO: 13 comprising in the first CH3 domain of the human IgG1 Fc region, amino acid substitutions of Thr128 with serine, Leu130 with alanine, and Tyr169 with valine, which are corresponding to Thr366, Leu368 and Tyr407 of the human IgG1 heavy chain respectively according to EU numbering of the amino acid number of the antibody chain.

In one embodiment, the Fc region of the present invention is derived from the human IgG1 Fc region and comprises the amino acid sequence of SEQ ID NO: 16 in the second CH3 domain (CH3′) an amino acid substitution of Thr146 with tryptophan, which corresponds to Thr366 of the human IgG1 heavy chain respectively according to EU numbering of the amino acid number of the antibody chain.

In one embodiment, the Fc region of the present invention comprises one or more mutations to modulate Fc receptor-based function of the Fc region. In one embodiment, the Fc region of the present invention comprises one or more mutations to modulate FcγR-based effector function of the Fc region. In one embodiment, the Fc region of the present invention is derived from the human IgG1 Fc region and comprises the amino acid sequence of SEQ ID NO: 13 comprising in the first CH2 domain an amino acid substitution of Asn59 with alanine, which corresponds to Asn297 of the human IgG1 heavy chain respectively according to EU numbering of the amino acid number of the antibody chain. In one embodiment, the Fc region of the present invention is derived from the human IgG1 Fc region and comprises the amino acid sequence of SEQ ID NO: 16 comprising in the second CH2 domain (CH2′) an amino acid substitution of Asn77 with alanine, which corresponds to Asn297 of the human IgG1 heavy chain respectively according to EU numbering of the amino acid number of the antibody chain.

In one embodiment, the Fc region of the present invention comprises a first constant region (CH2CH3) comprising a first constant domain 2 (CH2) and a first constant domain 3 (CH3).

In one embodiment, the Fc region of the present invention comprises:
(1) a first constant region CH2CH3 comprising a first constant domain 2 (CH2) and a first constant domain 3 (CH3); and
(2) a second constant region CH2′CH3′ comprising a second constant domain 2 (CH2′) and a second constant domain 3 (CH3′).

In one embodiment, the CH2CH3 of the Fc region of the present invention comprises the amino acid sequence selected from a group consisting of SEQ ID NO: 13, SEQ ID NO: 14 and SEQ ID NO: 15.

In one embodiment, the CH2CH3 of the Fc region of the present invention comprises the amino acid sequence of SEQ ID NO: 13.

In one embodiment, the CH2′ CH3′ of the Fc region of the present invention comprises the amino acid sequence of SEQ ID NO: 16.

In one embodiment, the Fc region of the present invention comprises:
(1) a first constant region comprising a first constant domain 2 (CH2) and a first constant domain 3 (CH3), wherein the CH2CH3 comprises the amino acid sequence selected from a group consisting of SEQ ID NO: 13, SEQ ID NO: 14 and SEQ ID NO: 15; and
(2) a second constant region comprising a second constant domain 2 (CH2′) and a second constant domain 3 (CH3′), wherein the CH2′CH3′ comprises the amino acid sequence of SEQ ID NO: 16.

In one embodiment, the Fc region of the present invention comprises:
(1) a first constant region comprising a first constant domain 2 (CH2) and a first constant domain 3 (CH3), wherein the CH2CH3 comprises the amino acid sequence of SEQ ID NO: 13; and
(2) a second constant region comprising a second constant domain 2 (CH2′) and a second constant domain 3 (CH3′), wherein the CH2′CH3′ comprises the amino acid sequence of SEQ ID NO: 16.

In one embodiment, the Fc region of the present invention is connected to the anti-CD3-Fab of the present invention between the CH1 of the anti-CD3-Fab and the CH2 of the Fc region to form a CH1CH2CH3 domain.

In one embodiment, the CH1CH2CH3 of the present invention comprises the amino acid sequence selected from a group consisting of SEQ ID NO: 37, SEQ ID NO: 38 and SEQ ID NO: 39.

In one preferred embodiment, the CH1CH2CH3 of the present invention comprises the amino acid sequence of SEQ ID NO: 37.

Formats of the Bispecific Molecules

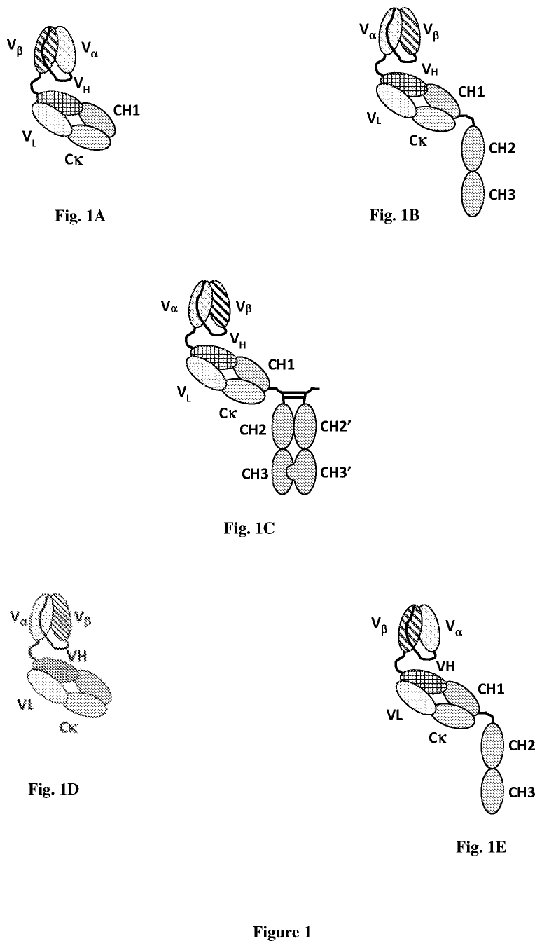

According to the present invention, the CD3 binding part, the Survivin binding part and the Fc region can be formatted in various orientations. To assist understanding, five exemplary embodiments of bispecific molecules are illustrated in FIGS. 1A-1E.

With reference to FIG. 1A, one exemplary embodiment of a bispecific molecule comprises (1) a single-chain soluble T cell receptor (sTCR) which binds to a complex of the Survivin peptide and the HLA-A2 molecule and (2) an antigen binding fragment (Fab) which binds to human CD3 (anti-CD3-Fab). The sTCR comprises a sTCR variable beta region (V_{α}) and a sTCR variable alpha region (V_{β}), where the V_{α} and the V_{β} are connected via a first peptide linker (L1). The anti-CD3-Fab comprises a heavy chain variable (V_{H}), a heavy chain constant domain 1 (CH1), a light chain variable (V_{L}) and a kappa constant light chain (C_{κ}) The sTCR and the anti-CD3-Fab are connected via a second peptide linker (L2) connecting the V_{β} of the sTCR and the V_{H} of the anti-CD3-Fab. The bispecific molecule is in the form of V_{α}-L1-V_{β}-L2-anti-CD3-Fab.

With reference to FIG. 1B, one exemplary embodiment of a bispecific molecule comprises (1) a single-chain soluble T cell receptor (sTCR) which binds to a complex of the Survivin peptide and the HLA-A2 molecule, (2) an antigen binding fragment (Fab) which binds to human CD3 (anti-CD3-Fab) and (3) a fragment crystallizable region (Fc). The sTCR, the anti-CD3-Fab and the Fc region are connected in the order of sTCR-anti-CD3-Fab-Fc. The sTCR comprises a sTCR variable beta region (V_{β}) and a sTCR variable alpha region (V_{α}), where the V_{β} and the V_{α} are connected via a first peptide linker (L1). The anti-CD3-Fab comprises a heavy chain variable (V_{H}), a heavy chain constant domain 1 (CH1), a light chain variable (V_{L}) and a kappa constant light chain (C_{κ}). The sTCR and the anti-CD3-Fab are connected via a second linker (L2) between the V_{α} of the sTCR and V_{H} of the anti-CD3-Fab. The Fc region comprises a constant domain 2 (CH2) and a constant domain 3 (CH3). The anti-CD3-Fab and the Fc region are enclosed in a format of a half-body of an antibody. The bispecific molecule is in the form of V_{β}-L1-V_{α}-L2-anti-CD3-Fab-Fc.

With reference to FIG. 1C, one exemplary embodiment of a bispecific molecule comprises (1) a single-chain soluble T cell receptor (sTCR) which binds to a complex of the Survivin peptide and the HLA-A2 molecule, (2) an antigen binding fragment (Fab) which binds to human CD3 (anti-CD3-Fab) and (3) a fragment crystallizable region (Fc). The sTCR, the anti-CD3-Fab and the Fc region are connected in the order of sTCR-anti-CD3-Fab-Fc. The sTCR comprises a sTCR variable beta region (V_{β}) and a sTCR variable alpha region (V_{α}), where the V_{β} and the V_{α} are connected via a first peptide linker (L1). The anti-CD3-Fab comprises a heavy chain variable (V_{H}), a heavy chain constant domain 1 (CH1), a light chain variable (V_{L}) and a kappa constant light chain (C_{κ}). The sTCR and the anti-CD3-Fab are connected via a second peptide linker (L2) between the V_{α} and V_{H}. The Fc region comprises a first constant region comprising a first constant domain 2 (CH2) and a first constant domain 3 (CH3) and a second constant region comprising a second constant domain 2 (CH2′) and a second constant domain 3 (CH3′). The first CH3 and the second CH3 are engineered for heterodimerization through “knob-in-hole” technology. The bispecific molecule is in the form of V_{β}-L1-V_{α}-L2-anti-CD3-Fab-Fc.

With reference to FIG. 1D, one exemplary embodiment of a bispecific molecule comprises (1) a single-chain soluble T cell receptor (sTCR) which binds to a complex of the Survivin peptide and the HLA-A2 molecule and (2) an antigen binding fragment (Fab) which binds to human CD3 (anti-CD3-Fab). The sTCR comprises a sTCR variable beta region (V_{β}) and a sTCR variable alpha region (V_{α}), where the V_{β} and the V_{α} are connected via a first peptide linker (L1). The anti-CD3-Fab comprises a heavy chain variable (V_{H}), a heavy chain constant domain 1 (CH1), a light chain variable (V_{L}) and a kappa constant light chain (C_{κ}). The sTCR and the anti-CD3-Fab are connected via a second peptide linker (L2) connecting the V_{α} of the sTCR and the V_{H} of the anti-CD3-Fab. The bispecific molecule is in the form of V_{β}-L1-V_{α}-L2-anti-CD3-Fab.

With reference to FIG. 1E, one exemplary embodiment of a bispecific molecule comprises (1) a single-chain soluble T cell receptor (sTCR) which binds to a complex of the Survivin peptide and the HLA-A2 molecule, (2) an antigen binding fragment (Fab) which binds to human CD3 (anti-CD3-Fab) and (3) a fragment crystallizable region (Fc). The sTCR, the anti-CD3-Fab and the Fc region are connected in the order of sTCR-anti-CD3-Fab-Fc. The sTCR comprises a sTCR variable beta region (V_{α}) and a sTCR variable alpha region (V_{β}), where the V_{α} and the V_{β} are connected via a first peptide linker (L1). The anti-CD3-Fab comprises a heavy chain variable (V_{H}), a heavy chain constant domain 1 (CH1), a light chain variable (V_{L}) and a kappa constant light chain (C_{κ}) The sTCR and the anti-CD3-Fab are connected via a second linker (L2) between the V_{β} of the sTCR and V_{H} of the anti-CD3-Fab. The Fc region comprises a constant domain 2 (CH2) and a constant domain 3 (CH3). The anti-CD3-Fab and the Fc region are enclosed in a format of a half-body of an antibody. The bispecific molecule is in the form of V_{α}-L1-V_{β}-L2-anti-CD3-Fab-Fc.

The following specific embodiments of the present invention are listed:

In one embodiment, the present invention provides a bispecific molecule comprising:
a) a single-chain soluble T cell receptor (sTCR), which binds to a complex of the peptide Survivin, wherein the complex comprises the amino acid sequence of SEQ ID NO: 40 and the HLA-A2 molecule, wherein the sTCR comprises:
   (1) a sTCR variable beta region (V_{β}) comprising SEQ ID NO: 20 (CDR1), SEQ ID NO: 24 (CDR2), and SEQ ID NO: 28 (CDR3) or SEQ ID NO: 31 (CDR3), and
   (2) a sTCR variable alpha region (V_{α}) comprising SEQ ID NO: 19 (CDR1), SEQ ID NO: 23 (CDR2), and SEQ ID NO: 27 (CDR3);
      wherein the V_{α} and V_{β} regions of the sTCR are connected via a first peptide linker (L1);
   b) an antigen binding fragment (Fab) which binds to human CD3 (anti-CD3-Fab), wherein the anti-CD3-Fab comprises:
      (1) a heavy chain region comprising a heavy chain variable (V_{H}) comprising SEQ ID NO: 21 (CDR1) or SEQ ID NO: 32 (CDR1), SEQ ID 25 (CDR2) or SEQ ID NO: 33 (CDR2), and SEQ ID NO: 29 (CDR3) or SEQ ID NO: 34 (CDR3); and a heavy chain constant domain 1 (CH1) comprising the amino acid sequence of SEQ ID NO: 18 or SEQ ID NO: 35 and,
      (2) a light chain region comprising a light chain variable (V_{L}) comprising SEQ ID NO: 22 (CDR1) or SEQ ID NO: 81 (CDR1), SEQ ID NO: 26 (CDR2) or SEQ ID NO: 82 (CDR2), and SEQ ID NO: 30 (CDR3) or SEQ ID NO: 83 (CDR3); and a kappa constant light chain (C_{κ}) comprising the amino acid sequence of SEQ ID NO: 17,
         wherein the V_{β} of the sTCR and the V_{H} of the anti-CD3-Fab are connected via a second peptide linker (L2);

wherein the bispecific molecule is in the form of

V_{β}-L1-V_{β}-L2-V_{H}-CH1 V_{L}-Cκ.

In one embodiment, the present invention provides a bispecific molecule comprising:
a) a single-chain soluble T cell receptor (sTCR), which binds to a complex of the peptide Survivin, wherein the complex comprises the amino acid sequence of SEQ ID NO: 40 and the HLA-A2 molecule, wherein the sTCR comprises:
   (1) a sTCR variable beta region (V_{β}) comprising the amino acid sequence selected from a group consisting of SEQ ID NO: 2, SEQ ID NO: 3, SEQ ID NO: 4 and SEQ ID NO: 5, and
   (2) a sTCR variable alpha region (V_{α}) comprising the amino acid sequence selected from a group consisting of SEQ ID NO: 6, SEQ ID NO: 7 and SEQ ID NO: 8,
      wherein the V_{α} and V_{β} regions of the sTCR are connected via a first peptide linker (L1);
b) an antigen binding fragment (Fab) which binds to human CD3 (anti-CD3-Fab), wherein the anti-CD3-Fab comprises:
   (1) a heavy chain region comprising a heavy chain variable (V_{H}) comprising the amino acid sequence of SEQ ID NO: 9 or SEQ ID NO: 10; and a heavy chain constant domain 1 (CH1) comprising the amino acid sequence of SEQ ID NO: 18 or SEQ ID NO: 35, and
   (2) a light chain region comprising a light chain variable (V_{L}) comprising the amino acid sequence of SEQ ID NO: 11 or SEQ ID NO: 12; and a kappa constant light chain (C_{κ}) comprising the amino acid sequence of SEQ ID NO: 17,
      wherein the V_{β} of the sTCR and the V_{H} of the anti-CD3-Fab are connected via a second peptide linker (L2),

wherein the bispecific molecule is in the form of

V_{α}-L1-V_{β}-L2-V_{H}-CH1 V_{L}-Cκ.

In one embodiment, the present invention provides a bispecific molecule comprising:
a) a single-chain soluble T cell receptor (sTCR), which binds to a complex of the peptide Survivin, wherein the complex comprises the amino acid sequence of SEQ ID NO: 40 and the HLA-A2 molecule, wherein the sTCR comprises:
   (1) a sTCR variable beta region (V_{β}) comprising SEQ ID NO: 20 (CDR1), SEQ ID NO: 24 (CDR2), and SEQ ID NO: 28 (CDR3) or SEQ ID NO: 31 (CDR3), and
   (2) a sTCR variable alpha region (V_{α}) comprising SEQ ID NO: 19 (CDR1), SEQ ID NO: 23 (CDR2), and SEQ ID NO: 27 (CDR3);
      wherein the V_{β} and V_{α} regions of the sTCR are connected via a first peptide linker (L1);
b) an antigen binding fragment (Fab) which binds to human CD3 (anti-CD3-Fab), wherein the anti-CD3-Fab comprises:
   (1) a heavy chain region comprising a heavy chain variable (V_{H}) comprising SEQ ID NO: 21 (CDR1) or SEQ ID NO: 32 (CDR1), SEQ ID NO: 25 (CDR2) or SEQ ID NO: 33 (CDR2), and SEQ ID NO: 29 (CDR3) or SEQ ID NO: 34 (CDR3); and a heavy chain constant domain 1 (CH1) comprising the amino acid sequence of SEQ ID NO: 18 or SEQ ID NO: 35, and
   (2) a light chain region comprising a light chain variable (V_{L}) comprising SEQ ID NO: 22 (CDR1) or SEQ ID NO: 81 (CDR1), SEQ ID NO: 26 (CDR2) or SEQ ID NO: 82 (CDR2), and SEQ ID NO: 30 (CDR3) or SEQ ID NO: 83 (CDR3); and a kappa constant light chain (C_{κ}) comprising the amino acid sequence of SEQ ID NO: 17,
      wherein the V_{α} of the sTCR and the V_{H} of the anti-CD3-Fab are connected via a second peptide linker (L2);

wherein the bispecific molecule is in the form of

V_{β}-L1-V_{α}-L2-V_{H}-CH1 V_{L}-Cκ.

In one embodiment, the present invention provides a bispecific molecule comprising:
a) a single-chain soluble T cell receptor (sTCR), which binds to a complex of the peptide Survivin, wherein the complex comprises the amino acid sequence of SEQ ID NO: 40 and the HLA-A2 molecule, wherein the sTCR comprises:
   (1) a sTCR variable beta region (V_{β}) comprising the amino acid sequence selected from a group consisting of SEQ ID NO: 2, SEQ ID NO: 3, SEQ ID NO: 4 and SEQ ID NO: 5, and
   (2) a sTCR variable alpha region (V_{α}) comprising the amino acid sequence selected from a group consisting of SEQ ID NO: 6, SEQ ID NO: 7 and SEQ ID NO: 8,
      wherein the V_{β} and V_{α} regions of the sTCR are connected via a first peptide linker (L1);
b) an antigen binding fragment (Fab) which binds to human CD3 (anti-CD3-Fab), wherein the anti-CD3-Fab comprises:
   (1) a heavy chain region comprising a heavy chain variable (V_{H}) comprising the amino acid sequence of SEQ ID NO: 9 or SEQ ID NO: 10; and a heavy chain constant domain 1 (CH1) comprising the amino acid sequence of SEQ ID NO: 18 or SEQ ID NO: 35, and
   (2) a light chain region comprising a light chain variable (V_{L}) comprising the amino acid sequence of SEQ ID NO: 11 or SEQ ID NO: 12; and a kappa constant light chain (C_{κ}) comprising the amino acid sequence of SEQ ID NO: 17,
      wherein the V_{α} of the sTCR and the V_{H} of the anti-CD3-Fab are connected via a second peptide linker (L2),

wherein the bispecific molecule is in the form of

V_{β}-L1-V_{α}-L2-V_{H}-CH1 V_{L}-Cκ.

In one embodiment, the present invention provides a bispecific molecule comprising:
a) a single-chain soluble T cell receptor (sTCR), which binds to a complex of the peptide Survivin, wherein the complex comprises the amino acid sequence of SEQ ID NO: 40 and the HLA-A2 molecule, wherein the sTCR comprises:
   (1) a sTCR variable beta region (V_{β}) comprising SEQ ID NO: 20 (CDR1), SEQ ID NO: 24 (CDR2), and SEQ ID NO: 28 (CDR3) or SEQ ID NO: 31 (CDR3), and
   (2) a sTCR variable alpha region (V_{α}) comprising SEQ ID NO: 19 (CDR1), SEQ ID NO: 23 (CDR2), and SEQ ID NO: 27 (CDR3),
      wherein the V_{β} and V_{α} regions of the sTCR are connected via a first peptide linker (L1);
b) an antigen binding fragment (Fab) which binds to human CD3 (anti-CD3-Fab), wherein the anti-CD3-Fab comprises:
   (1) a heavy chain region comprising a heavy chain variable (V_{H}) comprising SEQ ID NO: 21 (CDR1) or SEQ ID NO: 32 (CDR1), SEQ ID NIO: 25 (CDR2) or SEQ ID NO: 33 (CDR2), and SEQ ID NO: 29 (CDR3) or SEQ ID NO: 34 (CDR3); and a heavy chain constant domain 1 (CH1) comprising the amino acid sequence of SEQ ID NO: 18 or SEQ ID NO: 35, and
   (2) a light chain region comprising a light chain variable (V_{L}) comprising SEQ ID NO: 22 (CDR1) or SEQ ID NO: 81 (CDR1), SEQ ID NO: 26 (CDR2) or SEQ ID NO: 82 (CDR2), and SEQ ID NO: 30 (CDR3) or SEQ ID NO: 83 (CDR3); and a kappa constant light chain (C_{κ}) comprising the amino acid sequence of SEQ ID NO: 17,
      wherein the V_{α} of the sTCR and the V_{H} of the anti-CD3-Fab are connected via a second peptide linker (L2); and
c) a fragment crystallizable region (Fc) comprising a constant domain 2 (CH2) and a constant domain 3 (CH3), wherein the CH2CH3 comprises the amino acid sequence selected from a group consisting of SEQ ID NO: 13, SEQ ID NO: 14 and SEQ ID NO: 15;

wherein the bispecific molecule is in the form of

V_{β}-L1-V_{α}-L2-V_{H}-CH1CH2CH3V_{L}-Cκ.

In one embodiment, the present invention provides a bispecific molecule comprising:
a) a single-chain soluble T cell receptor (sTCR), which binds to a complex of the peptide Survivin, wherein the complex comprises the amino acid sequence of SEQ ID NO: 40 and the HLA-A2 molecule, wherein the sTCR comprises:
   (1) a sTCR variable beta region (V_{β}) comprising the amino acid sequence selected from a group consisting of SEQ ID NO: 2, SEQ ID NO: 3, SEQ ID NO: 4 and SEQ ID NO: 5, and
   (2) a sTCR variable alpha region (V_{α}) comprising the amino acid sequence selected from a group consisting of SEQ ID NO: 6, SEQ ID NO: 7 and SEQ ID NO: 8,
      wherein the V_{β} and V_{α} regions of the sTCR are connected via a first peptide linker (L1);
b) an antigen binding fragment (Fab) which binds to human CD3 (anti-CD3-Fab), wherein the anti-CD3-Fab comprises:
   (1) a heavy chain region comprising a heavy chain variable (V_{H}) comprising the amino acid sequence of SEQ ID NO: 9 or SEQ ID NO: 10; a heavy chain constant domain 1 (CH1) comprising the amino acid sequence of SEQ ID NO: 18 or SEQ ID NO: 35, and
   (2) a light chain region comprising a light chain variable (V_{L}) comprising the amino acid sequence of SEQ ID NO: 11 or SEQ ID NO: 12; and a kappa constant light chain (C_{κ}) comprising the amino acid sequence of SEQ ID NO: 17,
      wherein the V_{α} of the sTCR and the V_{H} of the anti-CD3-Fab are connected via a second peptide linker (L2); and
c) a fragment crystallizable region (Fc) comprising a constant domain 2 (CH2) and a constant domain 3 (CH3), wherein the CH2CH3 comprises the amino acid sequence selected from a group consisting of SEQ ID NO: 13, SEQ ID NO: 14 and SEQ ID NO: 15;
wherein the bispecific molecule is in the form of

V_{β}-L1-V_{α}-L2-V_{H}-CH1CH2CH3V_{L}-Cκ.

In one embodiment, the present invention provides a bispecific molecule comprising:
a) a single-chain soluble T cell receptor (sTCR), which binds to a complex of the peptide Survivin, wherein the complex comprises the amino acid sequence of SEQ ID NO: 40 and the HLA-A2 molecule, wherein the sTCR comprises:
   (1) a sTCR variable beta region (V_{β}) comprising SEQ ID NO: 20 (CDR1), SEQ ID NO: 24 (CDR2), and SEQ ID NO: 28 (CDR3) or SEQ ID NO: 31 (CDR3), and
   (2) a sTCR variable alpha region (V_{α}) comprising SEQ ID NO: 19 (CDR1), SEQ ID NO: 23 (CDR2), and SEQ ID NO: 27 (CDR3),
      wherein the V_{α} and V_{β} regions of the sTCR are connected via a first peptide linker (L1);
b) an antigen binding fragment (Fab) which binds to human CD3 (anti-CD3-Fab), wherein the anti-CD3-Fab comprises:
   (1) a heavy chain region comprising a heavy chain variable (V_{H}) comprising SEQ ID NO: 21 (CDR1) or SEQ ID NO: 32 (CDR1), SEQ ID NIO: 25 (CDR2) or SEQ ID NO: 33 (CDR2), and SEQ ID NO: 29 (CDR3) or SEQ ID NO: 34 (CDR3); and a heavy chain constant domain 1 (CH1) comprising the amino acid sequence of SEQ ID NO: 18 or SEQ ID NO: 35, and
   (2) a light chain region comprising a light chain variable (V_{L}) comprising SEQ ID NO: 22 (CDR1) or SEQ ID NO: 81 (CDR1), SEQ ID NO: 26 (CDR2) or SEQ ID NO: 82 (CDR2), and SEQ ID NO: 30 (CDR3) or SEQ ID NO: 83 (CDR3); and a kappa constant light chain (C_{κ}) comprising the amino acid sequence of SEQ ID NO: 17,
      wherein the V_{β} of the sTCR and the V_{H} of the anti-CD3-Fab are connected via a second peptide linker (L2); and
c) a fragment crystallizable region (Fc) comprising a constant domain 2 (CH2) and a constant domain 3 (CH3), wherein the CH2CH3 comprises the amino acid sequence selected from a group consisting of SEQ ID NO: 13, SEQ ID NO: 14 and SEQ ID NO: 15;

wherein the bispecific molecule is in the form of

V_{α}-L1-V_{β}-L2-V_{H}-CH1CH2CH3V_{L}-Cκ.

In one embodiment, the present invention provides a bispecific molecule comprising:
a) a single-chain soluble T cell receptor (sTCR), which binds to a complex of the peptide Survivin, wherein the complex comprises the amino acid sequence of SEQ ID NO: 40 and the HLA-A2 molecule, wherein the sTCR comprises:
   (1) a sTCR variable beta region (V_{β}) comprising the amino acid sequence selected from a group consisting of SEQ ID NO: 2, SEQ ID NO: 3, SEQ ID NO: 4 and SEQ ID NO: 5, and
   (2) a sTCR variable alpha region (V_{α}) comprising the amino acid sequence selected from a group consisting of SEQ ID NO: 6, SEQ ID NO: 7 and SEQ ID NO: 8,
      wherein the V_{α} and V_{β} regions of the sTCR are connected via a first peptide linker (L1);
b) an antigen binding fragment (Fab) which binds to human CD3 (anti-CD3-Fab), wherein the anti-CD3-Fab comprises:
   (1) a heavy chain region comprising a heavy chain variable (V_{H}) comprising the amino acid sequence of SEQ ID NO: 9 or SEQ ID NO: 10; and a heavy chain constant domain 1 (CH1) comprising the amino acid sequence of SEQ ID NO: 18 or SEQ ID NO: 35, and
   (2) a light chain region comprising a light chain variable (V_{L}) comprising the amino acid sequence of SEQ ID NO: 11 or SEQ ID NO: 12; and a kappa constant light chain (C_{κ}) comprising the amino acid sequence of SEQ ID NO: 17,
      wherein the V_{β} of the sTCR and the V_{H} of the anti-CD3-Fab are connected via a second peptide linker (L2); and
c) a fragment crystallizable region (Fc) comprising a constant domain 2 (CH2) and a constant domain 3 (CH3), wherein the CH2CH3 comprises the amino acid sequence selected from a group consisting of SEQ ID NO: 13, SEQ ID NO: 14 and SEQ ID NO: 15;
   wherein the bispecific molecule is in the form of

V_{α}-L1-V_{β}-L2-V_{H}-CH1CH2CH3V_{L}-Cκ.

In one embodiment, the present invention provides a bispecific molecule comprising:
a) a single-chain soluble T cell receptor (sTCR), which binds to a complex of the peptide Survivin, wherein the complex comprises the amino acid sequence of SEQ ID NO: 40 and the HLA-A2 molecule, wherein the sTCR comprises:
   (1) a sTCR variable beta region (V_{β}) comprising SEQ ID NO: 20 (CDR1), SEQ ID NO: 24 (CDR2), and SEQ ID NO: 28 (CDR3) or SEQ ID NO: 31 (CDR3), and
   (2) a sTCR variable alpha region (V_{α}) comprising SEQ ID NO: 19 (CDR1), SEQ ID NO: 23 (CDR2), and SEQ ID NO: 27 (CDR3),
      wherein the V_{β} and V_{α} regions of the sTCR are connected via a first peptide linker (L1);
b) an antigen binding fragment (Fab) which binds to human CD3 (anti-CD3-Fab), wherein the anti-CD3-Fab comprises:
   (1) a heavy chain variable (V_{H}) comprising SEQ ID NO: 21 (CDR1) or SEQ ID NO: 32 (CDR1), SEQ ID NO: 25 (CDR2) or SEQ ID NO: 33 (CDR2), and SEQ ID NO: 29 (CDR3) or SEQ ID NO: 34 (CDR3); and a heavy chain constant domain 1 (CH1) comprising the amino acid sequence of SEQ ID NO: 18 or SEQ ID NO: 35, and
   (2) a light chain region comprising a light chain variable (V_{L}) comprising SEQ ID NO: 22 (CDR1) or SEQ ID NO: 81 (CDR1), SEQ ID NO: 26 (CDR2) or SEQ ID NO: 82 (CDR2), and SEQ ID NO: 30 (CDR3) or SEQ ID NO: 83 (CDR3); and a kappa constant light chain (C_{κ}) comprising the amino acid sequence of SEQ ID NO: 17,
      wherein the V_{α} of the sTCR and the V_{H} of the anti-CD3-Fab are connected via a second peptide linker (L2); and
c) a fragment crystallizable region (Fc) comprising:
   (1) a first constant region comprising a first constant domain 2 (CH2) and a first constant domain 3 (CH3), wherein the first constant region (CH2CH3) comprises the amino acid sequence selected from a group consisting of SEQ ID NO: 13, SEQ ID NO: 14 and SEQ ID NO: 15, and
   (2) a second constant region comprising a second constant domain 2 (CH2′) and a second constant domain 3 (CH3′), wherein the second constant region (CH2′CH3′) comprises the amino acid sequence of SEQ ID NO: 16,

wherein the bispecific molecule is in the form of

V_{β}-L1-V_{α}-L2-V_{H}-CH1CH2CH3V_{L}-CκCH2′CH3′.

In one embodiment, the present invention provides a bispecific molecule comprising:
a) a single-chain soluble T cell receptor (sTCR), which binds to a complex of the peptide Survivin, wherein the complex comprises the amino acid sequence of SEQ ID NO: 40 and the HLA-A2 molecule, wherein the sTCR comprises:
   (1) a sTCR variable beta region (V_{β}) comprising SEQ ID NO: 20 (CDR1), SEQ ID NO: 24 (CDR2), and SEQ ID NO: 28 (CDR3), and
   (2) a sTCR variable alpha region (V_{α}) comprising SEQ ID NO: 19 (CDR1), SEQ ID NO: 23 (CDR2), and SEQ ID NO: 27 (CDR3),
      wherein the V_{β} and V_{α} regions of the sTCR are connected via a first peptide linker (L1);
b) an antigen binding fragment (Fab) which binds to human CD3 (anti-CD3-Fab), wherein the anti-CD3-Fab comprises:
   (1) a heavy chain variable (V_{H}) comprising SEQ ID NO: 21 (CDR1), SEQ ID NO: 25 (CDR2), and SEQ ID NO: 29 (CDR3); and a heavy chain constant domain 1 (CH1) comprising the amino acid sequence of SEQ ID NO: 18, and
   (2) a light chain region comprising a light chain variable (V_{L}) comprising SEQ ID NO: 22 (CDR1), SEQ ID NO: 26 (CDR2), and SEQ ID NO: 30 (CDR3); and a kappa constant light chain (C_{κ}) comprising the amino acid sequence of SEQ ID NO: 17,
      wherein the V_{α} of the sTCR and the V_{H} of the anti-CD3-Fab are connected via a second peptide linker (L2);
c) a fragment crystallizable region (Fc) comprising:
   (1) a first constant region comprising a first constant domain 2 (CH2) and a first constant domain 3 (CH3), wherein the first constant region (CH2CH3) comprises the amino acid sequence of SEQ ID NO: 13; and
   (2) a second constant region comprising a second constant domain 2 (CH2′) and a second constant domain 3 (CH3′), wherein the second constant region (CH2′CH3′) comprises the amino acid sequence of SEQ ID NO: 16;

wherein the bispecific molecule is in the form of

V_{β}-L1-V_{α}-L2-V_{H}-CH1CH2CH3V_{L}-CκCH2′CH3′.

In one embodiment, the present invention provides a bispecific molecule comprising:
a) a single-chain soluble T cell receptor (sTCR), which binds to a complex of the peptide Survivin, wherein the complex comprises the amino acid sequence of SEQ ID NO: 40 and the HLA-A2 molecule, wherein the sTCR comprises:
   (1) a sTCR variable beta region (V_{β}) comprising the amino acid sequence selected from a group consisting of SEQ ID NO: 2, SEQ ID NO: 3, SEQ ID NO: 4 and SEQ ID NO: 5, and
   (2) a sTCR variable alpha region (V_{α}) comprising the amino acid sequence selected from a group consisting of SEQ ID NO: 6, SEQ ID NO: 7 and SEQ ID NO: 8,
      wherein the V_{β} and V_{α} regions of the sTCR are connected via a first peptide linker (L1);
b) an antigen binding fragment (Fab) which binds to human CD3 (anti-CD3-Fab), wherein the anti-CD3-Fab comprises:
   (1) a heavy chain variable (V_{H}) comprising the amino acid sequence of SEQ ID NO: 9 or SEQ ID NO: 10, and a heavy chain constant domain 1 (CH1) comprising the amino acid sequence of SEQ ID NO: 18 or SEQ ID NO: 35, and
   (2) a light chain region comprising a light chain variable (V_{L}) comprising the amino acid sequence of SEQ ID NO: 11 or SEQ ID NO: 12; and a kappa constant light chain (C_{κ}) comprising the amino acid sequence of SEQ ID NO: 17,
      wherein the V_{α} of the sTCR and the V_{H} of the anti-CD3-Fab are connected via a second peptide linker (L2);
c) a fragment crystallizable region (Fc) comprising:
   (1) a first constant region comprising a first constant domain 2 (CH2) and a first constant domain 3 (CH3), wherein the first constant region (CH2CH3) comprises the amino acid sequence selected from a group consisting of SEQ ID NO: 13, SEQ ID NO: 14 and SEQ ID NO: 15; and
   (2) a second constant region comprising a second constant domain 2 (CH2′) and a second constant domain 3 (CH3′), wherein the second constant region (CH2′CH3′) comprises the amino acid sequence of SEQ ID NO: 16;

wherein the bispecific molecule is in the form of

V_{β}-L1-V_{α}-L2-V_{H}-CH1CH2CH3V_{L}-CκCH2′CH3′.

In one embodiment, the present invention provides a bispecific molecule comprising:
a) a single-chain soluble T cell receptor (sTCR), which binds to a complex of the peptide Survivin, wherein the complex comprises the amino acid sequence of SEQ ID NO: 40 and the HLA-A2 molecule, wherein the sTCR comprises:
   (1) a sTCR variable beta region (V_{β}) comprising the amino acid sequence of SEQ ID NO: 2, and
   (2) a sTCR variable alpha region (V_{α}) comprising the amino acid sequence of SEQ ID NO: 6,
      wherein the V_{β} and V_{α} regions of the sTCR are connected via a first peptide linker (L1);
b) an antigen binding fragment (Fab) which binds to human CD3 (anti-CD3-Fab), wherein the anti-CD3-Fab comprises:
   (1) a heavy chain variable (V_{H}) comprising the amino acid sequence of SEQ ID NO: 9, and a heavy chain constant domain 1 (CH1) comprising the amino acid sequence of SEQ ID NO: 18, and
   (2) a light chain region comprising a light chain variable (V_{L}) comprising the amino acid sequence of SEQ ID NO: 11; and a kappa constant light chain (C_{κ}) comprising the amino acid sequence of SEQ ID NO: 17,
      wherein the V_{α} of the sTCR and the V_{H} of the anti-CD3-Fab are connected via a second peptide linker (L2);
c) a fragment crystallizable region (Fc) comprising:
   (1) a first constant region comprising a first constant domain 2 (CH2) and a first constant domain 3 (CH3), wherein the first constant region (CH2CH3) comprises the amino acid sequence of SEQ ID NO: 13; and
   (2) a second constant region comprising a second constant domain 2 (CH2′) and a second constant domain 3 (CH3′), wherein the second constant region (CH2′CH3′) comprises the amino acid sequence of SEQ ID NO: 16;

wherein the bispecific molecule is in the form of

V_{β}-L1-V_{α}-L2-V_{H}-CH1CH2CH3V_{L}-CκCH2′CH3′.

In one embodiment, the present invention provides a bispecific molecule which binds to both human CD3 and a complex of the peptide Survivin, wherein the bispecific molecule comprises:
a) a first heavy chain region comprising
   1) a single-chain soluble T cell receptor (sTCR) which binds to a complex of the peptide Survivin, wherein the complex comprises the amino acid sequence of SEQ ID NO: 40 and the HLA-A2 molecule, wherein the sTCR comprises:
      i. a sTCR variable beta region (V_{β}) comprising SEQ ID NO: 20 (CDR1), SEQ ID NO: 24 (CDR2), and SEQ ID NO: 28 (CDR3) or SEQ ID NO: 31 (CDR3); and
      ii. a sTCR variable alpha region (V_{α}) comprising SEQ ID NO: 19 (CDR1), SEQ ID NO: 23 (CDR2), and SEQ ID NO: 27 (CDR3); wherein the V_{β} and the V_{α} are connected via a first peptide linker (L1);
   2) a heavy chain variable (V_{H}) comprising SEQ ID NO: 21 (CDR1) or SEQ ID NO: 32 (CDR1), SEQ ID NO: 25 (CDR2) or SEQ ID NO: 33 (CDR2), and SEQ ID NO: 29 (CDR3) or SEQ ID NO: 34 (CDR3); and
   3) a first heavy chain constant region (CH1CH2CH3) comprising the amino acid sequence selected from a group consisting of SEQ ID NO: 37, SEQ ID NO: 38 and SEQ ID NO: 39;
      wherein the V_{α} of the sTCR and the V_{H} are connected via a second peptide linker (L2), and
      wherein the first heavy chain region is in the form of V_{β}-L1-V_{α}-L2-V_{H}-CH1CH2CH3;
b) a second heavy chain region (CH2′CH3′) comprising the amino acid sequence of SEQ ID NO: 16; and
c) a light chain comprising:
   1) a light chain variable (V_{L}) comprising SEQ ID NO: 22 (CDR1) or SEQ ID NO: 81 (CDR1), SEQ ID NO: 26 (CDR2) or SEQ ID NO: 82 (CDR2), and SEQ ID NO: 30 (CDR3) or SEQ ID NO: 83 (CDR3); and
   2) a kappa constant light chain (C_{κ}) having the amino acid sequence of SEQ ID NO: 17;

wherein the CH2CH3 of the first heavy chain and CH2′CH3′ of the second heavy chain form a dimeric Fc region, to form a bispecific molecule with the following form:

V_{β}-L1-V_{α}-L2-V_{H}-CH1CH2CH3V_{L}-CκCH2′CH3′.

In one embodiment, the present invention provides a bispecific molecule which binds to both human CD3 and a complex of the peptide Survivin, wherein the bispecific molecule comprises:
a) a first heavy chain region comprising
   1) a single-chain soluble T cell receptor (sTCR) which binds to a complex of the peptide Survivin, wherein the complex comprises the amino acid sequence of SEQ ID NO: 40 and the HLA-A2 molecule, wherein the sTCR comprises:
      i. a sTCR variable beta region (V_{β}) comprising SEQ ID NO: 20 (CDR1), SEQ ID NO: 24 (CDR2), and SEQ ID NO: 28 (CDR3); and
      ii. a sTCR variable alpha region (V_{α}) comprising SEQ ID NO: 19 (CDR1), SEQ ID NO: 23 (CDR2), and SEQ ID NO: 27 (CDR3); wherein the V_{β} and the V_{α} are connected via a first peptide linker (L1) comprising the amino acid sequence of SEQ ID NO: 1;
   2) a heavy chain variable (V_{H}) comprising SEQ ID NO: 21 (CDR1), SEQ ID NO: 25 (CDR2), and SEQ ID NO: 29 (CDR3); and
   3) a first heavy chain constant region (CH1CH2CH3) comprising the amino acid sequence of SEQ ID NO: 37;
      wherein the V_{α} of the sTCR and the V_{H} are connected via a second peptide linker (L2), and
      wherein the first heavy chain region is in the form of V_{β}-L1-V_{α}-L2-V_{H}-CH1CH2CH3;
b) a second heavy chain region (CH2′CH3′) comprising the amino acid sequence of SEQ ID NO: 16; and
c) a light chain comprising:
   1) a light chain variable (V_{L}) comprising SEQ ID NO: 22 (CDR1), SEQ ID NO: 26 (CDR2), and SEQ ID NO: 30 (CDR3); and
   2) a kappa constant light chain (C_{κ}) having the amino acid sequence of SEQ ID NO: 17;

wherein the CH2CH3 of the first heavy chain and CH2′CH3′ of the second heavy chain form a dimeric Fc region, to form a bispecific molecule with the following form

V_{β}-L1-V_{α}-L2-V_{H}-CH1CH2CH3V_{L}-CκCH2′CH3′.

In one embodiment, the present invention provides a bispecific molecule which binds to both human CD3 and a complex of the peptide Survivin, wherein the bispecific molecule comprises:
a) a first heavy chain region comprising
   1) a single-chain soluble T cell receptor (sTCR) which binds to a complex of the peptide Survivin, wherein the complex comprises the amino acid sequence of SEQ ID NO: 40 and the HLA-A2 molecule, wherein the sTCR comprises:
      i. a sTCR variable beta region (V_{β}) comprising the amino acid sequence selected from a group consisting of SEQ ID NO: 2, SEQ ID NO: 3, SEQ ID NO: 4 and SEQ ID NO: 5; and
      ii. a sTCR variable alpha region (V_{α}) comprising the amino acid sequence selected from a group consisting of SEQ ID NO: 6, SEQ ID NO: 7 and SEQ ID NO: 8;
         wherein the V_{β} and the V_{α} are connected via a first peptide linker (L1);
   2) a heavy chain variable (V_{H}) comprising the amino acid sequence of SEQ ID NO: 9 or SEQ ID NO: 10; and
   3) a first heavy chain constant region (CH1CH2CH3) comprising the amino acid sequence selected from a group consisting of SEQ ID NO: 37, SEQ ID NO: 38 and SEQ ID NO: 39;
      wherein the V_{α} of the sTCR and the V_{H} are connected via a second peptide linker (L2), and
      wherein the first heavy chain region is in the form of V_{β}-L1-V_{α}-L2-V_{H}-CH1CH2CH3;
b) a second heavy chain region (CH2′CH3′) comprising the amino acid sequence of SEQ ID NO: 16; and
c) a light chain comprising:
   1) a light chain variable (V_{L}) comprising the amino acid sequence of SEQ ID NO: 11 or SEQ ID NO: 12; and
   2) a kappa constant light chain (C_{κ}) having the amino acid sequence of SEQ ID NO: 17;

wherein the CH2CH3 of the first heavy chain and CH2′CH3′ of the second heavy chain form a dimeric Fc region, to form a bispecific molecule with the following form:

V_{β}-L1-V_{α}-L2-V_{H}-CH1CH2CH3V_{L}-CκCH2′CH3′.

In one embodiment, the present invention provides a bispecific molecule which binds to both human CD3 and a complex of the peptide Survivin, wherein the bispecific molecule comprises:
a) a first heavy chain region comprising:
   1) a single-chain soluble T cell receptor (sTCR) which binds to a complex of the peptide Survivin, wherein the complex comprises the amino acid sequence of SEQ ID NO: 40 and the HLA-A2 molecule, wherein the sTCR comprises:
      i. a sTCR variable beta region (V_{β}) comprising the amino acid sequence of SEQ ID NO: 2; and
      ii. a sTCR variable alpha region (V_{α}) comprising the amino acid sequence of SEQ ID NO: 6;
         wherein the V_{β} and the V_{α} are connected via a first peptide linker (L1);
   2) a heavy chain variable (V_{H}) comprising the amino acid sequence of SEQ ID NO: 9; and
   3) a first heavy chain constant region (CH1CH2CH3) comprising the amino acid sequence of SEQ ID NO: 37;
      wherein the V_{α} of the sTCR and the V_{H} are connected via a second peptide linker (L2), and
      wherein the first heavy chain region is in the form of V_{β}-L1-V_{α}-L2-V_{H}-CH1CH2CH3;
b) a second heavy chain region (CH2′CH3′) comprising the amino acid sequence of SEQ ID NO: 16; and
c) a light chain comprising:
   1) a light chain variable (V_{L}) comprising the amino acid sequence of SEQ ID NO: 11; and
   2) a kappa constant light chain (C_{κ}) having the amino acid sequence of SEQ ID NO: 17;

wherein the CH2CH3 of the first heavy chain and CH2′CH3′ of the second heavy chain form a dimeric Fc region, to form a bispecific molecule with the following form:

V_{β}-L1-V_{α}-L2-V_{H}-CH1CH2CH3V_{L}-CκCH2′CH3′.

In one embodiment, the present invention provides a bispecific molecule which binds to human CD3 and a complex of the peptide Survivin, wherein the complex comprises the amino acid sequence of SEQ ID NO: 40 and the HLA-A2 molecule, wherein the bispecific molecule comprises:
a) a first heavy chain comprising the amino acid sequence of SEQ ID NO: 36,
b) a second heavy chain comprising the amino acid sequence of SEQ ID NO: 16 and
c) a light chain comprising the amino acid sequence of SEQ ID NO: 76.

In one embodiment, the present invention provides a bispecific molecule which binds to human CD3 and a complex of the peptide Survivin, wherein the complex comprises the amino acid sequence of SEQ ID NO: 40 and the HLA-A2 molecule, wherein the bispecific molecule comprises:
a) a heavy chain comprising the amino acid sequence of SEQ ID NO: 88,
b) a second heavy chain comprising the amino acid sequence of SEQ ID NO: 16 and
c) a light chain comprising the amino acid sequence of SEQ ID NO: 76.

In one embodiment, the present invention provides a bispecific molecule comprising:
a) a single-chain soluble T cell receptor (sTCR), which binds to a complex of the peptide Survivin, wherein the complex comprises the amino acid sequence of SEQ ID NO: 40 and the HLA-A2 molecule, wherein the sTCR comprises:
   (1) a sTCR variable beta region (V_{β}) comprising the amino acid sequence of SEQ ID NO: 5, and
   (2) a sTCR variable alpha region (V_{α}) comprising the amino acid sequence of SEQ ID NO: 6,
   wherein the V_{β} and V_{α} regions of the sTCR are connected via a first peptide linker (L1);
b) an antigen binding fragment (Fab) which binds to human CD3 (anti-CD3-Fab), wherein the anti-CD3-Fab comprises:
   (1) a heavy chain region comprising a heavy chain variable (V_{H}) comprising the amino acid sequence of SEQ ID NO: 9; and a heavy chain constant domain 1 (CH1) comprising the amino acid sequence of SEQ ID NO: 18, and
   (2) a light chain region comprising a light chain variable (V_{L}) comprising the amino acid sequence of SEQ ID NO: 11; and a kappa constant light chain (C_{κ}) comprising the amino acid sequence of SEQ ID NO: 17,
   wherein the V_{α} of the sTCR and the V_{H} of the anti-CD3-Fab are connected via a second peptide linker (L2); and
c) a fragment crystallizable region (Fc) comprising:
   (1) a first constant region comprising a first constant domain 2 (CH2) and a first constant domain 3 (CH3), wherein the first constant region (CH2CH3) comprises the amino acid sequence of SEQ ID NO: 13, and
   (2) a second constant region comprising a second constant domain 2 (CH2′) and a second constant domain 3 (CH3′), wherein the second constant region (CH2′CH3′) comprises the amino acid sequence of SEQ ID NO: 16,

wherein the bispecific molecule is in the form of

V_{β}-L1-V_{α}-L2-V_{H}-CH1CH2CH3V_{L}-CκCH2′CH3′.

In another aspect, the present invention provides a bispecific molecule which binds to both human CD3 and a complex of the peptide Survivin, wherein the bispecific molecule comprises:
a) a first heavy chain region comprising
   1) a single-chain soluble T cell receptor (sTCR) which binds to a complex of the peptide Survivin, wherein the complex comprises the amino acid sequence of SEQ ID NO: 40 and the HLA-A2 molecule, wherein the sTCR comprises:
      i. a sTCR variable beta region (V_{β}) comprising the amino acid sequence of SEQ ID NO: 5; and
      ii. a sTCR variable alpha region (V_{α}) comprising the amino acid sequence of SEQ ID NO: 6;
         wherein the V_{β} and the V_{α} are connected via a first peptide linker (L1);
   2) a heavy chain variable (V_{H}) comprising the amino acid sequence of SEQ ID NO: 9; and
   3) a first heavy chain constant region (CH1CH2CH3) comprising the amino acid sequence of SEQ ID NO: 37;
      wherein the V_{α} of the sTCR and the V_{H} are connected via a second peptide linker (L2), and
      wherein the first heavy chain region is in the form of V_{β}-L1-V_{α}-L2-V_{H}-CH1CH2CH3;
b) a second heavy chain region (CH2′CH3′) comprising the amino acid sequence of SEQ ID NO: 16; and
c) a light chain comprising:
   1) a light chain variable (V_{L}) comprising the amino acid sequence of SEQ ID NO: 11; and
   2) a kappa constant light chain (C_{κ}) having the amino acid sequence of SEQ ID NO: 17;

wherein the CH2CH3 of the first heavy chain and CH2′CH3′ of the second heavy chain form a dimeric Fc region, to form a bispecific molecule with the following form:

V_{β}-L1-V_{α}-L2-V_{H}-CH1CH2CH3V_{L}-CκCH2′CH3′.

In one embodiment, the present invention provides a bispecific molecule comprising:
a) a single-chain soluble T cell receptor (sTCR), which binds to a complex of the peptide Survivin, wherein the complex comprises the amino acid sequence of SEQ ID NO: 40 and the HLA-A2 molecule, wherein the sTCR comprises:
   (1) a sTCR variable beta region (V_{β}) comprising the amino acid sequence of SEQ ID NO: 3, and
   (2) a sTCR variable alpha region (V_{α}) comprising the amino acid sequence of SEQ ID NO: 7,
      wherein the V_{β} and V_{α} regions of the sTCR are connected via a first peptide linker (L1);
b) an antigen binding fragment (Fab) which binds to human CD3 (anti-CD3-Fab), wherein the anti-CD3-Fab comprises:
   (1) a heavy chain region comprising a heavy chain variable (V_{H}) comprising the amino acid sequence of SEQ ID NO: 9; and a heavy chain constant domain 1 (CH1) comprising the amino acid sequence of SEQ ID NO: 35, and
   (2) a light chain region comprising a light chain variable (V_{L}) comprising the amino acid sequence of SEQ ID NO: 11; and a kappa constant light chain (C_{κ}) comprising the amino acid sequence of SEQ ID NO: 17,
      wherein the V_{α} of the sTCR and the V_{H} of the anti-CD3-Fab are connected via a second peptide linker (L2); and
c) a fragment crystallizable region (Fc) comprising:
   (1) a constant region comprising a constant domain 2 (CH2) and a constant domain 3 (CH3), wherein the constant region (CH2CH3) comprises the amino acid sequence of SEQ ID NO: 14,

wherein the bispecific molecule is in the form of

V_{β}-L1-V_{α}-L2-V_{H}-CH1CH2CH3V_{L}-Cκ.

In another aspect, the present invention provides a bispecific molecule which binds to both human CD3 and a complex of the peptide Survivin, wherein the bispecific molecule comprises:
a) a heavy chain region comprising
   1) a single-chain soluble T cell receptor (sTCR) which binds to a complex of the peptide Survivin, wherein the complex comprises the amino acid sequence of SEQ ID NO: 40 and the HLA-A2 molecule, wherein the sTCR comprises:
      i. a sTCR variable beta region (V_{β}) comprising the amino acid sequence of SEQ ID NO: 3; and
      ii. a sTCR variable alpha region (V_{α}) comprising the amino acid sequence of SEQ ID NO: 7;
         wherein the V_{β} and the V_{α} are connected via a first peptide linker (L1);
   2) a heavy chain variable (V_{H}) comprising the amino acid sequence of SEQ ID NO: 9; and
   3) a heavy chain constant region (CH1CH2CH3) comprising the amino acid sequence of SEQ ID NO: 38;
      wherein the V_{α} of the sTCR and the V_{H} are connected via a second peptide linker (L2), and
      wherein the heavy chain region is in the form of V_{β}-L1-V_{α}-L2-V_{H}-CH1CH2CH3; and
b) a light chain comprising:
   1) a light chain variable (V_{L}) comprising the amino acid sequence of SEQ ID NO: 11; and
   2) a kappa constant light chain (C_{κ}) having the amino acid sequence of SEQ ID NO: 17;

to form a bispecific molecule with the following form:

V_{β}-L1-V_{α}-L2-V_{H}-CH1CH2CH3V_{L}-Cκ.

In one embodiment, the present invention provides a bispecific molecule comprising:
a) a single-chain soluble T cell receptor (sTCR), which binds to a complex of the peptide Survivin, wherein the complex comprises the amino acid sequence of SEQ ID NO: 40 and the HLA-A2 molecule, wherein the sTCR comprises:
   (1) a sTCR variable alpha region (V_{α}) comprising the amino acid sequence of SEQ ID NO: 7, and
   (2) a sTCR variable beta region (V_{β}) comprising the amino acid sequence of SEQ ID NO: 3,
      wherein the V_{β} and V_{α} regions of the sTCR are connected via a first peptide linker (L1);
b) an antigen binding fragment (Fab) which binds to human CD3 (anti-CD3-Fab), wherein the anti-CD3-Fab comprises:
   (1) a heavy chain region comprising a heavy chain variable (V_{H}) comprising the amino acid sequence of SEQ ID NO: 9; and a heavy chain constant domain 1 (CH1) comprising the amino acid sequence of SEQ ID NO: 35, and
   (2) a light chain region comprising a light chain variable (V_{L}) comprising the amino acid sequence of SEQ ID NO: 11; and a kappa constant light chain (C_{κ}) comprising the amino acid sequence of SEQ ID NO: 17,
      wherein the V_{β} of the sTCR and the V_{H} of the anti-CD3-Fab are connected via a second peptide linker (L2); and
c) a fragment crystallizable region (Fc) comprising:
   (1) a constant region comprising a constant domain 2 (CH2) and a constant domain 3 (CH3), wherein the constant region (CH2CH3) comprises the amino acid sequence of SEQ ID NO: 14,

wherein the bispecific molecule is in the form of

V_{β}-L1-V_{α}-L2-V_{H}-CH1CH2CH3V_{L}-Cκ.

In another aspect, the present invention provides a bispecific molecule which binds to both human CD3 and a complex of the peptide Survivin, wherein the bispecific molecule comprises:
a) a heavy chain region comprising
   1) a single-chain soluble T cell receptor (sTCR) which binds to a complex of the peptide Survivin, wherein the complex comprises the amino acid sequence of SEQ ID NO: 40 and the HLA-A2 molecule, wherein the sTCR comprises:
      i. a sTCR variable alpha region (V_{α}) comprising the amino acid sequence of SEQ ID NO: 7; and
      ii. a sTCR variable beta region (V_{β}) comprising the amino acid sequence of SEQ ID NO: 3;
      wherein the V_{β} and the V_{α} are connected via a first peptide linker (L1);
   2) a heavy chain variable (V_{H}) comprising the amino acid sequence of SEQ ID NO: 9; and
   3) a heavy chain constant region (CH1CH2CH3) comprising the amino acid sequence of SEQ ID NO: 38;
      wherein the V_{β} of the sTCR and the V_{H} are connected via a second peptide linker (L2), and
      wherein the heavy chain region is in the form of V_{α}-L1-V_{β}-L2-V_{H}-CH1CH2CH3; and
b) a light chain comprising:
   1) a light chain variable (V_{L}) comprising the amino acid sequence of SEQ ID NO: 11; and
   2) a kappa constant light chain (C_{κ}) having the amino acid sequence of SEQ ID NO: 17;

to form a bispecific molecule with the following form:

V_{β}-L1-V_{α}-L2-V_{H}-CH1CH2CH3V_{L}-Cκ.

In one embodiment, the present invention provides a bispecific molecule comprising:
a) a single-chain soluble T cell receptor (sTCR), which binds to a complex of the peptide Survivin, wherein the complex comprises the amino acid sequence of SEQ ID NO: 40 and the HLA-A2 molecule, wherein the sTCR comprises:
   (1) a sTCR variable alpha region (V_{α}) comprising the amino acid sequence of SEQ ID NO: 7, and
   (2) a sTCR variable beta region (V_{β}) comprising the amino acid sequence of SEQ ID NO: 3,
      wherein the V_{β} and V_{α} regions of the sTCR are connected via a first peptide linker (L1);
b) an antigen binding fragment (Fab) which binds to human CD3 (anti-CD3-Fab), wherein the anti-CD3-Fab comprises:
   (1) a heavy chain region comprising a heavy chain variable (V_{H}) comprising the amino acid sequence of SEQ ID NO: 9; and a heavy chain constant domain 1 (CH1) comprising the amino acid sequence of SEQ ID NO: 18, and
   (2) a light chain region comprising a light chain variable (V_{L}) comprising the amino acid sequence of SEQ ID NO: 11; and a kappa constant light chain (C_{κ}) comprising the amino acid sequence of SEQ ID NO: 17, and
      wherein the V_{β} of the sTCR and the V_{H} of the anti-CD3-Fab are connected via a second peptide linker (L2);

wherein the bispecific molecule is in the form of

V_{β}-L1-V_{α}-L2-V_{H}-CH1CH2CH3V_{L}-Cκ.

In another aspect, the present invention provides a bispecific molecule which binds to both human CD3 and a complex of the peptide Survivin, wherein the bispecific molecule comprises:
a) a heavy chain region comprising
   1) a single-chain soluble T cell receptor (sTCR) which binds to a complex of the peptide Survivin, wherein the complex comprises the amino acid sequence of SEQ ID NO: 40 and the HLA-A2 molecule, wherein the sTCR comprises:
      i. a sTCR variable alpha region (V_{α}) comprising the amino acid sequence of SEQ ID NO: 7; and
      ii. a sTCR variable beta region (V_{β}) comprising the amino acid sequence of SEQ ID NO: 3;
         wherein the V_{β} and the V_{α} are connected via a first peptide linker (L1);
   2) a heavy chain variable (V_{H}) comprising the amino acid sequence of SEQ ID NO: 9; and
   3) a heavy chain constant region (CH1) comprising the amino acid sequence of SEQ ID NO: 18;
      wherein the V_{β} of the sTCR and the V_{H} are connected via a second peptide linker (L2), and
      wherein the heavy chain region is in the form of V_{α}-L1-V_{β}-L2-V_{H}—CH1; and
b) a light chain comprising:
   1) a light chain variable (V_{L}) comprising the amino acid sequence of SEQ ID NO: 11; and
   2) a kappa constant light chain (C_{κ}) having the amino acid sequence of SEQ ID NO: 17;

to form a bispecific molecule with the following form:

V_{β}-L1-V_{α}-L2-V_{H}-CH1CH2CH3V_{L}-Cκ.

In one embodiment, the present invention provides a bispecific molecule comprising:
a) a single-chain soluble T cell receptor (sTCR), which binds to a complex of the peptide Survivin, wherein the complex comprises the amino acid sequence of SEQ ID NO: 40 and the HLA-A2 molecule, wherein the sTCR comprises:
   (1) a sTCR variable beta region (V_{β}) comprising the amino acid sequence of SEQ ID NO: 3, and
   (2) a sTCR variable alpha region (V_{α}) comprising the amino acid sequence of SEQ ID NO: 7,
      wherein the V_{β} and V_{α} regions of the sTCR are connected via a first peptide linker (L1);
b) an antigen binding fragment (Fab) which binds to human CD3 (anti-CD3-Fab), wherein the anti-CD3-Fab comprises:
   (1) a heavy chain region comprising a heavy chain variable (V_{H}) comprising the amino acid sequence of SEQ ID NO: 9; and a heavy chain constant domain 1 (CH1) comprising the amino acid sequence of SEQ ID NO: 18, and
   (2) a light chain region comprising a light chain variable (V_{L}) comprising the amino acid sequence of SEQ ID NO: 11; and a kappa constant light chain (C_{κ}) comprising the amino acid sequence of SEQ ID NO: 17, and
      wherein the V_{α} of the sTCR and the V_{H} of the anti-CD3-Fab are connected via a second peptide linker (L2);

wherein the bispecific molecule is in the form of

V_{β}-L1-V_{α}-L2-V_{H}-CH1CH2CH3V_{L}-Cκ.

In another aspect, the present invention provides a bispecific molecule which binds to both human CD3 and a complex of the peptide Survivin, wherein the bispecific molecule comprises:
a) a heavy chain region comprising
   1) a single-chain soluble T cell receptor (sTCR) which binds to a complex of the peptide Survivin, wherein the complex comprises the amino acid sequence of SEQ ID NO: 40 and the HLA-A2 molecule, wherein the sTCR comprises:
      i. a sTCR variable beta region (V_{β}) comprising the amino acid sequence of SEQ ID NO: 3; and
      ii. a sTCR variable alpha region (V_{α}) comprising the amino acid sequence of SEQ ID NO: 7;
         wherein the V_{β} and the V_{α} are connected via a first peptide linker (L1);
   2) a heavy chain variable (V_{H}) comprising the amino acid sequence of SEQ ID NO: 9; and
   3) a heavy chain constant region (CH1) comprising the amino acid sequence of SEQ ID NO: 18;
      wherein the V_{α} of the sTCR and the V_{H} are connected via a second peptide linker (L2), and
      wherein the heavy chain region is in the form of V_{β}-L1-V_{α}-L2-V_{H}—CH1; and
b) a light chain comprising:
   1) a light chain variable (V_{L}) comprising the amino acid sequence of SEQ ID NO: 11; and
   2) a kappa constant light chain (C_{κ}) having the amino acid sequence of SEQ ID NO: 17;
to form a bispecific molecule with the following form:

V_{β}-L1-V_{α}-L2-V_{H}-CH1CH2CH3V_{L}-Cκ.

In one embodiment, the present invention provides a bispecific molecule comprising:
a) a single-chain soluble T cell receptor (sTCR), which binds to a complex of the peptide Survivin, wherein the complex comprises the amino acid sequence of SEQ ID NO: 40 and the HLA-A2 molecule, wherein the sTCR comprises:
   (1) a sTCR variable beta region (V_{β}) comprising the amino acid sequence of SEQ ID NO: 4, and
   (2) a sTCR variable alpha region (V_{α}) comprising the amino acid sequence of SEQ ID NO: 8,
      wherein the V_{β} and V_{α} regions of the sTCR are connected via a first peptide linker (L1);
b) an antigen binding fragment (Fab) which binds to human CD3 (anti-CD3-Fab), wherein the anti-CD3-Fab comprises:
   (1) a heavy chain region comprising a heavy chain variable (V_{H}) comprising the amino acid sequence of SEQ ID NO: 9; and a heavy chain constant domain 1 (CH1) comprising the amino acid sequence of SEQ ID NO: 35, and
   (2) a light chain region comprising a light chain variable (V_{L}) comprising the amino acid sequence of SEQ ID NO: 11; and a kappa constant light chain (C_{κ}) comprising the amino acid sequence of SEQ ID NO: 17,
      wherein the V_{α} of the sTCR and the V_{H} of the anti-CD3-Fab are connected via a second peptide linker (L2); and
c) a fragment crystallizable region (Fc) comprising:
   (1) a constant region comprising a constant domain 2 (CH2) and a constant domain 3 (CH3), wherein the constant region (CH2CH3) comprises the amino acid sequence of SEQ ID NO: 14,

wherein the bispecific molecule is in the form of

V_{β}-L1-V_{α}-L2-V_{H}-CH1CH2CH3V_{L}-Cκ.

In another aspect, the present invention provides a bispecific molecule which binds to both human CD3 and a complex of the peptide Survivin, wherein the bispecific molecule comprises:
a) a heavy chain region comprising
   1) a single-chain soluble T cell receptor (sTCR) which binds to a complex of the peptide Survivin, wherein the complex comprises the amino acid sequence of SEQ ID NO: 40 and the HLA-A2 molecule, wherein the sTCR comprises:
      i. a sTCR variable beta region (V_{β}) comprising the amino acid sequence of SEQ ID NO: 4; and
      ii. a sTCR variable alpha region (V_{α}) comprising the amino acid sequence of SEQ ID NO: 8;
         wherein the V_{β} and the V_{α} are connected via a first peptide linker (L1);
   2) a heavy chain variable (V_{H}) comprising the amino acid sequence of SEQ ID NO: 9; and
   3) a heavy chain constant region (CH1CH2CH3) comprising the amino acid sequence of SEQ ID NO: 38;
      wherein the V_{α} of the sTCR and the V_{H} are connected via a second peptide linker (L2), and
      wherein the heavy chain region is in the form of V_{β}-L1-V_{α}-L2-V_{H}-CH1CH2CH3; and
b) a light chain comprising:
   1) a light chain variable (V_{L}) comprising the amino acid sequence of SEQ ID NO: 11; and
   2) a kappa constant light chain (C_{κ}) having the amino acid sequence of SEQ ID NO: 17;

to form a bispecific molecule with the following form:

V_{β}-L1-V_{α}-L2-V_{H}-CH1CH2CH3V_{L}-Cκ.

In one embodiment, the present invention provides a bispecific molecule comprising:
a) a single-chain soluble T cell receptor (sTCR), which binds to a complex of the peptide Survivin, wherein the complex comprises the amino acid sequence of SEQ ID NO: 40 and the HLA-A2 molecule, wherein the sTCR comprises:
   (1) a sTCR variable beta region (V_{β}) comprising the amino acid sequence of SEQ ID NO: 5, and
   (2) a sTCR variable alpha region (V_{α}) comprising the amino acid sequence of SEQ ID NO: 6,
      wherein the V_{β} and V_{α} regions of the sTCR are connected via a first peptide linker (L1);
b) an antigen binding fragment (Fab) which binds to human CD3 (anti-CD3-Fab), wherein the anti-CD3-Fab comprises:
   (1) a heavy chain region comprising a heavy chain variable (V_{H}) comprising the amino acid sequence of SEQ ID NO: 9; and a heavy chain constant domain 1 (CH1) comprising the amino acid sequence of SEQ ID NO: 35, and
   (2) a light chain region comprising a light chain variable (V_{L}) comprising the amino acid sequence of SEQ ID NO: 11; and a kappa constant light chain (C_{κ}) comprising the amino acid sequence of SEQ ID NO: 17,
      wherein the V_{α} of the sTCR and the V_{H} of the anti-CD3-Fab are connected via a second peptide linker (L2); and
c) a fragment crystallizable region (Fc) comprising:
   (1) a constant region comprising a constant domain 2 (CH2) and a constant domain 3 (CH3), wherein the constant region (CH2CH3) comprises the amino acid sequence of SEQ ID NO: 14,

wherein the bispecific molecule is in the form of

V_{β}-L1-V_{α}-L2-V_{H}-CH1CH2CH3V_{L}-Cκ.

In another aspect, the present invention provides a bispecific molecule which binds to both human CD3 and a complex of the peptide Survivin, wherein the bispecific molecule comprises:
a) a heavy chain region comprising
   1) a single-chain soluble T cell receptor (sTCR) which binds to a complex of the peptide Survivin, wherein the complex comprises the amino acid sequence of SEQ ID NO: 40 and the HLA-A2 molecule, wherein the sTCR comprises:
      i. a sTCR variable beta region (V_{β}) comprising the amino acid sequence of SEQ ID NO: 5; and
      ii. a sTCR variable alpha region (V_{α}) comprising the amino acid sequence of SEQ ID NO: 6;
         wherein the V_{β} and the V_{α} are connected via a first peptide linker (L1);
   2) a heavy chain variable (V_{H}) comprising the amino acid sequence of SEQ ID NO: 9; and
   3) a heavy chain constant region (CH1CH2CH3) comprising the amino acid sequence of SEQ ID NO: 38;
      wherein the V_{α} of the sTCR and the V_{H} are connected via a second peptide linker (L2), and
      wherein the heavy chain region is in the form of V_{β}-L1-V_{α}-L2-V_{H}-CH1CH2CH3; and
b) a light chain comprising:
   1) a light chain variable (V_{L}) comprising the amino acid sequence of SEQ ID NO: 11; and
   2) a kappa constant light chain (C_{κ}) having the amino acid sequence of SEQ ID NO: 17;

to form a bispecific molecule with the following form:

V_{β}-L1-V_{α}-L2-V_{H}-CH1CH2CH3V_{L}-Cκ.

In one embodiment, the present invention provides a bispecific molecule comprising:
a) a single-chain soluble T cell receptor (sTCR), which binds to a complex of the peptide Survivin, wherein the complex comprises the amino acid sequence of SEQ ID NO: 40 and the HLA-A2 molecule, wherein the sTCR comprises:
   (1) a sTCR variable beta region (V_{β}) comprising the amino acid sequence of SEQ ID NO: 5, and
   (2) a sTCR variable alpha region (V_{α}) comprising the amino acid sequence of SEQ ID NO: 6,
      wherein the V_{β} and V_{α} regions of the sTCR are connected via a first peptide linker (L1);
b) an antigen binding fragment (Fab) which binds to human CD3 (anti-CD3-Fab), wherein the anti-CD3-Fab comprises:
   (1) a heavy chain region comprising a heavy chain variable (V_{H}) comprising the amino acid sequence of SEQ ID NO: 10; and a heavy chain constant domain 1 (CH1) comprising the amino acid sequence of SEQ ID NO: 35, and
   (2) a light chain region comprising a light chain variable (V_{L}) comprising the amino acid sequence of SEQ ID NO: 12; and a kappa constant light chain (C_{κ}) comprising the amino acid sequence of SEQ ID NO: 17,
      wherein the V_{α} of the sTCR and the V_{H} of the anti-CD3-Fab are connected via a second peptide linker (L2); and
c) a fragment crystallizable region (Fc) comprising:
   (2) a constant region comprising at constant domain 2 (CH2) and a constant domain 3 (CH3), wherein the constant region (CH2CH3) comprises the amino acid sequence of SEQ ID NO: 14,

wherein the bispecific molecule is in the form of

V_{β}-L1-V_{α}-L2-V_{H}-CH1CH2CH3V_{L}-Cκ.

In another aspect, the present invention provides a bispecific molecule which binds to both human CD3 and a complex of the peptide Survivin, wherein the bispecific molecule comprises:
a) a heavy chain region comprising
   1) a single-chain soluble T cell receptor (sTCR) which binds to a complex of the peptide Survivin, wherein the complex comprises the amino acid sequence of SEQ ID NO: 40 and the HLA-A2 molecule, wherein the sTCR comprises:
      i. a sTCR variable beta region (V_{β}) comprising the amino acid sequence of SEQ ID NO: 5; and
      ii. a sTCR variable alpha region (V_{α}) comprising the amino acid sequence of SEQ ID NO: 6;
         wherein the V_{β} and the V_{α} are connected via a first peptide linker (L1);
   2) a heavy chain variable (V_{H}) comprising the amino acid sequence of SEQ ID NO: 10; and
   3) a heavy chain constant region (CH1CH2CH3) comprising the amino acid sequence of SEQ ID NO: 38;
      wherein the V_{α} of the sTCR and the V_{H} are connected via a second peptide linker (L2), and
      wherein the heavy chain region is in the form of V_{β}-L1-V_{α}-L2-V_{H}-CH1CH2CH3; and
b) a light chain comprising:
   1) a light chain variable (V_{L}) comprising the amino acid sequence of SEQ ID NO: 12; and
   2) a kappa constant light chain (C_{κ}) having the amino acid sequence of SEQ ID NO: 17;

to form a bispecific molecule with the following form:

V_{β}-L1-V_{α}-L2-V_{H}-CH1CH2CH3V_{L}-Cκ.

In one embodiment, the present invention provides a bispecific molecule comprising:
a) a single-chain soluble T cell receptor (sTCR), which binds to a complex of the peptide Survivin, wherein the complex comprises the amino acid sequence of SEQ ID NO: 40 and the HLA-A2 molecule, wherein the sTCR comprises:
   (1) a sTCR variable beta region (V_{β}) comprising the amino acid sequence of SEQ ID NO: 5, and
   (2) a sTCR variable alpha region (V_{α}) comprising the amino acid sequence of SEQ ID NO: 6,
      wherein the V_{β} and V_{α} regions of the sTCR are connected via a first peptide linker (L1);
b) an antigen binding fragment (Fab) which binds to human CD3 (anti-CD3-Fab), wherein the anti-CD3-Fab comprises:
   (1) a heavy chain region comprising a heavy chain variable (V_{H}) comprising the amino acid sequence of SEQ ID NO: 9; and a heavy chain constant domain 1 (CH1) comprising the amino acid sequence of SEQ ID NO: 18, and
   (2) a light chain region comprising a light chain variable (V_{L}) comprising the amino acid sequence of SEQ ID NO: 11; and a kappa constant light chain (C_{κ}) comprising the amino acid sequence of SEQ ID NO: 17, and
      wherein the V_{α} of the sTCR and the V_{H} of the anti-CD3-Fab are connected via a second peptide linker (L2);

wherein the bispecific molecule is in the form of

V_{β}-L1-V_{α}-L2-V_{H}-CH1CH2CH3V_{L}-Cκ.

In another aspect, the present invention provides a bispecific molecule which binds to both human CD3 and a complex of the peptide Survivin, wherein the bispecific molecule comprises:
a) a heavy chain region comprising
   1) a single-chain soluble T cell receptor (sTCR) which binds to a complex of the peptide Survivin, wherein the complex comprises the amino acid sequence of SEQ ID NO: 40 and the HLA-A2 molecule, wherein the sTCR comprises:
      i. a sTCR variable beta region (V_{β}) comprising the amino acid sequence of SEQ ID NO: 5; and
      ii. a sTCR variable alpha region (V_{α}) comprising the amino acid sequence of SEQ ID NO: 6;
         wherein the V_{β} and the V_{α} are connected via a first peptide linker (L1);
   2) a heavy chain variable (V_{H}) comprising the amino acid sequence of SEQ ID NO: 9; and
   3) a heavy chain constant region (CH1) comprising the amino acid sequence of SEQ ID NO: 18;
      wherein the V_{α} of the sTCR and the V_{H} are connected via a second peptide linker (L2), and
      wherein the heavy chain region is in the form of V_{β}-L1-V_{α}-L2-V_{H}—CH1; and
b) a light chain comprising:
   1) a light chain variable (V_{L}) comprising the amino acid sequence of SEQ ID NO: 11; and
   2) a kappa constant light chain (C_{κ}) having the amino acid sequence of SEQ ID NO: 17;

to form a bispecific molecule with the following form:

V_{β}-L1-V_{α}-L2-V_{H}-CH1CH2CH3V_{L}-Cκ.

In one embodiment, the present invention provides a bispecific molecule comprising:
a) a single-chain soluble T cell receptor (sTCR), which binds to a complex of the peptide Survivin, wherein the complex comprises the amino acid sequence of SEQ ID NO: 40 and the HLA-A2 molecule, wherein the sTCR comprises:
   (1) a sTCR variable beta region (V_{β}) comprising the amino acid sequence of SEQ ID NO: 5, and
   (2) a sTCR variable alpha region (V_{α}) comprising the amino acid sequence of SEQ ID NO: 6,
      wherein the V_{β} and V_{α} regions of the sTCR are connected via a first peptide linker (L1);
b) an antigen binding fragment (Fab) which binds to human CD3 (anti-CD3-Fab), wherein the anti-CD3-Fab comprises:
   (1) a heavy chain region comprising a heavy chain variable (V_{H}) comprising the amino acid sequence of SEQ ID NO: 10; and a heavy chain constant domain 1 (CH1) comprising the amino acid sequence of SEQ ID NO: 18, and
   (2) a light chain region comprising a light chain variable (V_{L}) comprising the amino acid sequence of SEQ ID NO: 12; and a kappa constant light chain (C_{κ}) comprising the amino acid sequence of SEQ ID NO: 17, and
      wherein the V_{α} of the sTCR and the V_{H} of the anti-CD3-Fab are connected via a second peptide linker (L2);
wherein the bispecific molecule is in the form of

V_{β}-L1-V_{α}-L2-V_{H}-CH1CH2CH3V_{L}-Cκ.

In another aspect, the present invention provides a bispecific molecule which binds to both human CD3 and a complex of the peptide Survivin, wherein the bispecific molecule comprises:
a) a heavy chain region comprising
   1) a single-chain soluble T cell receptor (sTCR) which binds to a complex of the peptide Survivin, wherein the complex comprises the amino acid sequence of SEQ ID NO: 40 and the HLA-A2 molecule, wherein the sTCR comprises:
      i. a sTCR variable beta region (V_{β}) comprising the amino acid sequence of SEQ ID NO: 5; and
      ii. a sTCR variable alpha region (V_{α}) comprising the amino acid sequence of SEQ ID NO: 6;
         wherein the V_{β} and the V_{α} are connected via a first peptide linker (L1);
   2) a heavy chain variable (V_{H}) comprising the amino acid sequence of SEQ ID NO: 10; and
   3) a heavy chain constant region (CH1) comprising the amino acid sequence of SEQ ID NO: 18;
      wherein the V_{α} of the sTCR and the V_{H} are connected via a second peptide linker (L2), and
      wherein the heavy chain region is in the form of V_{β}-L1-V_{α}-L2-V_{H}—CH1; and
b) a light chain comprising:
   1) a light chain variable (V_{L}) comprising the amino acid sequence of SEQ ID NO: 12; and
   2) a kappa constant light chain (C_{κ}) having the amino acid sequence of SEQ ID NO: 17;

to form a bispecific molecule with the following form:

V_{β}-L1-V_{α}-L2-V_{H}-CH1CH2CH3V_{L}-Cκ.

In one embodiment, the present invention provides a bispecific molecule comprising:
a) a single-chain soluble T cell receptor (sTCR), which binds to a complex of the peptide Survivin, wherein the complex comprises the amino acid sequence of SEQ ID NO: 40 and the HLA-A2 molecule, wherein the sTCR comprises:
   (1) a sTCR variable beta region (V_{β}) comprising the amino acid sequence of SEQ ID NO: 5, and
   (2) a sTCR variable alpha region (V_{α}) comprising the amino acid sequence of SEQ ID NO: 6,
      wherein the V_{β} and V_{α} regions of the sTCR are connected via a first peptide linker (L1);
b) an antigen binding fragment (Fab) which binds to human CD3 (anti-CD3-Fab), wherein the anti-CD3-Fab comprises:
   (1) a heavy chain region comprising a heavy chain variable (V_{H}) comprising the amino acid sequence of SEQ ID NO: 9; and a heavy chain constant domain 1 (CH1) comprising the amino acid sequence of SEQ ID NO: 35, and
   (2) a light chain region comprising a light chain variable (V_{L}) comprising the amino acid sequence of SEQ ID NO: 11; and a kappa constant light chain (C_{κ}) comprising the amino acid sequence of SEQ ID NO: 17,
      wherein the V_{α} of the sTCR and the V_{H} of the anti-CD3-Fab are connected via a second peptide linker (L2); and
c) a fragment crystallizable region (Fc) comprising:
   a constant region comprising at constant domain 2 (CH2) and a constant domain 3 (CH3), wherein the constant region (CH2CH3) comprises the amino acid sequence of SEQ ID NO: 15,

wherein the bispecific molecule is in the form of

V_{β}-L1-V_{α}-L2-V_{H}-CH1CH2CH3V_{L}-Cκ.

In another aspect, the present invention provides a bispecific molecule which binds to both human CD3 and a complex of the peptide Survivin, wherein the bispecific molecule comprises:
a) a heavy chain region comprising
   1) a single-chain soluble T cell receptor (sTCR) which binds to a complex of the peptide Survivin, wherein the complex comprises the amino acid sequence of SEQ ID NO: 40 and the HLA-A2 molecule, wherein the sTCR comprises:
      i. a sTCR variable beta region (V_{β}) comprising the amino acid sequence of SEQ ID NO: 5; and
      ii. a sTCR variable alpha region (V_{α}) comprising the amino acid sequence of SEQ ID NO: 6;
         wherein the V_{β} and the V_{α} are connected via a first peptide linker (L1);
   2) a heavy chain variable (V_{H}) comprising the amino acid sequence of SEQ ID NO: 9; and
   3) a heavy chain constant region (CH1CH2CH3) comprising the amino acid sequence of SEQ ID NO: 39;
      wherein the V_{α} of the sTCR and the V_{H} are connected via a second peptide linker (L2), and
      wherein the heavy chain region is in the form of V_{β}-L1-V_{α}-L2-V_{H}-CH1CH2CH3; and
b) a light chain comprising:
   1) a light chain variable (V_{L}) comprising the amino acid sequence of SEQ ID NO: 11; and
   2) a kappa constant light chain (C_{κ}) having the amino acid sequence of SEQ ID NO: 17;

to form a bispecific molecule with the following form:

V_{β}-L1-V_{α}-L2-V_{H}-CH1CH2CH3V_{L}-Cκ.

In one embodiment, the sTCR of the bispecific molecule binds to a peptide derived from human Survivin.

In one embodiment, the sTCR of the bispecific molecule binds to a peptide derived from human Survivin in complex with HLA-A2.

In one embodiment, the sTCR of the bispecific molecule binds to a peptide comprising the amino acid sequence of SEQ ID NO: 40.

In one embodiment, the sTCR of the bispecific molecule binds to a peptide comprising the amino acid sequence of SEQ ID NO: 40, which is derived from human Survivin in complex with HLA-A2.

In one embodiment, the V_{β} and V_{α} regions of the sTCR are connected via a first peptide linker (L1) comprising the amino acid sequence of SEQ ID NO: 1.

In one embodiment, the V_{α} and V_{β} regions of the sTCR are connected via a first peptide linker (L1) comprising the amino acid sequence of SEQ ID NO: 1.

In one embodiment, the V_{α} of the sTCR and V_{H} of the anti-CD3-Fab are connected via a second peptide linker comprising the amino acid sequence of SEQ ID NO: 1.

In one embodiment, the V_{β} of the sTCR and V_{H} of the anti-CD3-Fab are connected via a second peptide linker comprising the amino acid sequence of SEQ ID NO: 1.

As described in the examples below, several unexpected aspects of the molecules of the present invention have been identified. For example, the molecules of the present invention have a high affinity to Survivin as well as to human CD3. The Survivin TCR part of the molecules exhibit an apparent affinity of about 2 nM to Survivin, particularly remarkable high specificity directed towards a Survivin-derived peptide (SEQ ID NO: 40) complexed to HLA-A2, at the same time the molecules inhibit tumor growth and induce T cell activation and proliferation. Further, the molecules with KiH have a serum half-life of about 5 days, which is a significant improvement compared to the 0.5 hour half-life of the molecules that do not contain KiH.

In another aspect, the present disclosure pertains to a pharmaceutical composition comprising a bispecific molecule of the present invention.

In another aspect, the present disclosure pertains to a method of treating acute myeloid leukemia or B-cell non-Hodgkin's lymphoma, comprising administering to a patient in need thereof, a bispecific molecule of the present invention, or a pharmaceutical composition thereof.

In another aspect, the present disclosure pertains to nucleic acid molecules encoding the bispecific molecules of the present invention,

In another aspect, the present disclosure pertains to vectors comprising nucleic acid molecules encoding the bispecific molecules of the present invention.

In another aspect, the present disclosure pertains to host cells capable of producing the bispecific molecules of the present invention.

### EXAMPLES

The following Examples are provided for purposes of illustration, and not limitation.

### Example 1: TCR-CD3 Bispecific Molecule Generation

Bispecific molecules were generated. The polypeptide sequence of each component of the bispecific molecules is listed in Table 1, and the DNA sequence encoding such polypeptide is identified. CDRs within such polypeptides are underlined and their sequences are separately identified.

In some embodiments, the polypeptide sequence of CH2CH3, CH2′CH3′, CH1CH2CH3, Heavy Chain 1 and/or Heavy Chain 2 components of the bispecific molecules listed in Table 1 lack the C-terminal lysine, resulting in a C-terminal glycine residue.

In some embodiments, the polypeptide sequence of CH1 component of V_{α}V_{β}-FTab, V_{β} V_{α}-FTab-1, V_{β} V_{α}-FTab-2, or V_{β} V_{α}-FTab-3 listed in Table 1 further includes a 6-His tag (HHHHHH, SEQ ID NO: 90) placed at the C-terminus of the CH1 domain for these bispecific molecules.

TABLE 1

| Bispecific | Component | Amino Acid Sequence | DNA Sequence |
|---|---|---|---|
| V_{β}V_{α}-FTab-KiH | V_{β} - V_{α} | SEQ ID NO: 1 | SEQ ID NO: 41 |
| | Linker | GGGGSGGGGSGGGGSGGGGS | |
| | V_{α} - V_{H} | SEQ ID NO: 1 | SEQ ID NO: 41 |
| | Linker | | |
| | V_{β} | SEQ ID NO: 2 | SEQ ID NO: 42 |
| | | SQTIHQWPATLVQPVGSPLSLECTVE [symbol] LYWYRQAAGRCLELLFY [symbol] QISSEVPQN | |
| | | (CDR1; SEQ ID NO: 20) (CDR2; SEQ ID NO: 24) | |
| | | LSASRPQDRQFILSSKKLLLSDSGFYLC [symbol] FGPGTRLTVLEDLKD | |
| | | (CDR3; SEQ ID NO: 28) | |
| | V_{α} | SEQ ID NO: 6 | SEQ ID NO: 46 |
| | | QKEVEQNSGPLSVPEGAIASLNCTYS [symbol] FFWYRQYPGKSPELIMS [symbol] KEDGRFTA | |
| | | (CDR1; SEQ ID NO: 19) (CDR2; SEQ ID NO: 23) | |
| | | QLNKASQYVSLLIRDSQPSDSATYLC [symbol] FGCGTQLVVKPNIR | |
| | | (CDR3; SEQ ID NO: 27) | |
| | V_{H} | SEQ ID NO: 9 | SEQ ID NO: 49 |
| | | QVQLVQSGAEVKKPGASVKVSCKASGYTF [symbol] WVRQAPGQGLEWMG [symbol] | |
| | | (CDR1; SEQ ID NO: 21) (CDR2; SEQ ID NO: 25) | |
| | | [symbol] KATLTADKSASTAYMELSSLRSEDTAVYYCAR [symbol] WGQGTLVTVSS | |
| | | (CDR3; SEQ ID NO: 29) | |
| | V_{L} | SEQ ID NO: 11 | SEQ ID NO: 51 |
| | | DIQMTQSPSSLSASVGDRVTITC [symbol] WYQQKPGKAPKRLIY [symbol] GVPSRFSGS | |
| | | (CDR1; SEQ ID NO: 22) (CDR2; SEQ ID NO: 26) | |
| | | GSGTDFTLTISSLQPEDFATYYC [symbol] FGGGTKVEIKR | |
| | | (CDR3; SEQ ID NO: 30) | |
| | CH2CH3 | SEQ ID NO: 13 | SEQ ID NO: 53 |
| | | SVFLFPPKPKDTLMISRTPEVTCVVVDVSHEDPEVKFNWYVDGVEVHNAKTKPREEQYASTY | |
| | | RVVSVLTVLHQDWLNGKEYKCKVSNKALPAPIEKTISKAKGQPREPQVYTLPPSREEMTKNQ | |
| | | VSLSCAVKGFYPSDIAVEWESNGQPENNYKTTPPVLDSDGSFFLVSKLTVDKSRWQQGNVFSC | |
| | | SVMHEALHNHYTQKSLSLSPGK | |
| | CH2′CH3′ | SEQ ID NO: 16 | SEQ ID NO: 56 |
| | | DKTHTCPPCPAPELLGGPSVFLFPPKPKDTLMISRTPEVTCVVVDVSHEDPEVKFNWYVDGVE | |
| | | VHNAKTKPREEQYASTYRVVSVLTVLHQDWLNGKEYKCKVSNKALPAPIEKTISKAKGQPRE | |
| | | PQVYTLPPSREEMTKNQVSLWCLVKGFYPSDIAVEWESNGQPENNYKTTPPVLDSDGSFFLYS | |
| | | KLTVDKSRWQQGNVFSCSVMHEALHNHYTQKSLSLSPGK | |
| | C_{κ} | SEQ ID NO: 17 | SEQ ID NO: 57 |
| | | TVAAPSVFIFPPSDEQLKSGTASVVCLLNNFYPREAKVQWKVDNALQSGNSQESVTEQDSKDS | |
| | | TYSLSSTLTLSKADYEKHKVYACEVTHQGLSSPVTKSFNRGEC | |
| | CH1 | SEQ ID NO: 18 | SEQ ID NO: 58 |
| | | ASTKGPSVFPLAPSSKSTSGGTAALGCLVKDYFPEPVTVSWNSGALTSGVHTFPAVLQSSGLYS | |
| | | LSSVVTVPSSSLGTQTYICNVNHKPSNTKVDKKVEPKSCDKTHTCPPCPAPELLGGP | |
| | CH1CH2CH3 | SEQ ID NO: 37 | SEQ ID NO: 77 |
| | | ASTKGPSVFPLAPSSKSTSGGTAALGCLVKDYFPEPVTVSWNSGALTSGVHTFPAVLQSSGLYS | |
| | | LSSVVTVPSSSLGTQTYICNVNHKPSNTKVDKKVEPKSCDKTHTCPPCPAPELLGGPSVFLFPPK | |
| | | PKDTLMISRTPEVTCVVVDVSHEDPEVKFNWYVDGVEVHNAKTKPREEQYASTYRVVSVLTV | |
| | | LHQDWLNGKEYKCKVSNKALPAPIEKTISKAKGQPREPQVYTLPPSREEMTKNQVSLSCAVK | |
| | | GFYPSDIAVEWESNGQPENNYKTTPPVLDSDGSFFLVSKLTVDKSRWQQGNVFSCSVMHEAL | |
| | | HNHYTQKSLSLSPGK | |
| | Heavy | SEQ ID NO: 36 | SEQ ID NO: 80 |
| | Chain 1 | SQTIHQWPATLVQPVGSPLSLECTVEGTSNPNLYWYRQAAGRCLELLFYSVGIGQISSEVPQNL | |
| | | SASRPQDRQFILSSKKLLLSDSGFYLCAWSIGAEMFFGPGTRLTVLEDLKDGGGGSGGGGSGG | |
| | | GGSGGGGSQKEVEQNSGPLSVPEGAIASLNCTYSDRYAQNFFWYRQYPGKSPELIMSIYSNGD | |
| | | KEDGRFTAQLNKASQYVSLLIRDSQPSDSATYLCAVSKGYKVFGCGTQLVVKPNIRGGGGSG | |
| | | GGGSGGGGSGGGGSQVQLVQSGAEVKKPGASVKVSCKASGYTFISYTMHWVRQAPGQGLE | |
| | | WMGYINPRSGYTHYNQKLKDKATLTADKSASTAYMELSSLRSEDTAVYYCARSAYYDYDGF | |
| | | AYWGQGTLVTVSSASTKGPSVFPLAPSSKSTSGGTAALGCLVKDYFPEPVTVSWNSGALTSGV | |
| | | HTFPAVLQSSGLYSLSSVVTVPSSSLGTQTYICNVNHKPSNTKVDKKVEPKSCDKTHTCPPCPA | |
| | | PELLGGPSVFLFPPKPKDTLMISRTPEVTCVVVDVSHEDPEVKFNWYVDGVEVHNAKTKPREE | |
| | | QYASTYRVVSVLTVLHQDWLNGKEYKCKVSNKALPAPIEKTISKAKGQPREPQVYTLPPSREE | |
| | | MTKNQVSLSCAVKGFYPSDIAVEWESNGQPENNYKTTPPVLDSDGSFFLVSKLTVDKSRWQQ | |
| | | GNVFSCSVMHEALHNHYTQKSLSLSPGK | |
| | Heavy | SEQ ID NO: 16 | SEQ ID NO: 56 |
| | Chain 2 | DKTHTCPPCPAPELLGGPSVFLFPPKPKDTLMISRTPEVTCVVVDVSHEDPEVKFNWYVDGVE | |
| | | VHNAKTKPREEQYASTYRVVSVLTVLHQDWLNGKEYKCKVSNKALPAPIEKTISKAKGQPRE | |
| | | PQVYTLPPSREEMTKNQVSLWCLVKGFYPSDIAVEWESNGQPENNYKTTPPVLDSDGSFFLYS | |
| | | KLTVDKSRWQQGNVFSCSVMHEALHNHYTQKSLSLSPGK | |
| | Light | SEQ ID NO: 76 | SEQ ID NO: 87 |
| | Chain | DIQMTQSPSSLSASVGDRVTITCSASSSVSYMNWYQQKPGKAPKRLIYDTSKLASGVPSRFSGS | |
| | | GSGTDFTLTISSLQPEDFATYYCQQWSSNPPTFGGGTKVEIKRTVAAPSVFIFPPSDEQLKSGTA | |
| | | SVVCLLNNFYPREAKVQWKVDNALQSGNSQESVTEQDSKDSTYSLSSTLTLSKADYEKHKVY | |
| | | ACEVTHQGLSSPVTKSFNRGEC | |
| V_{β}V_{α}-FTab-KiH-2 | V_{β} -V_{α} | SEQ ID NO: 1 | SEQ ID NO: 41 |
| | Linker | | |
| | V_{α} - V_{H} | SEQ ID NO: 1 | SEQ ID NO: 41 |
| | Linker | | |
| | V_{β} | SEQ ID NO: 5 | SEQ ID NO: 45 |
| | | SQTIHQWPATLVQPVGSPLSLECTVE [symbol] LYWYRQAAGRCLELLFY [symbol] QISSEVPQN | |
| | | (CDR1; SEQ ID NO: 20) (CDR2; SEQ ID NO: 24) | |
| | | LSASRPQDRQFILSSKKLLLSDSGFYLC [symbol] FGPGTRLTVLEDLKN | |
| | | (CDR3; SEQ ID NO: 28) | |
| | V_{α} | SEQ ID NO: 6 | SEQ ID NO: 46 |
| | V_{H} | SEQ ID NO: 9 | SEQ ID NO: 49 |
| | V_{L} | SEQ ID NO: 11 | SEQ ID NO: 51 |
| | CH2CH3 | SEQ ID NO: 13 | SEQ ID NO: 53 |
| | CH2′CH3′ | SEQ ID NO: 16 | SEQ ID NO: 56 |
| | C_{κ} | SEQ ID NO: 17 | SEQ ID NO: 57 |
| | CH1 | SEQ ID NO: 18 | SEQ ID NO: 58 |
| | CH1CH2CH3 | SEQ ID NO: 37 | SEQ ID NO: 77 |
| | Heavy | SEQ ID NO: 88 | SEQ ID NO: 89 |
| | Chain 1 | SQTIHQWPATLVQPVGSPLSLECTVEGTSNPNLYWYRQAAGRCLELLFYSVGIGQISSEVPQNL | |
| | | SASRPQDRQFILSSKKLLLSDSGFYLCAWSIGAEMFFGPGTRLTVLEDLKNGGGGSGGGGSGG | |
| | | GGSGGGGSQKEVEQNSGPLSVPEGAIASLNCTYSDRYAQNFFWYRQYPGKSPELIMSIYSNGD | |
| | | KEDGRFTAQLNKASQYVSLLIRDSQPSDSATYLCAVSKGYKVFGCGTQLVVKPNIRGGGGSG | |
| | | GGGSGGGGSGGGGSQVQLVQSGAEVKKPGASVKVSCKASGYTFISYTMHWVRQAPGQGLE | |
| | | WMGYINPRSGYTHYNQKLKDKATLTADKSASTAYMELSSLRSEDTAVYYCARSAYYDYDGF | |
| | | AYWGQGTLVTVSSASTKGPSVFPLAPSSKSTSGGTAALGCLVKDYFPEPVTVSWNSGALTSGV | |
| | | HTFPAVLQSSGLYSLSSVVTVPSSSLGTQTYICNVNHKPSNTKVDKKVEPKSCDKTHTCPPCPA | |
| | | PELLGGPSVFLFPPKPKDTLMISRTPEVTCVVVDVSHEDPEVKFNWYVDGVEVHNAKTKPREE | |
| | | QYASTYRVVSVLTVLHQDWLNGKEYKCKVSNKALPAPIEKTISKAKGQPREPQVYTLPPSREE | |
| | | MTKNQVSLSCAVKGFYPSDIAVEWESNGQPENNYKTTPPVLDSDGSFFLVSKLTVDKSRWQQ | |
| | | GNVFSCSVMHEALHNHYTQKSLSLSPGK | |
| | Heavy | SEQ ID NO: 16 | SEQ ID NO: 56 |
| | Chain 2 | | |
| | Light | SEQ ID NO: 76 | SEQ ID NO: 87 |
| | Chain | | |
| V_{β}V_{α}-FTab-hb-1 | V_{β} - V_{α} | SEQ ID NO: 1 | SEQ ID NO: 41 |
| | Linker | | |
| | V_{α} - V_{H} | SEQ ID NO: 1 | SEQ ID NO: 41 |
| | Linker | | |
| | V_{β} | SEQ ID NO: 3 | SEQ ID NO: 43 |
| | | SQTIHQWPATLVQPVGSPLSLECTVE [symbol] LYWYRQAAGRGPELLFY [symbol] QISSEVPQN | |
| | | (CDR1; SEQ ID NO: 20) (CDR2; SEQ ID NO: 24) | |
| | | LFASRPQDRQFILSSKKLLLSDSGFYLC [symbol] FGPGTRLTVLEDLKN | |
| | | (CDR3; SEQ ID NO: 31) | |
| | V_{α} | SEQ ID NO: 7 | SEQ ID NO: 47 |
| | | QKEVEQNSGPLSVPEGAIASLNCTYS [symbol] FFWYRQYSGKSPELIMS [symbol] KEDGRFTA | |
| | | (CDR1; SEQ ID NO: 19) (CDR2; SEQ ID NO: 23) | |
| | | QLNKASQYVSLLIRDSQPSDSATYLC [symbol] FGDGTQLVVKPNIR | |
| | | (CDR3; SEQ ID NO: 27)) | |
| | V_{H} | SEQ ID NO: 9 | SEQ ID NO: 49 |
| | V_{L} | SEQ ID NO: 11 | SEQ ID NO: 51 |
| | CH2CH3 | SEQ ID NO: 14 | SEQ ID NO: 54 |
| | | CVFLFPPKPKDTLMISRTPEVTCVVVDVSHEDPEVKFNWYVDGVEVHNAKTKPREEQYNSTY | |
| | | RVVSVLTVLHQDWLNGKEYKCKVSNKALPAPIEKTISKAKGQPREPQVYTLPPSREEMTKNQ | |
| | | VSLTCLVKGFYPSDIAVEWESNGQPENNYKTTAPVLDSDGSFRLRSDLTVDKSRWQQGNVFS | |
| | | CSVMHEALHNHYTQKSLSLSPGK | |
| | C_{κ} | SEQ ID NO: 17 | SEQ ID NO: 57 |
| | CH1 | SEQ ID NO: 35 | SEQ ID NO: 75 |
| | | ASTKGPSVFPLAPSSKSTSGGTAALGCLVKDYFPEPVTVSWNSGALTSGVHTFPAVLQSSGLYS | |
| | | LSSVVTVPSSSLGTQTYICNVNHKPSNTKVDKKVEPKSSDKTHTSPPCPAPELLGGP | |
| | CH1CH2CH3 | SEQ ID NO: 38 | SEQ ID NO: 78 |
| | | ASTKGPSVFPLAPSSKSTSGGTAALGCLVKDYFPEPVTVSWNSGALTSGVHTFPAVLQSSGLYS | |
| | | LSSVVTVPSSSLGTQTYICNVNHKPSNTKVDKKVEPKSSDKTHTSPPCPAPELLGGPCVFLFPPK | |
| | | PKDTLMISRTPEVTCVVVDVSHEDPEVKFNVVYVDGVEVHNAKTKPREEQYNSTYRVVSVLTV | |
| | | LHQDWLNGKEYKCKVSNKALPAPIEKTISKAKGQPREPQVYTLPPSREEMTKNQVSLTCLVKG | |
| | | FYPSDIAVEWESNGQPENNYKTTAPVLDSDGSFRLRSDLTVDKSRWQQGNVFSCSVMHEALH | |
| | | NHYTQKSLSLSPGK | |
| V_{α}V_{β}-FTab-hb-1 | V_{α} - V_{β} | SEQ ID NO: 1 | SEQ ID NO: 41 |
| | Linker | | |
| | V_{β} - V_{H} | SEQ ID NO: 1 | SEQ ID NO: 41 |
| | Linker | | |
| | V_{β} | SEQ ID NO: 3 | SEQ ID NO: 43 |
| | V_{α} | SEQ ID NO: 7 | SEQ ID NO: 47 |
| | V_{H} | SEQ ID NO: 9 | SEQ ID NO: 49 |
| | V_{L} | SEQ ID NO: 11 | SEQ ID NO: 51 |
| | CH2CH3 | SEQ ID NO: 14 | SEQ ID NO: 54 |
| | C_{κ} | SEQ ID NO: 17 | SEQ ID NO: 57 |
| | CH1 | SEQ ID NO: 35 | SEQ ID NO: 75 |
| | CH1CH2CH3 | SEQ ID NO: 38 | SEQ ID NO: 78 |
| V_{α}V_{β}-FTab | V_{α} - V_{β} | SEQ ID NO: 1 | SEQ ID NO: 41 |
| | Linker | | |
| | V_{β} - V_{H} | SEQ ID NO: 1 | SEQ ID NO: 41 |
| | Linker | | |
| | V_{β} | SEQ ID NO: 3 | SEQ ID NO: 43 |
| | V_{α} | SEQ ID NO: 7 | SEQ ID NO: 47 |
| | V_{H} | SEQ ID NO: 9 | SEQ ID NO: 49 |
| | V_{L} | SEQ ID NO: 11 | SEQ ID NO: 51 |
| | C_{κ} | SEQ ID NO: 17 | SEQ ID NO: 57 |
| | CH1 | SEQ ID NO: 18 | SEQ ID NO: 58 |
| V_{β}V_{α}-FTab-1 | V_{β} - V_{α} | SEQ ID NO: 1 | SEQ ID NO: 41 |
| | Linker | | |
| | V_{α} - V_{H} | SEQ ID NO: 1 | SEQ ID NO: 41 |
| | Linker | | |
| | V_{β} | SEQ ID NO: 3 | SEQ ID NO: 43 |
| | V_{α} | SEQ ID NO: 7 | SEQ ID NO: 47 |
| | V_{H} | SEQ ID NO: 9 | SEQ ID NO: 49 |
| | V_{L} | SEQ ID NO: 11 | SEQ ID NO: 51 |
| | C_{κ} | SEQ ID NO: 17 | SEQ ID NO: 57 |
| | CH1 | SEQ ID NO: 18 | SEQ ID NO: 58 |
| V_{β}V_{α}-FTab-hb-2 | V_{β} - V_{α} | SEQ ID NO: 1 | SEQ ID NO: 41 |
| | Linker | | |
| | V_{α} - V_{H} | SEQ ID NO: 1 | SEQ ID NO: 41 |
| | Linker | | |
| | V_{β} | SEQ ID NO: 4 | SEQ ID NO: 44 |
| | | SQTIHQWPATLVQPVGSPLSLECTVE [symbol] LYWYRQAAGRCLELLFY [symbol] QISSEVPQN | |
| | | (CDR1; SEQ ID NO: 20) (CDR2; SEQ ID NO: 24) | |
| | | LFASRPQDRQFILSSKKLLLSDSGFYLC [symbol] FGPGTRLTVLEDLKN | |
| | | (CDR3; SEQ ID NO: 31) | |
| | V_{α} | SEQ ID NO: 8 | SEQ ID NO: 48 |
| | | QKEVEQNSGPLSVPEGAIASLNCTYS [symbol] FFWYRQYSGKSPELIMS [symbol] KEDGRETA | |
| | | (CDR1; SEQ ID NO: 19) (CDR2; SEQ ID NO: 23) | |
| | | QLNKASQYVSLLIRDSQPSDSATYLC [symbol] FGCGTQLVVKPNIR | |
| | | (CDR3; SEQ ID NO: 27) | |
| | V_{H} | SEQ ID NO: 9 | SEQ ID NO: 49 |
| | V_{L} | SEQ ID NO: 11 | SEQ ID NO: 51 |
| | CH2CH3 | SEQ ID NO: 14 | SEQ ID NO: 54 |
| | C_{κ} | SEQ ID NO: 17 | SEQ ID NO: 57 |
| | CH1 | SEQ ID NO: 35 | SEQ ID NO: 75 |
| | CH1CH2CH3 | SEQ ID NO: 38 | SEQ ID NO: 78 |
| V_{β}V_{α}-FTab-hb-3 | V_{β} - V_{α} | SEQ ID NO: 1 | SEQ ID NO: 41 |
| | Linker | | |
| | V_{α} - V_{H} | SEQ ID NO: 1 | SEQ ID NO: 41 |
| | Linker | | |
| | V_{β} | SEQ ID NO: 5 | SEQ ID NO: 45 |
| | V_{α} | SEQ ID NO: 6 | SEQ ID NO: 46 |
| | V_{H} | SEQ ID NO: 9 | SEQ ID NO: 49 |
| | V_{L} | SEQ ID NO: 11 | SEQ ID NO: 51 |
| | CH2CH3 | SEQ ID NO: 14 | SEQ ID NO: 54 |
| | C_{κ} | SEQ ID NO: 17 | SEQ ID NO: 57 |
| | CH1 | SEQ ID NO: 35 | SEQ ID NO: 75 |
| | CH1CH2CH3 | SEQ ID NO: 38 | SEQ ID NO: 78 |
| V_{β}V_{α}-FTab-hb-4 | V_{β} - V_{α} | SEQ ID NO: 1 | SEQ ID NO: 41 |
| | Linker | | |
| | V_{α} - V_{H} | SEQ ID NO: 1 | SEQ ID NO: 41 |
| | Linker | | |
| | V_{β} | SEQ ID NO: 5 | SEQ ID NO: 45 |
| | V_{α} | SEQ ID NO: 6 | SEQ ID NO: 46 |
| | V_{H} | SEQ ID NO: 10 | SEQ ID NO: 50 |
| | | EVQLVESGGGLVQPGGSLRLSCAAS [symbol] WVRQAPGKGLEWVA [symbol] TY | ORF Start: 1 |
| | | (CDR1; SEQ ID NO: 32) (CDR2; SEQ ID NO: 33) | ORF Stop: 366 |
| | | ADSVKGRFTISVDKSKNTAYLQMNSLRAEDTAVYYCAR [symbol] WGQGTLVTV | |
| | | (CDR3; SEQ ID NO: 34) | |
| | | SS | |
| | V_{L} | SEQ ID NO: 12 | SEQ ID NO: 52 |
| | | DIQMTQSPSSLSASVGDRVTITC [symbol] WYQQKPGKAPKLLIY [symbol] GVPSRFS | |
| | | (CDR1; SEQ ID NO: 81) (CDR2; SEQ ID NO: 82) | |
| | | GSGSGTDYTLTISSLQPEDFATYYC [symbol] FGQGTKVEIKR | |
| | | (CDR3; SEQ ID NO: 83) | |
| | CH2CH3 | SEQ ID NO: 14 | SEQ ID NO: 54 |
| | C_{κ} | SEQ ID NO: 17 | SEQ ID NO: 57 |
| | CH1 | SEQ ID NO: 35 | SEQ ID NO: 75 |
| | CH1CH2CH3 | SEQ ID NO: 38 | SEQ ID NO: 78 |
| V_{β}V_{α}-FTab-2 | V_{β} - V_{α} | SEQ ID NO: 1 | SEQ ID NO: 41 |
| | Linker | | |
| | V_{α} - V_{H} | SEQ ID NO: 1 | SEQ ID NO: 41 |
| | Linker | | |
| | V_{β} | SEQ ID NO: 5 | SEQ ID NO: 45 |
| | V_{α} | SEQ ID NO: 6 | SEQ ID NO: 46 |
| | V_{H} | SEQ ID NO: 9 | SEQ ID NO: 49 |
| | V_{L} | SEQ ID NO: 11 | SEQ ID NO: 51 |
| | C_{κ} | SEQ ID NO: 17 | SEQ ID NO: 57 |
| | CH1 | SEQ ID NO: 18 | SEQ ID NO: 58 |
| V_{β}V_{α}-FTab-3 | V_{β} - V_{α} | SEQ ID NO: 1 | SEQ ID NO: 41 |
| | Linker | | |
| | V_{α} - V_{H} | SEQ ID NO: 1 | SEQ ID NO: 41 |
| | Linker | | |
| | V_{β} | SEQ ID NO: 5 | SEQ ID NO: 45 |
| | V_{α} | SEQ ID NO: 6 | SEQ ID NO: 46 |
| | V_{H} | SEQ ID NO: 10 | SEQ ID NO: 50 |
| | V_{L} | SEQ ID NO: 12 | SEQ ID NO: 52 |
| | C_{κ} | SEQ ID NO: 17 | SEQ ID NO: 57 |
| | CH1 | SEQ ID NO: 18 | SEQ ID NO: 58 |
| V_{β}V_{α}-FTab-hb-5 | V_{β} - V_{α} | SEQ ID NO: 1 | SEQ ID NO: 41 |
| | Linker | | |
| | V_{α} - V_{H} | SEQ ID NO: 1 | SEQ ID NO: 41 |
| | Linker | | |
| | V_{β} | SEQ ID NO: 5 | SEQ ID NO: 45 |
| | V_{α} | SEQ ID NO: 6 | SEQ ID NO: 46 |
| | V_{H} | SEQ ID NO: 9 | SEQ ID NO: 49 |
| | V_{L} | SEQ ID NO: 11 | SEQ ID NO: 51 |
| | CH2CH3 | SEQ ID NO: 15 | SEQ ID NO: 55 |
| | | SVFLFPPKPKDTLMISRTPEVTCVVVDVSHEDPEVKFNWYVDGVEVHNAKTKPREEQYASTY | |
| | | RVVSVLTVLHQDWLNGKEYKCKVSNKALPAPIEKTISKAKGQPREPQVYTLPPSRDEMTKNQ | |
| | | VSLTCLVKGFYPSDIAVEWESNGQPENNYKTTPPVLDSDGSFRLYSKLTVDKSRWQQGNVFSC | |
| | | SVMHEALHNHYTQKSLSLSPGK | |
| | C_{κ} | SEQ ID NO: 17 | SEQ ID NO: 57 |
| | CH1 | SEQ ID NO: 35 | SEQ ID NO: 75 |
| | CH1CH2CH3 | SEQ ID NO: 39 | SEQ ID NO: 79 |
| | | ASTKGPSVFPLAPSSKSTSGGTAALGCLVKDYFPEPVTVSWNSGALTSGVHTFPAVLQSSGLYS | |
| | | LSSVVTVPSSSLGTQTYICNVNHKPSNTKVDKKVEPKSSDKTHTSPPCPAPELLGGPSVFLFPPK | |
| | | PKDTLMISRTPEVTCVVVDVSHEDPEVKFNVVYVDGVEVHNAKTKPREEQYASTYRVVSVLTV | |
| | | LHQDWLNGKEYKCKVSNKALPAPIEKTISKAKGQPREPQVYTLPPSRDEMTKNQVSLTCLVK | |
| | | GFYPSDIAVEWESNGQPENNYKTTPPVLDSDGSFRLYSKLTVDKSRWQQGNVFSCSVMHEAL | |
| | | HNHYTQKSLSLSPGK | |

### Example 2: Expression and Purification

Plasmid DNA was provided internally, and protein was expressed in HEK293-6E cells using a transient transfection method. 0.5 mg DNA per liter cell culture was transfected into HEK293-6E cells at a density of 1.4×10⁶ cells/mL using Polyethylenimine Max (PEI Max, Polysciences Inc) at a PEI:DNA ratio of 4:1 and Light Chain:Heavy Chain DNA ratio of 3:2. HEK293-6E cells were grown in FreeStyle™ 293 medium (Invitrogen) in suspension with 5% CO₂ at 37° C., in 2.8 L shaking flasks (125 RPM). Cells were fed with 0.5% Tryptone N1 one day after transfection. On day 7 post-transfection, the transfected cell cultures were cleared by centrifugation followed by filtration through 0.2 μm PES filter (Corning).

Expression of bispecific molecules: All bispecific proteins of Example 1 were expressed in HEK293-6E cells using a transient transfection method. 0.5 mg DNA per liter cell culture was transfected into HEK293-6E cells at a density of 1.4×10⁶ cells/mL using Polyethylenimine Max (PEI Max, Polysciences Inc) at a PEI:DNA ratio of 4:1 and Light Chain:Heavy Chain 1:Heavy Chain 2 DNA ratio of 1:1:1. HEK293-6E cells were grown in FreeStyle™ 293 medium (Invitrogen) in suspension with 5% CO₂ at 37° C., in a 10 L Wave bag (28 RPM, 7Angle). Cells were fed with 0.5% Tryptone N1 one day after transfection. On day 7 post-transfection, the transfected cell culture was cleared by centrifugation followed by filtration through 0.45/0.2 μm filter (Sartorius Stedim).

Purification of V_{β}V_{α}-FTab-hb-1, V_{β}V_{α}-FTab-hb-2, V_{β}V_{α}-FTab-hb-3, V_{β}V_{α}-FTab-hb-4, V_{β}V_{α}-FTab-hb-5, V_{β}V_{α}-FTab-KiH and V_{β}V_{α}-FTab-KiH-2: Cleared medium was loaded on a MabSelect SuRe™ column (GE Healthcare) equilibrated with PBS, pH 7.4. The column was washed with PBS, pH 7.4 and bound protein was eluted with 0.1M acetic acid pH 2.7, 0.15M NaCl. Fractions were neutralized with 1M Tris pH 9.0 at a ratio of 1:10. Neutralized protein was further purified by SEC on a Superdex 200 column (GE Healthcare) equilibrated and run with PBS, pH 7.4. Fractions containing protein were pooled, concentration was measured by absorbance at 280 nm, and samples were analyzed by SEC, SDS-PAGE, and mass spectrometry. The final material was stored in aliquots at −80° C.

Purification of V_{β}V_{α}-FTab-1, V_{β}V_{α}-FTab-2, V_{β}V_{α}-FTab-3 and V_{α}V_{β}-FTab: Cleared medium was buffer exchanged to PBS, pH 7.4 using a Kvick™ TFF system equipped with 10 kDa membranes (GE Healthcare) and loaded on a HisTrap™ FF column (GE Healthcare) equilibrated with PBS, pH 7.4. The column was washed with 25 mm imidazole in PBS, pH 7.4 and bound protein was eluted with 250 mM imidazole in PBS, pH 7.4. Eluted protein was further purified by SEC on a Superdex® 200 column (GE Healthcare), equilibrated, and run with PBS, pH 7.4. Fractions containing anti-CD3-Fab were pooled, concentration was measured by absorbance at 280 nm, and samples were analyzed by SEC, SDS-PAGE, and mass spectrometry. Final material was stored in aliquots at −80° C.

### Example 3: Assays and Characterization

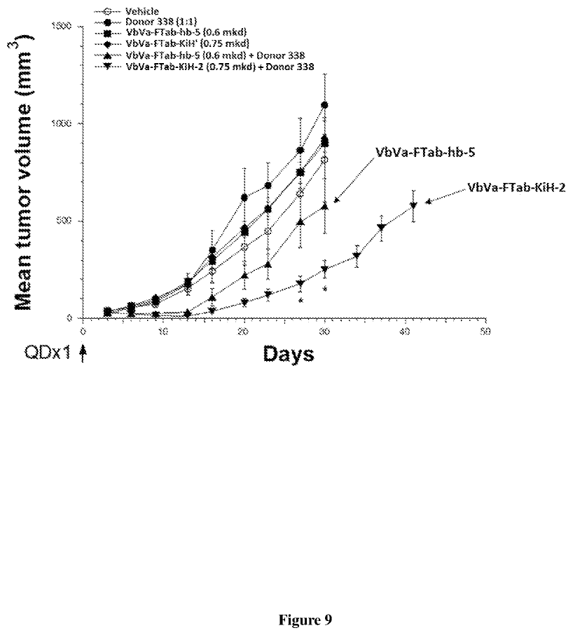

The bispecific molecules with a Fc region that is either a dimeric knob-in-hole (e.g., V_{β}V_{α}-FTab KiH′ which was also designated as V_{β}V_{α}-FTab KiH-2) or a halfbody (e.g., V_{β}V_{α}-FTab-hb-1) exhibited improved pharmacokinetics and serum stability properties while maintaining potency and specificity through monovalent binding to both SURV/HLA-A2 and CD3. As shown in FIG. 9, in a single dose comparison study in the HCT-116 CRC ES model, it was unexpectedly discovered that at molar equivalent doses, the bispecific molecule containing KiH (V_{β}V_{α}-FTab-KiH-2) exhibited greater anti-tumor efficacy than V_{β}V_{α}-FTab-hb-5. V_{β}V_{α}-FTab-KiH-2 is almost identical to V_{β}V_{α}-FTab-KiH except for one amino acid substitution to mitigate deamidation, which is not expected to have any impact on potency.

### Example 4: Target Cell Labeling for AML Cell Line Functional Assays

Cell lines OCI-AML2 (ACC-99), OCI-AML3 (ACC-582), and OCI-Ly19 (ACC-528) were purchased from DSMZ and were cultured in α-MEM supplemented with 20% FBS and incubated at 37° C. and 5% CO₂. OCI-M1 (ACC-529) was also purchased from DSMZ and cultured in IMDM supplemented with 10% FBS and incubated at 37° C. and 5% CO₂. Cells were stained with CellVue™ Burgundy (Invitrogen) prior to co-culture with T cells. Target cells were pelleted and washed with PBS once. Cells were resuspended in Diluent C per manufacturer instructions and incubated with a final concentration of 2 μM Burgundy CellVue™ dye for 5 minutes at room temperature in the dark. The reaction was stopped by adding equal volume of FBS (Sigma). Samples were washed 3 times with cell-line specific complete medium. Cells were counted and checked for efficiency of labeling by FACS, prior to seeding into functional assays. The APC-Cy7 channel was used to detect CellVue™ Burgundy signal.

### Example 5: Effector T Cell Labeling for Functional Assays

Effector T cells were isolated from donor PBMC stocks by negative selection using a T cell isolation kit (Miltenyi) on LS columns (Miltenyi). MACS™ buffer (PBS supplemented with 0.1% BSA and 2 mM EDTA) was used for isolation of CD3+ T cells. Isolated CD3+ T cells were cultured in AIM V™ media supplemented with 5% AB serum and incubated at 37° C. and 5% CO₂ overnight. The following day, cells were counted and labeled with CellTrace™ Violet (Invitrogen). Effector cells were pelleted and washed once with PBS. Effector cells were aliquoted 10′ per 50 mL tube in 10 mL PBS. CellTrace™ stock solution was prepared immediately prior to use by adding the 20 μL volume of DMSO (Component B) to one vial of CellTrace™ reagent (Component A) and mixing well. Ten microliters of CellTrace™ reagent was added to each 50 mL tube containing effector cells. Effector cells were stained for 20 minutes at 37° C. and 5% CO₂ and shaken sporadically to ensure efficient staining. To stop the reaction, 40 mL of AIM V™ supplemented with 10% FBS was added to each 50 mL tube. Reaction blocking took 5 minutes at room temperature in the dark; cells were pelleted and resuspended with AIM V™ media supplemented with 5% AB serum. Cells were counted and checked for efficiency of labeling by FACS, prior to seeding into functional assays. The Pacific Blue channel was used to detect CellTrace™ Violet signal.

### Example 6: Redirected T Cell Cytotoxicity and Activation Assays

CellVue™ Burgundy-labeled target cells were seeded at 20,000 cells per well into a round bottom 96-well plate (BD) in 50 μl volume per well. CellTrace™ Violet-labeled effector T cells were added to appropriate wells (in duplicate) at 200,000 cells per well in 50 μl volume, for approximate Effector T-cell/Target ratio (E:T) of 10:1. Serially diluted Survivin TCR/CD3 bispecific molecule was added to appropriate wells in a 50 μl volume, starting at 6 nM per well and titrated in a 3-fold dilution across 9 wells (in duplicate). The mixed cultures were placed at 37° C. and 5% CO₂ for 48 hours. Target cytotoxicity and T cell activation parameters were found to be optimal at 48 hours. At the time of the harvest, the culture supernatant was collected for cytokine release analysis while the cells were pelleted and stained with FACS antibodies to detect target cytotoxicity, T cell activation, and T cell proliferation. Briefly, the 96-well plates containing samples were palleted and washed twice with FACS buffer (PBS supplemented with 0.5% BSA and 2 mM EDTA). Antibodies against T cell activation markers CD25-PE (Biolegend), CD69-APC (Biolegend), and CD3-PE-Cy7 (Biolegend) were mixed at 7.5 μl/ml FACS buffer and 25 μl were added per well. Samples were allowed to incubate for 25 minutes at 4° C. in the dark. Samples were washed twice with FACS buffer. Viability dyes Annexin-FITC (Biolegend) and 7AAD (Biolegend) were mixed in Annexin V binding buffer (Biolegend) at 7.5 μl/ml and 15 μl/ml, respectively, and added to wells at 25 μl/well for 15 minutes at room temperature in the dark. At the end of the incubation, 75 μl of Annexin V binding buffer was added to each well. Data was acquired on FACSCanto II™ and analyzed using FlowJo™ V10 analysis software. The dose-response data for target cytotoxicity, T cell activation and T cell proliferation were fitted to a sigmoidal curve using nonlinear regression, and the EC₅₀ values calculated with the aid of GraphPad 5.0 Software.

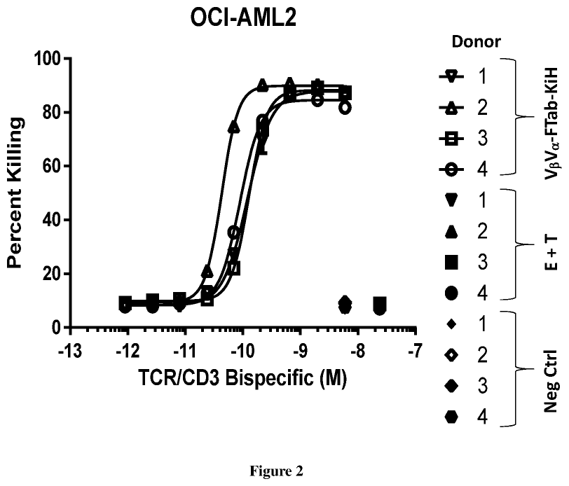

As shown in FIG. 2, V_{β}V_{α}-FTab-KiH was evaluated for its ability to redirect killing by CD3+ T cells against the HLA-A2, Survivin-positive AML cell line OCI-AML2. V_{β}V_{α}-FTab-KiH induced potent killing of OCI-AML2 across 4 healthy CD3+ T cell donors, while no activity was observed with a negative control (irrelevant TCR/CD3 bispecific) (Neg Ctrl).

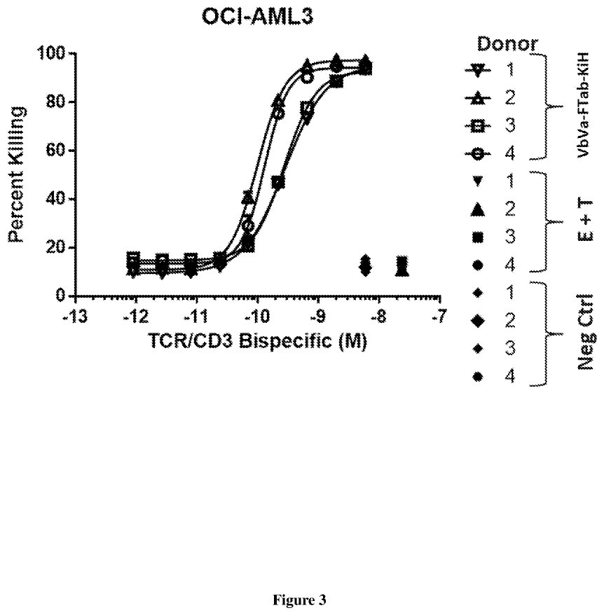

As shown in FIG. 3, V_{β}V_{α}-FTab-KiH was evaluated for its ability to redirect killing by CD3+ T cells against the HLA-A2, Survivin-positive AML cell line OCI-AML3. V_{β}V_{α}-FTab-KiH induced potent killing of OCI-AML3 across 4 healthy CD3+ T cell donors, while no activity was observed with an irrelevant TCR/CD3 bispecific (Neg Ctrl).

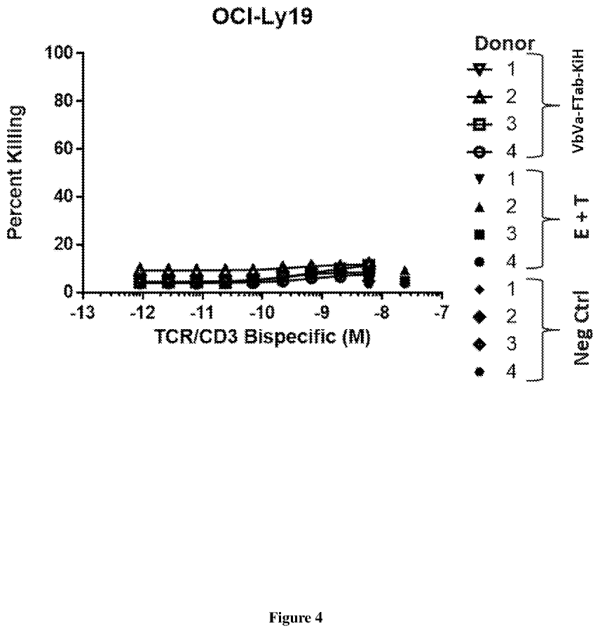

As shown in FIG. 4, V_{β}V_{α}-FTab-KiH was evaluated for its ability to redirect killing by CD3+ T cells against the HLA-A2 negative, Survivin-positive AML cell line OCI-Ly19. V_{β}V_{α}-FTab-KiH did not induce killing of OCI-Ly19, due to the lack of HLA-A2 expression by this cell line.

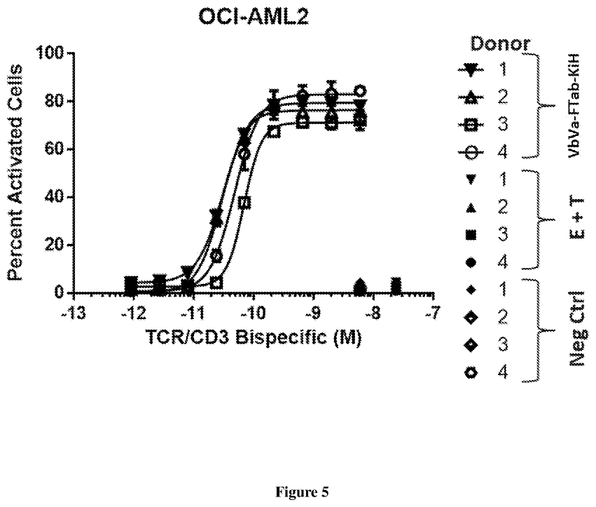

As shown in FIG. 5, V_{β}V_{α}-FTab-KiH was evaluated for its ability to activate CD3+ T cells against the HLA-A2, Survivin-positive AML cell line OCI-AML2, as measured by CD69 expression. V_{β}V_{α}-FTab-KiH induced potent activation of CD3+ T cells across 4 healthy CD3+ T cell donors, against OCI-AML2, while no activity was observed with an irrelevant TCR/CD3 bispecific (Neg Ctrl).

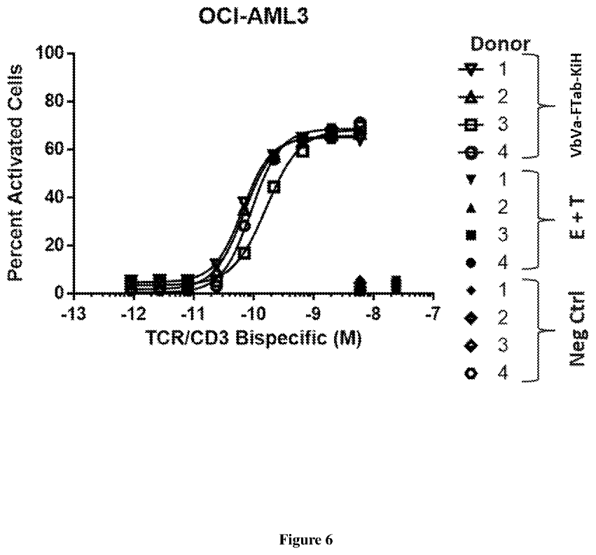

As shown in FIG. 6, V_{β}V_{α}-FTab-KiH was evaluated for its ability to activate CD3+ T cells against the HLA-A2, Survivin-positive AML cell line OCI-AML3, as measured by CD69 expression. V_{β}V_{α}-FTab-KiH induced potent activation of CD3+ T cells across 4 healthy CD3+ T cell donors, against OCI-AML3, while no activity was observed with an irrelevant TCR/CD3 bispecific (Neg Ctrl).

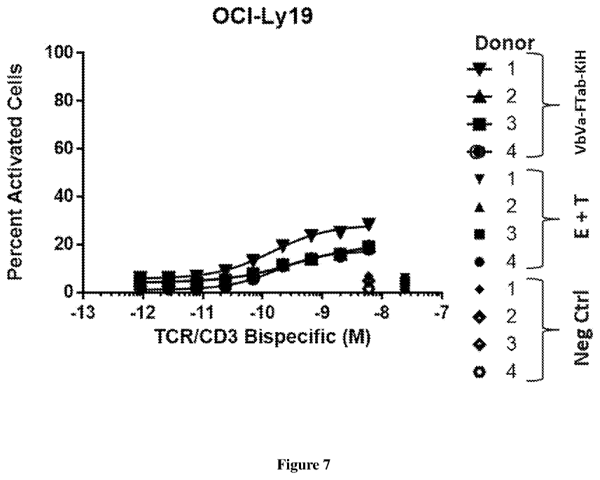

As shown in FIG. 7, V_{β}V_{α}-FTab-KiH was evaluated for its ability to activate CD3+ T cells against the HLA-A2 negative, Survivin-positive AML cell line OCI-Ly19, as measured by CD69 expression. V_{β}V_{α}-FTab-KiH induced minimal activation of CD3+ T cells across 4 healthy CD3+ T cell donors, against OCI-Ly19, due to the lack of HLA-A2 expression by this cell line.

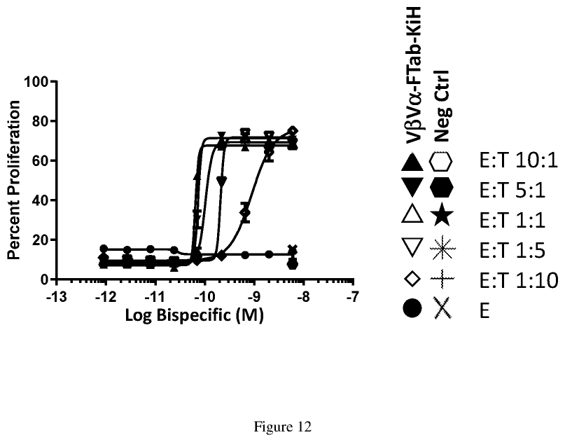

As shown in FIG. 12, V_{β}V_{α}-FTab-KiH was evaluated for its ability to induce T cell proliferation at varying effector to target ratios. V_{β}V_{α}-FTab-KiH induced T cell proliferation at varying effector to target ratios.

### Example 7: Pharmacokinetic Characterization of Survivin TCR/CD3 Bispecific Molecules

The pharmacokinetic profiles of Survivin/CD3 bispecific molecules were compared in non-tumor bearing SCID mice using a single 16 milligrams/kilogram (mpk) IV bolus dose. Whole blood samples were collected for both early and later time points (until 168 hours) for V_{β}V_{α}-FTab-KiH. Other molecules were analyzed for up to 48 hours. Analyte concentration was determined by a Meso Scale Discovery (MSD)-based assay with goat anti-human IgG-Fc as the capture reagent and goat anti-sulfate as the detection reagent. The half-life (t_{1/2}), area under the curve (AUC), clearance (CL) and steady state volume (Vss) values for all test molecules are summarized in Table 2. Results indicated that V_{β}V_{α}-FTab-KiH exhibited antibody-like pharmacokinetics with a surprisingly longer half-life (˜5 days) and higher exposure as compared to other molecules tested.

TABLE 2
Pharmacokinetic properties of bispecifics in SCID mice

| | t_{1/2} | AUC_{0-∞} | CL | Vss |
|---|---|---|---|---|
| Molecules | (hr) | (mg*hr/mL) | (mL/h/kg) | (mL/kg) |
| V_{β}V_{α}-FTab-hb-1 | 24 | 0.75 | 22 | 350 |
| V_{β}V_{α}-FTab-hb-2 | 14 | 1.1 | 15 | 170 |
| V_{β}V_{α}-FTab-hb-5 | 6.4 | 0.77 | 21 | 140 |
| V_{β}V_{α}-FTab-KiH | 117.6 | 0.18 | 2.76 | 359 |

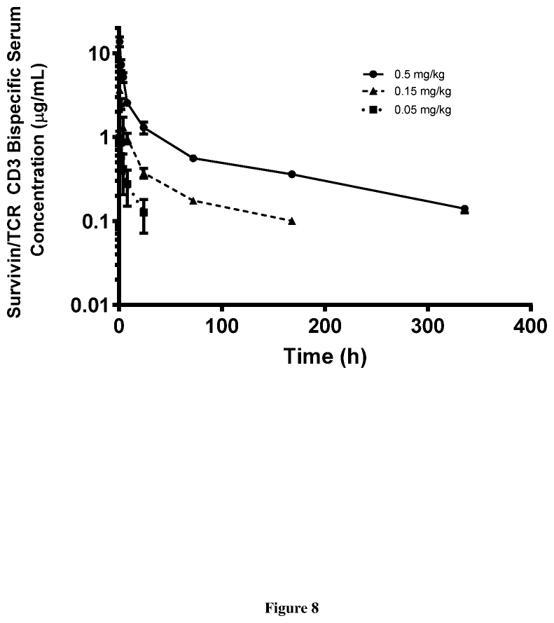

In addition, serum samples were analyzed for V_{β}V_{α}-FTab-KiH concentrations in a total anti-human MSD (Meso Scale Discovery) assay with electrochemiluminescent detection (FIG. 8). In the assay, total antibody was analyzed by employing a Bio anti-id capture reagent and a Sulfo anti-id mAb detection reagent. The linear range of the assay was 0.069-50 μg/mL, with a lower limit of quantitation (LLOQ) of 0.069 μg/mL. The serum concentrations at the first sampling time point (C_{0.5h}) were read directly from the concentration data for each monkey. Toxicokinetic parameters were calculated using Pharmacokinetics Laboratory Automation Software for Management and Analysis (PLASMA) Version 2.6.12 (SPaRCS, AbbVie) by non-compartmental analysis and the linear trapezoidal method.

### Example 8: Disulfide Bond Structure of Survivin TCR/CD3 Bispecific Molecules

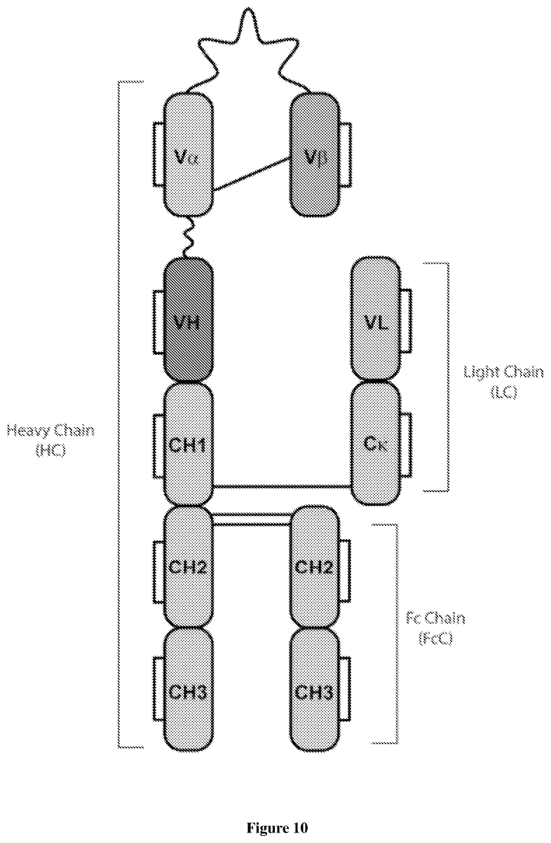

V_{β}V_{α}-FTab-KiH consists of one heavy chain subunit paired with one kappa light chain subunit and one Fc chain subunit, through disulfide bridges (FIG. 10). The heavy chain (i.e., Heavy Chain 1 of SEQ ID NO: 36) contains seven intrachain disulfide bridges between cysteines in positions 23 and 91, 43 and 235, 158 and 224, 288 and 362, 413 and 469, 530 and 590, and finally in positions 636 and 694. Among these intrachain disulfides, the disulfide bridge between cysteines 43 and 235 is an interdomain link connecting the V_{α} and V_{β} domain, whereas the rest are intradomain disulfide links. The light chain (i.e., Light Chain of SEQ ID NO: 76) contains two intrachain disulfide bridges; the first disulfide bridge is between cysteines in positions 23 and 87, and the second is between cysteines in positions 133 and 193. The Fc chain (i.e., Heavy Chain 2 of SEQ ID NO: 16) contains two intrachain disulfide bridges; the first disulfide bridge is between cysteines in positions 41 and 101, and the second is between cysteines in positions 147 and 205. In each molecule, the heavy chain is linked to the light chain by an interchain disulfide bridge between the cysteine in position 489 of the heavy chain and the cysteine in position 213 of the light chain. Each heavy chain is also paired with an Fc chain by two interchain disulfide bridges, one bridge between the cysteine in position 495 of the heavy chain and the cysteine in position 6 of the Fc chain, the other bridge between the cysteine in position 498 of the heavy chain and the cysteine in position 9 of the Fc chain.

### Example 9: Binding Specificity and Affinity Characterization of Survivin TCR/CD3 Bispecific Molecules

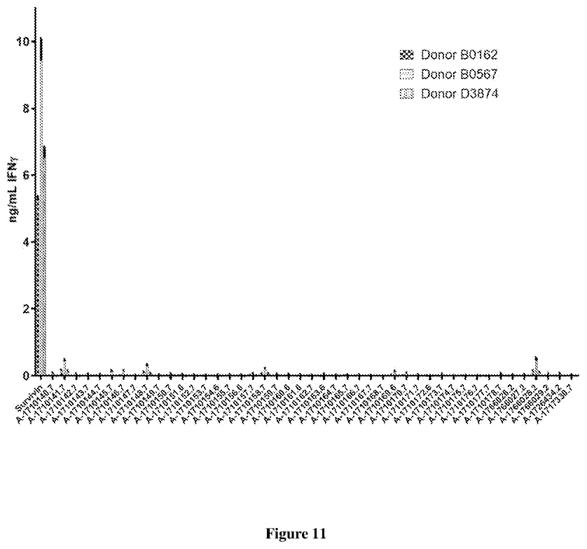

TCR specificity screen was carried out for V_{β}V_{α}-FTab-KiH (FIG. 11). T2 cells were seeded at 50,000 cells per well in a 96-well plate (Falcon #353077) in a volume of 50 μL AIM-V/5% hAB (Gibco #12055-091/Sigma #H4522) per well and the parental Survivin peptide, 43 homologous peptides, and 2 control peptides were added in 50 uL AIM-V/5% hAB to a final concentration of 20 μM with T2 and pre-incubated for 3-4 hours. 100,000 CD3+ cells were seeded in each well in a volume of 50 μL AIM-V/5% hAB per well. V_{β}V_{α}-FTab-KiH was diluted in AIM-V/5% hAB so that the final concentration in the co-culture was 1 nM in duplicate for each donor. Plates were incubated for 19 hours at 37° C. 5% CO₂. Supernatants were removed from each plate, transferred to a fresh 96-well plate and frozen at −80° C. until ready to assay for Interferon-γ secretion via ELISA. The TCR part of V_{β}V_{α}-FTab-KiH exhibited remarkable high specificity directed towards a Survivin-derived peptide (FIG. 11).

The Survivin/CD3 Bispecific Binding Kinetics to Survivin peptide/MHC for all test molecules are summarized in Table 3.

TABLE 3
Survivin/CD3 Bispecific Binding
Kinetics to Survivin peptide/MHC

| Bispecific | ka (1/Ms) | kd (1/s) | t½ (s) | K_{D} (M) |
|---|---|---|---|---|
| V_{β}V_{α}-FTab-KiH | 1.8E+05 | 2.5E−04 | 2758 | 1.4E−09 |
| V_{β}V_{α}-FTab-KiH-2 | 1.4E+05 | 2.8E−04 | 2506 | 2.0E−09 |

V_{β}V_{α}-FTab-KiH also has a high affinity to human CD3. The anti-CD3 part was described in Cole M S et al. (1999) Transplantation 68:563-571, the content of which is incorporated by reference herein in its entirety.

All publications, patents, patent applications and other documents cited in this application are hereby incorporated by reference in their entireties for all purposes to the same extent as if each individual publication, patent, patent application or other document were individually indicated to be incorporated by reference for all purposes.

While various specific embodiments have been illustrated and described, it will be appreciated that various changes can be made without departing from the spirit and scope of the invention(s).

We claim:

1. A bispecific molecule that binds to human Survivin and human CD3 comprising:
a) a sTCR comprising:
(1) a V_{β} comprising SEQ ID NO: 20 (CDR1), SEQ ID NO: 24 (CDR2), and SEQ ID NO: 28 (CDR3), and
(2) a V_{α} comprising SEQ ID NO: 19 (CDR1), SEQ ID NO: 23 (CDR2), and SEQ ID NO: 27 (CDR3),
wherein the V_{β} and V_{α} regions of the sTCR are connected via a first peptide linker (L1);
b) a Fab comprising:
(1) a heavy chain region comprising a V_{H} comprising SEQ ID NO: 21 (CDR1), SEQ ID NO: 25 (CDR2), and SEQ ID NO: 29 (CDR3); and a CH1 comprising the amino acid sequence of SEQ ID NO: 18, and
(2) a light chain region comprising a V_{L} comprising SEQ ID NO: 22 (CDR1), SEQ ID NO: 26 (CDR2), and SEQ ID NO: 30 (CDR3); and a C_{K} comprising the amino acid sequence of SEQ ID NO: 17,
wherein the V_{α} of the sTCR and the V_{H} of the Fab are connected via a second peptide linker (L2); and
c) a Fc region comprising:
(1) a first constant region comprising a first CH2 and a first CH3, wherein the first constant region (CH2CH3) comprises the amino acid sequence of SEQ ID NO: 13, and
(2) a second constant region comprising a second CH2′ and a second CH3′, wherein the second constant region (CH2′CH3′) comprises the amino acid sequence of SEQ ID NO: 16,
wherein the bispecific molecule is in the form of
V_{β}-L1-V_{α}-L2-V_{H}-CH1CH2CH3V_{L}-CκCH2′CH3′.


SEQUENCE LISTING (90 sequences)

SEQ ID NO: 1 - protein, 20 aa, Artificial Sequence
  synthetic peptide
GGGGSGGGGSGGGGSGGGGS

SEQ ID NO: 2 - protein, 115 aa, Artificial Sequence
  synthetic peptide
SQTIHQWPATLVQPVGSPLSLECTVEGTSNPNLYWYRQAAGRCLELLFYSVGIGQISSEVPQNLSASRPQDRQFILSSKKLLLSDSGFYLCAWSIGAEMFFGPGTRLTVLEDLKD

SEQ ID NO: 3 - protein, 115 aa, Artificial Sequence
  synthetic peptide
SQTIHQWPATLVQPVGSPLSLECTVEGTSNPNLYWYRQAAGRGPELLFYSVGIGQISSEVPQNLFASRPQDRQFILSSKKLLLSDSGFYLCAWSIGAEQFFGPGTRLTVLEDLKN

SEQ ID NO: 4 - protein, 115 aa, Artificial Sequence
  synthetic peptide
SQTIHQWPATLVQPVGSPLSLECTVEGTSNPNLYWYRQAAGRCLELLFYSVGIGQISSEVPQNLFASRPQDRQFILSSKKLLLSDSGFYLCAWSIGAEQFFGPGTRLTVLEDLKN

SEQ ID NO: 5 - protein, 115 aa, Artificial Sequence
  synthetic peptide
SQTIHQWPATLVQPVGSPLSLECTVEGTSNPNLYWYRQAAGRCLELLFYSVGIGQISSEVPQNLSASRPQDRQFILSSKKLLLSDSGFYLCAWSIGAEMFFGPGTRLTVLEDLKN

SEQ ID NO: 6 - protein, 111 aa, Artificial Sequence
  synthetic peptide
QKEVEQNSGPLSVPEGAIASLNCTYSDRYAQNFFWYRQYPGKSPELIMSIYSNGDKEDGRFTAQLNKASQYVSLLIRDSQPSDSATYLCAVSKGYKVFGCGTQLVVKPNIR

SEQ ID NO: 7 - protein, 111 aa, Artificial Sequence
  synthetic peptide
QKEVEQNSGPLSVPEGAIASLNCTYSDRYAQNFFWYRQYSGKSPELIMSIYSNGDKEDGRFTAQLNKASQYVSLLIRDSQPSDSATYLCAVSKGYKVFGDGTQLVVKPNIR

SEQ ID NO: 8 - protein, 111 aa, Artificial Sequence
  synthetic peptide
QKEVEQNSGPLSVPEGAIASLNCTYSDRYAQNFFWYRQYSGKSPELIMSIYSNGDKEDGRFTAQLNKASQYVSLLIRDSQPSDSATYLCAVSKGYKVFGCGTQLVVKPNIR

SEQ ID NO: 9 - protein, 120 aa, Artificial Sequence [sequence omitted]

SEQ ID NO: 10 - protein, 122 aa, Artificial Sequence
  synthetic peptide
EVQLVESGGGLVQPGGSLRLSCAASGYSFTGYTMNWVRQAPGKGLEWVALINPYKGVTTYADSVKGRFTISVDKSKNTAYLQMNSLRAEDTAVYYCARSGYYGDSDWYFDVWGQGTLVTVSS

SEQ ID NO: 11 - protein, 107 aa, Artificial Sequence
  synthetic peptide
DIQMTQSPSSLSASVGDRVTITCSASSSVSYMNWYQQKPGKAPKRLIYDTSKLASGVPSRFSGSGSGTDFTLTISSLQPEDFATYYCQQWSSNPPTFGGGTKVEIKR

SEQ ID NO: 12 - protein, 108 aa, Artificial Sequence
  synthetic peptide
DIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKR

SEQ ID NO: 13 - protein, 209 aa, Artificial Sequence
  synthetic peptide
SVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 14 - protein, 209 aa, Artificial Sequence
  synthetic peptide
CVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTAPVLDSDGSFRLRSDLTVDKSRWQQGNVFSCSVMHEALHNHYTQKSLSLSPGK

SEQ ID NO: 15 - protein, 209 aa, Artificial Sequence
  synthetic peptide
SVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDEMTKNQVSLTCLVKGFYPSDIAVEWESNGQPENNYKTTPPVLDSDGSFRLYSKLTVDKSRWQQGNVFSCSVMHEALHNHYTQKSLSLSPGK

SEQ ID NO: 16 - protein, 227 aa, Artificial Sequence
  synthetic peptide
DKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 17 - protein, 106 aa, Artificial Sequence
  synthetic peptide
TVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 18 - protein, 121 aa, Artificial Sequence
  synthetic peptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGP

SEQ ID NO: 19 - protein, 6 aa, Artificial Sequence
  synthetic peptide
DRYAQN

SEQ ID NO: 20 - protein, 6 aa, Artificial Sequence
  synthetic peptide
GTSNPN

SEQ ID NO: 21 - protein, 5 aa, Artificial Sequence
  synthetic peptide
SYTMH

SEQ ID NO: 22 - protein, 10 aa, Artificial Sequence
  synthetic peptide
SASSSVSYMN

SEQ ID NO: 23 - protein, 6 aa, Artificial Sequence
  synthetic peptide
IYSNGD

SEQ ID NO: 24 - protein, 5 aa, Artificial Sequence
  synthetic peptide
SVGIG

SEQ ID NO: 25 - protein, 17 aa, Artificial Sequence
  synthetic peptide
YINPRSGYTHYNQKLKD

SEQ ID NO: 26 - protein, 7 aa, Artificial Sequence
  synthetic peptide
DTSKLAS

SEQ ID NO: 27 - protein, 8 aa, Artificial Sequence
  synthetic peptide
AVSKGYKV

SEQ ID NO: 28 - protein, 9 aa, Artificial Sequence
  synthetic peptide
AWSIGAEMF

SEQ ID NO: 29 - protein, 11 aa, Artificial Sequence
  synthetic peptide
SAYYDYDGFAY

SEQ ID NO: 30 - protein, 9 aa, Artificial Sequence
  synthetic peptide
QQWSSNPPT

SEQ ID NO: 31 - protein, 9 aa, Artificial Sequence
  synthetic peptide
AWSIGAEQF

SEQ ID NO: 32 - protein, 10 aa, Artificial Sequence
  synthetic peptide
GYSFTGYTMN

SEQ ID NO: 33 - protein, 9 aa, Artificial Sequence
  synthetic peptide
LINPYKGVT

SEQ ID NO: 34 - protein, 13 aa, Artificial Sequence
  synthetic peptide
SGYYGDSDWYFDV

SEQ ID NO: 35 - protein, 121 aa, Artificial Sequence
  synthetic peptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSSDKTHTSPPCPAPELLGGP

SEQ ID NO: 36 - protein, 716 aa, Artificial Sequence
  synthetic peptide
SQTIHQWPATLVQPVGSPLSLECTVEGTSNPNLYWYRQAAGRCLELLFYSVGIGQISSEVPQNLSASRPQDRQFILSSKKLLLSDSGFYLCAWSIGAEMFFGPGTRLTVLEDLKDGGGGSGGGGSGGGGSGGGGSQKEVEQNSGPLSVPEGAIASLNCTYSDRYAQNFFWYRQYPGKSPELIMSIYSNGDKEDGRFTAQLNKASQYVSLLIRDSQPSDSATYLCAVSKGYKVFGCGTQLVVKPNIRGGGGSGGGGSGGGGSGGGGSQVQLVQSGAEVKKPGASVKVSCKASGYTFISYTMHWVRQAPGQGLEWMGYINPRSGYTHYNQKLKDKATLTADKSASTAYMELSSLRSEDTAVYYCARSAYYDYDGFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 37 - protein, 330 aa, Artificial Sequence
  synthetic peptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 38 - protein, 330 aa, Artificial Sequence
  synthetic peptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSSDKTHTSPPCPAPELLGGPCVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTAPVLDSDGSFRLRSDLTVDKSRWQQGNVFSCSVMHEALHNHYTQKSLSLSPGK

SEQ ID NO: 39 - protein, 330 aa, Artificial Sequence
  synthetic peptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSSDKTHTSPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDEMTKNQVSLTCLVKGFYPSDIAVEWESNGQPENNYKTTPPVLDSDGSFRLYSKLTVDKSRWQQGNVFSCSVMHEALHNHYTQKSLSLSPGK

SEQ ID NO: 40 - protein, 9 aa, Artificial Sequence
  synthetic peptide
LTLGEFLKL

SEQ ID NO: 41 - DNA, 60 bp, Artificial Sequence
  dna
ggaggaggaggatccggaggaggaggatctggcggcggcggcagcggcggcggcggctcc

SEQ ID NO: 42 - DNA, 345 bp, Artificial Sequence
  dna
tcacagactattcatcagtggcctgctactctggtgcagcctgtgggctctccactgagcctggagtgcaccgtggagggcacaagcaaccccaatctgtactggtataggcaggcagcaggcagatgtctggagctgctgttctacagcgtgggcatcggccagatcagctccgaggtgccacagaacctgtccgcatccaggccccaggacaggcagtttatcctgtctagcaagaagctgctgctgtccgattctggcttttacctgtgcgcatggagcatcggagcagagatgttctttggccctggcaccaggctgacagtgctggaggacctgaaggac

SEQ ID NO: 43 - DNA, 345 bp, Artificial Sequence
  dna
tcacagactattcatcagtggcctgctactctggtgcagcctgtgggctctccactgagcctggagtgcaccgtggagggcacaagcaaccccaatctgtactggtataggcaggcagcaggaagaggacctgagctgctgttctacagcgtgggcatcggccagatcagctccgaggtgccacagaacctgttcgcatccaggccccaggacaggcagtttatcctgtctagcaagaagctgctgctgtccgattctggcttttacctgtgcgcatggagcatcggagcagagcagttctttggcccaggcaccaggctgacagtgctggaggacctgaagaac

SEQ ID NO: 44 - DNA, 345 bp, Artificial Sequence
  dna
tcacagactattcatcagtggcctgctactctggtgcagcctgtgggctctccactgagcctggagtgcaccgtggagggcacaagcaaccccaatctgtactggtataggcaggcagcaggcagatgtctggagctgctgttctacagcgtgggcatcggccagatcagctccgaggtgccacagaacctgttcgcatccaggccccaggacaggcagtttatcctgtctagcaagaagctgctgctgtccgattctggcttttacctgtgcgcatggagcatcggagcagagcagttctttggccctggcaccaggctgacagtgctggaggacctgaagaac

SEQ ID NO: 45 - DNA, 345 bp, Artificial Sequence
  dna
tcacagactattcatcagtggcctgctactctggtgcagcctgtgggctctccactgagcctggagtgcaccgtggagggcacaagcaaccccaatctgtactggtataggcaggcagcaggcagatgtctggagctgctgttctacagcgtgggcatcggccagatcagctccgaggtgccacagaacctgtccgcatccaggccccaggacaggcagtttatcctgtctagcaagaagctgctgctgtccgattctggcttttacctgtgcgcatggagcatcggagcagagatgttctttggccctggcaccaggctgacagtgctggaggacctgaagaac

SEQ ID NO: 46 - DNA, 333 bp, Artificial Sequence
  dna
cagaaggaggtggagcagaatagcggaccactgtccgtgcctgagggagcaatcgcctctctgaactgtacctacagcgatagatatgcccagaatttcttttggtaccgccagtatcctggcaagtctcctgagctgatcatgagcatctactccaacggcgacaaggaggatggccggttcacagcccagctgaataaggcctctcagtatgtgagcctgctgatcagagactctcagcctagcgattccgccacctacctgtgcgccgtgagcaagggctataaggtgtttggctgtggcacacagctggtggtgaagccaaacatcagg

SEQ ID NO: 47 - DNA, 333 bp, Artificial Sequence
  dna
cagaaggaggtggagcagaatagcggaccactgtccgtgcctgagggagcaatcgcctctctgaactgtacctacagcgatagatatgcccagaatttcttttggtaccgccagtattccggcaagtctcccgagctgatcatgagcatctactccaacggcgacaaggaggatggccggttcacagcccagctgaataaggcctctcagtatgtgagcctgctgatcagagactctcagcccagcgattccgccacctacctgtgcgccgtgagcaagggctataaggtgtttggcgacggcacacagctggtggtgaagcctaacatcagg

SEQ ID NO: 48 - DNA, 333 bp, Artificial Sequence
  dna
cagaaggaggtggagcagaatagcggaccactgtccgtgcctgagggagcaatcgcctctctgaactgtacctacagcgatagatatgcccagaatttcttttggtaccgccagtattccggcaagtctcctgagctgatcatgagcatctactccaacggcgacaaggaggatggccggttcacagcccagctgaataaggcctctcagtatgtgagcctgctgatcagagactctcagcctagcgattccgccacctacctgtgcgccgtgagcaagggctataaggtgtttggctgtggcacacagctggtggtgaagccaaacatcagg

SEQ ID NO: 49 - DNA, 360 bp, Artificial Sequence
  dna
caggtgcagctggtgcagagcggagcagaggtgaagaagccaggagcctccgtgaaggtgtcttgcaaggccagcggctacaccttcatctcctatacaatgcactgggtgaggcaggcacctggacagggcctggagtggatgggctacatcaacccacgcagcggctacacccactataatcagaagctgaaggacaaggccaccctgacagccgataagtctgccagcaccgcctatatggagctgtcctctctgaggagcgaggacacagccgtgtactattgcgcccgctccgcctactatgactacgatggcttcgcctattggggccaaggaactctggtcaccgtttcttca

SEQ ID NO: 50 - DNA, 366 bp, Artificial Sequence
  dna
gaggtgcagctggtggagtccggcggcggcctggtgcagcccggcggctctctgaggctgagctgtgcagcatccggatactctttcaccggctatacaatgaactgggtgagacaggcaccaggcaagggcctggagtgggtggccctgatcaatccttacaagggcgtgaccacatatgccgacagcgtgaagggccgctttaccatctccgtggataagtctaagaacacagcctacctgcagatgaattccctgagggccgaggacaccgccgtgtactattgcgcacgcagcggatactatggcgactccgattggtatttcgacgtgtggggacagggcaccctggtgacagtgtcctct

SEQ ID NO: 51 - DNA, 321 bp, Artificial Sequence
  dna
gacatccagatgacccagagcccaagctccctgtccgcctctgtgggcgatagggtgaccatcacatgttctgcctcttcctccgtgagctacatgaactggtatcagcagaagcctggcaaggccccaaagcggctgatctacgataccagcaagctggcatccggagtgccaagccggttcagcggctccggctctggcaccgactttaccctgacaatctctagcctgcagcctgaggatttcgccacatactattgccagcagtggtcctctaatccccctacctttggcggcggcacaaaggtggagatcaaacga

SEQ ID NO: 52 - DNA, 324 bp, Artificial Sequence
  dna
gatatccagatgacccagtccccaagctccctgtctgccagcgtgggcgaccgggtgaccatcacatgtagagccagccaggatatcaggaactacctgaattggtatcagcagaagccaggcaaggcccccaagctgctgatctactatacatcccgcctggagtctggagtgccaagccggttttccggatctggaagcggaaccgactacaccctgacaatctctagcctgcagcctgaggatttcgccacatactattgccagcagggcaacaccctgccatggacatttggccagggcaccaaggtggagatcaagcgc

SEQ ID NO: 53 - DNA, 627 bp, Artificial Sequence
  dna
tcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacgccagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgtcctgcgctgtcaaaggcttctatcccagcgacatcgccgtggagtgggagagcaatgggcagccggagaacaactacaagaccacgcctcccgtgctggactccgacggctccttcttcctcgtgagcaagctcaccgtggacaagagcaggtggcagcaggggaacgtcttctcatgctccgtgatgcatgaggctctgcacaaccactacacgcagaagagcctctccctgtctccgggaaaa

SEQ ID NO: 54 - DNA, 627 bp, Artificial Sequence
  dna
tgcgtgttcctgtttccacccaagccaaaggacaccctgatgatctcccggaccccagaggtgacatgcgtggtggtggacgtgtctcacgaggatcccgaggtgaagttcaactggtacgtggatggcgtggaggtgcacaatgccaagaccaagcctcgggaggagcagtacaactccacctatagagtggtgtctgtgctgacagtgctgcaccaggactggctgaacggcaaggagtacaagtgcaaggtgagcaataaggccctgcctgccccaatcgagaagaccatctccaaggcaaagggacagccaagggagccacaggtgtatacactgcctccatccagagaggagatgaccaagaaccaggtgtctctgacatgtctggtgaagggcttttacccatctgatatcgccgtggagtgggagagcaatggccagcccgagaacaattataagaccacagcccctgtgctggactccgatggctctttccggctgagaagcgacctgaccgtggataagtcccggtggcagcagggcaacgtgttcagctgttccgtgatgcacgaagcactgcacaaccattacacccagaagtcactgtcactgtccccaggaaag

SEQ ID NO: 55 - DNA, 627 bp, Artificial Sequence
  dna
agcgtgttcctgtttccacccaagccaaaggacaccctgatgatctcccggaccccagaggtgacatgcgtggtggtggacgtgtctcacgaggatcctgaggtgaagttcaactggtacgtggatggcgtggaggtgcacaatgccaagaccaagccacgggaggagcagtacgcctccacctatagagtggtgtctgtgctgacagtgctgcaccaggactggctgaacggcaaggagtacaagtgcaaggtgagcaataaggccctgcctgccccaatcgagaagaccatctccaaggcaaagggacagccaagggagccacaggtgtatacactgcctccatccagagacgagatgaccaagaaccaggtgtctctgacatgtctggtgaagggcttttacccctctgatatcgccgtggagtgggagagcaatggccagcccgagaacaattataagaccacaccccctgtgctggactccgatggctctttccggctgtatagcaagctgaccgtggataagtcccggtggcagcagggcaacgtgttcagctgttccgtgatgcacgaggcactgcacaaccattacactcagaagtcactgtcactgtcaccaggaaaa

SEQ ID NO: 56 - DNA, 681 bp, Artificial Sequence
  dna
gacaaaactcacacatgcccaccgtgcccagcacctgaactgctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacgcaagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgcgaggagatgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggaaaa

SEQ ID NO: 57 - DNA, 318 bp, Artificial Sequence
  dna
accgtggcagcaccatccgtgttcatctttccaccctctgacgagcagctgaagagcggcacagcctccgtggtgtgcctgctgaacaatttctaccccagagaggccaaggtgcagtggaaggtggataacgccctgcagagcggcaattcccaggagtctgtgaccgagcaggacagcaaggattccacatattctctgagctccaccctgacactgtccaaggccgactacgagaagcacaaggtgtatgcctgcgaggtgacccaccagggcctgtctagccctgtgacaaagagctttaacagaggcgagtgt

SEQ ID NO: 58 - DNA, 363 bp, Artificial Sequence
  dna
gcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccg

SEQ ID NO: 59 - DNA, 18 bp, Artificial Sequence
  dna
gatagatatgcccagaat

SEQ ID NO: 60 - DNA, 18 bp, Artificial Sequence
  dna
ggcacaagcaaccccaat

SEQ ID NO: 61 - DNA, 15 bp, Artificial Sequence
  dna
tcctatacaatgcac

SEQ ID NO: 62 - DNA, 30 bp, Artificial Sequence
  dna
tctgcctcttcctccgtgagctacatgaac

SEQ ID NO: 63 - DNA, 18 bp, Artificial Sequence
  dna
atctactccaacggcgac

SEQ ID NO: 64 - DNA, 15 bp, Artificial Sequence
  dna
agcgtgggcatcggc

SEQ ID NO: 65 - DNA, 51 bp, Artificial Sequence
  dna
tacatcaacccacgcagcggctacacccactataatcagaagctgaaggac

SEQ ID NO: 66 - DNA, 21 bp, Artificial Sequence
  dna
gataccagcaagctggcatcc

SEQ ID NO: 67 - DNA, 24 bp, Artificial Sequence
  dna
gccgtgagcaagggctataaggtg

SEQ ID NO: 68 - DNA, 27 bp, Artificial Sequence
  dna
gcatggagcatcggagcagagatgttc

SEQ ID NO: 69 - DNA, 33 bp, Artificial Sequence
  dna
tccgcctactatgactacgatggcttcgcctat

SEQ ID NO: 70 - DNA, 27 bp, Artificial Sequence
  dna
cagcagtggtcctctaatccccctacc

SEQ ID NO: 71 - DNA, 27 bp, Artificial Sequence
  dna
gcatggagcatcggagcagagcagttc

SEQ ID NO: 72 - DNA, 30 bp, Artificial Sequence
  dna
ggatactctttcaccggctatacaatgaac

SEQ ID NO: 73 - DNA, 27 bp, Artificial Sequence
  dna
ctgatcaatccttacaagggcgtgacc

SEQ ID NO: 74 - DNA, 39 bp, Artificial Sequence
  dna
agcggatactatggcgactccgattggtatttcgacgtg

SEQ ID NO: 75 - DNA, 363 bp, Artificial Sequence
  dna
gccagcaccaagggcccttccgtgtttcccctggccccttctagcaagtccacctctggaggaacagccgccctgggctgtctggtgaaggattacttccccgagcctgtgacagtgtcttggaacagcggcgccctgacctctggagtgcacacatttcctgccgtgctgcagtcctctggcctgtactccctgagctccgtggtgaccgtgccatctagctccctgggcacccagacatatatctgcaacgtgaatcacaagccaagcaatacaaaggtggacaagaaggtggagcccaagtctagcgataagacccacacatccccaccttgcccagcaccagagctgctgggcggccct

SEQ ID NO: 76 - protein, 213 aa, Artificial Sequence
  synthetic peptide
DIQMTQSPSSLSASVGDRVTITCSASSSVSYMNWYQQKPGKAPKRLIYDTSKLASGVPSRFSGSGSGTDFTLTISSLQPEDFATYYCQQWSSNPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 77 - DNA, 990 bp, Artificial Sequence
  dna
gcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacgccagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgtcctgcgctgtcaaaggcttctatcccagcgacatcgccgtggagtgggagagcaatgggcagccggagaacaactacaagaccacgcctcccgtgctggactccgacggctccttcttcctcgtgagcaagctcaccgtggacaagagcaggtggcagcaggggaacgtcttctcatgctccgtgatgcatgaggctctgcacaaccactacacgcagaagagcctctccctgtctccgggaaaa

SEQ ID NO: 78 - DNA, 990 bp, Artificial Sequence
  dna
gccagcaccaagggcccttccgtgtttcccctggccccttctagcaagtccacctctggaggaacagccgccctgggctgtctggtgaaggattacttccccgagcctgtgacagtgtcttggaacagcggcgccctgacctctggagtgcacacatttcctgccgtgctgcagtcctctggcctgtactccctgagctccgtggtgaccgtgccatctagctccctgggcacccagacatatatctgcaacgtgaatcacaagccaagcaatacaaaggtggacaagaaggtggagcccaagtctagcgataagacccacacatccccaccttgcccagcaccagagctgctgggcggcccttgcgtgttcctgtttccacccaagccaaaggacaccctgatgatctcccggaccccagaggtgacatgcgtggtggtggacgtgtctcacgaggatcccgaggtgaagttcaactggtacgtggatggcgtggaggtgcacaatgccaagaccaagcctcgggaggagcagtacaactccacctatagagtggtgtctgtgctgacagtgctgcaccaggactggctgaacggcaaggagtacaagtgcaaggtgagcaataaggccctgcctgccccaatcgagaagaccatctccaaggcaaagggacagccaagggagccacaggtgtatacactgcctccatccagagaggagatgaccaagaaccaggtgtctctgacatgtctggtgaagggcttttacccatctgatatcgccgtggagtgggagagcaatggccagcccgagaacaattataagaccacagcccctgtgctggactccgatggctctttccggctgagaagcgacctgaccgtggataagtcccggtggcagcagggcaacgtgttcagctgttccgtgatgcacgaagcactgcacaaccattacacccagaagtcactgtcactgtccccaggaaag

SEQ ID NO: 79 - DNA, 990 bp, Artificial Sequence
  dna
gccagcaccaagggaccatccgtgtttccactggcaccttctagcaagtccacctctggaggaacagccgccctgggctgtctggtgaaggattacttccccgagcctgtgacagtgtcttggaacagcggcgccctgacctctggagtgcacacatttccagccgtgctgcagtcctctggcctgtactccctgagctccgtggtgaccgtgccctctagctccctgggcacccagacatatatctgcaacgtgaatcacaagccaagcaatacaaaggtggacaagaaggtggagcccaagtctagcgataagacccacacatccccaccttgcccagcaccagagctgctgggcggccctagcgtgttcctgtttccacccaagccaaaggacaccctgatgatctcccggaccccagaggtgacatgcgtggtggtggacgtgtctcacgaggatcctgaggtgaagttcaactggtacgtggatggcgtggaggtgcacaatgccaagaccaagccacgggaggagcagtacgcctccacctatagagtggtgtctgtgctgacagtgctgcaccaggactggctgaacggcaaggagtacaagtgcaaggtgagcaataaggccctgcctgccccaatcgagaagaccatctccaaggcaaagggacagccaagggagccacaggtgtatacactgcctccatccagagacgagatgaccaagaaccaggtgtctctgacatgtctggtgaagggcttttacccctctgatatcgccgtggagtgggagagcaatggccagcccgagaacaattataagaccacaccccctgtgctggactccgatggctctttccggctgtatagcaagctgaccgtggataagtcccggtggcagcagggcaacgtgttcagctgttccgtgatgcacgaggcactgcacaaccattacactcagaagtcactgtcactgtcaccaggaaaa

SEQ ID NO: 80 - DNA, 2148 bp, Artificial Sequence
  dna
tcacagactattcatcagtggcctgctactctggtgcagcctgtgggctctccactgagcctggagtgcaccgtggagggcacaagcaaccccaatctgtactggtataggcaggcagcaggcagatgtctggagctgctgttctacagcgtgggcatcggccagatcagctccgaggtgccacagaacctgtccgcatccaggccccaggacaggcagtttatcctgtctagcaagaagctgctgctgtccgattctggcttttacctgtgcgcatggagcatcggagcagagatgttctttggccctggcaccaggctgacagtgctggaggacctgaaggacggcggcggaggatctgggggaggcggaagcggaggcggaggatccggcggagggggatctcagaaggaggtggagcagaatagcggaccactgtccgtgcctgagggagcaatcgcctctctgaactgtacctacagcgatagatatgcccagaatttcttttggtaccgccagtatcctggcaagtctcctgagctgatcatgagcatctactccaacggcgacaaggaggatggccggttcacagcccagctgaataaggcctctcagtatgtgagcctgctgatcagagactctcagcctagcgattccgccacctacctgtgcgccgtgagcaagggctataaggtgtttggctgtggcacacagctggtggtgaagccaaacatcaggggcggcggaggatctgggggaggcggaagcggaggcggaggatccggcggagggggatctcaggtgcagctggtgcagagcggagcagaggtgaagaagccaggagcctccgtgaaggtgtcttgcaaggccagcggctacaccttcatctcctatacaatgcactgggtgaggcaggcacctggacagggcctggagtggatgggctacatcaacccacgcagcggctacacccactataatcagaagctgaaggacaaggccaccctgacagccgataagtctgccagcaccgcctatatggagctgtcctctctgaggagcgaggacacagccgtgtactattgcgcccgctccgcctactatgactacgatggcttcgcctattggggccaaggaactctggtcaccgtttcttcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacgccagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgtcctgcgctgtcaaaggcttctatcccagcgacatcgccgtggagtgggagagcaatgggcagccggagaacaactacaagaccacgcctcccgtgctggactccgacggctccttcttcctcgtgagcaagctcaccgtggacaagagcaggtggcagcaggggaacgtcttctcatgctccgtgatgcatgaggctctgcacaaccactacacgcagaagagcctctccctgtctccgggaaaa

SEQ ID NO: 81 - protein, 11 aa, Artificial Sequence
  synthetic peptide
RASQDIRNYLN

SEQ ID NO: 82 - protein, 7 aa, Artificial Sequence
  synthetic peptide
YTSRLES

SEQ ID NO: 83 - protein, 9 aa, Artificial Sequence
  synthetic peptide
QQGNTLPWT

SEQ ID NO: 84 - DNA, 33 bp, Artificial Sequence
  dna
agagccagccaggatatcaggaactacctgaat

SEQ ID NO: 85 - DNA, 21 bp, Artificial Sequence
  dna
tatacatcccgcctggagtct

SEQ ID NO: 86 - DNA, 27 bp, Artificial Sequence
  dna
cagcagggcaacaccctgccatggaca

SEQ ID NO: 87 - DNA, 639 bp, Artificial Sequence
  dna
gacatccagatgacccagagcccaagctccctgtccgcctctgtgggcgatagggtgaccatcacatgttctgcctcttcctccgtgagctacatgaactggtatcagcagaagcctggcaaggccccaaagcggctgatctacgataccagcaagctggcatccggagtgccaagccggttcagcggctccggctctggcaccgactttaccctgacaatctctagcctgcagcctgaggatttcgccacatactattgccagcagtggtcctctaatccccctacctttggcggcggcacaaaggtggagatcaaacgaaccgtggcagcaccatccgtgttcatctttccaccctctgacgagcagctgaagagcggcacagcctccgtggtgtgcctgctgaacaatttctaccccagagaggccaaggtgcagtggaaggtggataacgccctgcagagcggcaattcccaggagtctgtgaccgagcaggacagcaaggattccacatattctctgagctccaccctgacactgtccaaggccgactacgagaagcacaaggtgtatgcctgcgaggtgacccaccagggcctgtctagccctgtgacaaagagctttaacagaggcgagtgt

SEQ ID NO: 88 - protein, 716 aa, Artificial Sequence
  synthetic peptide
SQTIHQWPATLVQPVGSPLSLECTVEGTSNPNLYWYRQAAGRCLELLFYSVGIGQISSEVPQNLSASRPQDRQFILSSKKLLLSDSGFYLCAWSIGAEMFFGPGTRLTVLEDLKNGGGGSGGGGSGGGGSGGGGSQKEVEQNSGPLSVPEGAIASLNCTYSDRYAQNFFWYRQYPGKSPELIMSIYSNGDKEDGRFTAQLNKASQYVSLLIRDSQPSDSATYLCAVSKGYKVFGCGTQLVVKPNIRGGGGSGGGGSGGGGSGGGGSQVQLVQSGAEVKKPGASVKVSCKASGYTFISYTMHWVRQAPGQGLEWMGYINPRSGYTHYNQKLKDKATLTADKSASTAYMELSSLRSEDTAVYYCARSAYYDYDGFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 89 - DNA, 2148 bp, Artificial Sequence
  dna
tcacagactattcatcagtggcctgctactctggtgcagcctgtgggctctccactgagcctggagtgcaccgtggagggcacaagcaaccccaatctgtactggtataggcaggcagcaggcagatgtctggagctgctgttctacagcgtgggcatcggccagatcagctccgaggtgccacagaacctgtccgcatccaggccccaggacaggcagtttatcctgtctagcaagaagctgctgctgtccgattctggcttttacctgtgcgcatggagcatcggagcagagatgttctttggccctggcaccaggctgacagtgctggaggacctgaagaacggaggaggaggatccggaggaggaggatctggcggcggcggcagcggcggcggcggctcccagaaggaggtggagcagaatagcggaccactgtccgtgcctgagggagcaatcgcctctctgaactgtacctacagcgatagatatgcccagaatttcttttggtaccgccagtatcctggcaagtctcctgagctgatcatgagcatctactccaacggcgacaaggaggatggccggttcacagcccagctgaataaggcctctcagtatgtgagcctgctgatcagagactctcagcctagcgattccgccacctacctgtgcgccgtgagcaagggctataaggtgtttggctgtggcacacagctggtggtgaagccaaacatcaggggaggaggaggatccggaggaggaggatctggcggcggcggcagcggcggcggcggctcccaggtgcagctggtgcagagcggagcagaggtgaagaagccaggagcctccgtgaaggtgtcttgcaaggccagcggctacaccttcatctcctatacaatgcactgggtgaggcaggcacctggacagggcctggagtggatgggctacatcaacccacgcagcggctacacccactataatcagaagctgaaggacaaggccaccctgacagccgataagtctgccagcaccgcctatatggagctgtcctctctgaggagcgaggacacagccgtgtactattgcgcccgctccgcctactatgactacgatggcttcgcctattggggccaaggaactctggtcaccgtttcttcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacgccagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgtcctgcgctgtcaaaggcttctatcccagcgacatcgccgtggagtgggagagcaatgggcagccggagaacaactacaagaccacgcctcccgtgctggactccgacggctccttcttcctcgtgagcaagctcaccgtggacaagagcaggtggcagcaggggaacgtcttctcatgctccgtgatgcatgaggctctgcacaaccactacacgcagaagagcctctccctgtctccgggaaaa

SEQ ID NO: 90 - protein, 6 aa, Artificial Sequence
  synthetic peptide
HHHHHH